(19) United States
(12) Patent Application Publication
(10) Pub. No.: US 2005/0234030 A1
(43) Pub. Date: Oct. 20, 2005

(54) Modulators of CRTH2, COX-2 and FAAH

(76) Inventors: Wilmin Bartolini, Amesbury, MA (US); Brian M. Cali, Arlington, MA (US); Barbara Chen, Watertown, MA (US); Yueh-Tyng Chien, Newton, MA (US); Mark G. Currie, Sterling, MA (US); G. Todd Milne, Brookline, MA (US); James Philip Pearson, Cambridge, MA (US); John Jeffrey Talley, Somerville, MA (US); Jing Jing Yang, Boxborough, MA (US); Craig Zimmerman, Topsfield, MA (US)

Correspondence Address: FISH & RICHARDSON PC, P.O. BOX 1022, MINNEAPOLIS, MN, 55440-1022, US

(21) Appl. No.: 11/028,896

(22) Filed: Jan. 3, 2005

Related U.S. Application Data
(60) Provisional application No. 60/563,589, filed on Apr. 20, 2004.
(60) Provisional application No. 60/570,620, filed on May 13, 2004.
(60) Provisional application No. 60/585,102, filed on Jul. 1, 2004.

(51) Int. Cl. A61K 31/53; A61K 31/501; A61K 31/497; A61K 31/541; A61K 31/5377; A61K 31/496; C07D 43/02; C07D 417/02; C07D 413/02

(52) U.S. Cl. 514/183; 514/241; 514/252.06; 514/255.05; 544/238; 544/209; 544/405; 548/495; 514/419; 514/227.5; 514/232.2; 514/254.05; 544/60; 544/373; 544/143; 514/406; 514/365; 514/374; 514/414

(57) ABSTRACT

Certain substituted indoles that are modulators of one or more or of CRTH2, COX-2 AND FAAH are described. The compounds are useful for treatment of pain and/or inflammation as well as other disorders.

Under 35 USC §119(e)(1), this application claims the benefit of prior U.S. Provisional Application Ser. Nos. 60/563,589, filed Apr. 20, 2004; 60/570,620, filed May 13, 2004; and 60/585,102, filed Jul. 1, 2004, the entire contents of which are hereby incorporated by reference. Under 35 U.S.C. §120, this application claims the benefit of prior U.S. application Ser. No. 10/883,900, filed Jul. 12, 2004; Ser. No. 10/859,335, filed Jun. 1, 2004; Ser. No. 10/951,542, filed Sep. 27, 2004; and Ser. No. 10/979,794, filed Nov. 1, 2004, the entire contents of which are hereby incorporated by reference.

### BACKGROUND

Cox Inhibitors

Cyclooxygenases play an essential role in prostaglandin synthesis. Cyclooxygenase-1 (COX-1) is constitutive and relatively long-lived, whereas cyclooxygenase-2 (COX-2) is inducible and relatively short-lived. COX-1 is thought to be responsible for maintaining basal level prostaglandin production, which is important for normal gastrointestinal and renal function. COX-2 is induced by certain inflammatory agents, hormones, growth factors, cytokines, and other agents. COX-2 plays a significant role in prostaglandin synthesis within inflammatory cells such as macrophages and monocytes, and prostaglandin production associated with COX-2 induction can have a deleterious effect on the body. Thus, to reduce unwanted inflammation and to treat certain other conditions, it can be desirable to inhibit COX-2 activity without significantly inhibiting COX-1 activity.

Many non-steroidal anti-inflammatory drugs (NSAIDs) inhibit both COX-1 and COX-2. These non-selective inhibitors include indomethacin (Shen et al. 1963 J Am Chem Soc 85:4881; 4-chlorobenzoyl-5-methoxy-2-methyl-1H-indole-3-acetic acid). It is desirable to identify NSAIDs that inhibit COX-2 activity, but do not significantly inhibit COX-1 activity at physiological levels where COX-2 activity is significantly inhibited. Such selective inhibitors are expected to have the desirable anti-inflammatory, anti-pyretic, and analgesic properties associated with NSAIDs, while having reduced or no gastrointestinal or renal toxicity.

Subsequent to indomethacin administration, the unchanged parent compound, the desmethyl metabolite (O-desmethylindomethacin), the desbenzoyl metabolite (N-deschlorobenzoylindomethacin) and the desmethy-desbenzoyl metabolite (O-desmethy-N-deschlorobenzoylindomethacin) can be found in plasma in significant amounts (Strachman et al. 1964 J Am Chem Soc 8:799; Helleberg 1981 Clin Pharmacokinet 6:245), all in an unconjugated form (Harman et al. 1964 J Pharmocol Exp Therap 143:215). It has been reported that all three metabolites are devoid of anti-inflammatory activity (Helleberg 1981 Clin Pharmacokine. 6:245 and Duggan et al. 1972 Pharmacol and Exp Ther 181:562), although it has also been reported that the desmethyl metabolite has some ability to inhibit prostaglandin synthesis (Shen et al. 1977 Adv Drug Res 12:90).

Indomethacin derivatives in which the benzoyl group has been replaced by a 4-bromobenzyl group or the acetic acid side chain has been extended exhibit greater selectivity for inhibition of COX-2 relative to COX-1 (Black et al. 1996 Bioorganic & Medicinal Chem Lett 6:725 and Black et al. 1997 Advances in Experimental Medicine and Biology 407:73). In addition, synthesis methodology has been demonstrated for the preparation of indomethacin analogues, some of which do not inhibit cyclooxygenases (Touhey et al. 2002 Eur J Cancer 38:1661).

FAAH Inhibitors

Many fatty acid amides are known to have analgesic activity. A number of fatty acid amides (e.g., arachidonyl amino acids and anandamide) induce analgesia in animal models of pain (see, for example, Walker et al. 1999 Proc Natl Acad Sci 96:12198, Fride and Mechoulam 1993 Eur J Pharmacol 231:313). Anandamide and certain other fatty acid amides (e.g., N-palmitoyl ethanolamine, N-oleoyl ethanolamide, oleamide, 2-arachidonoylglycerol) are cleaved and inactivated by fatty acid amide hydrolase (FAAH) (Deutsch et al. 2003 Prostaglandins Leukot Essent Fatty Acids 66:201; and Cravatt and Lichtman 2003 Current Opinion in Chemical Biology 7:469).

Inhibition of FAAH is expected to lead to an increase in the level of anandamide and other fatty acid amides. This increase in fatty acid amides may lead to an increase in the nociceptive threshold. Thus, inhibitors of FAAH are useful in the treatment of pain. Such inhibitors might also be useful in the treatment of other disorders that can be treated using fatty acid amides or modulators of cannabinoid receptors (e.g., anxiety, eating disorders, and cardiovascular disorders). NPAA (N-palmitoylethanolamine acid anhydrolase) is a hydrolase that breaks down N-palmitoyl ethanolamine (PEA), a fatty acid amide. PEA is a naturally occurring substrate for the cannabinoid receptor 2 (CB2 receptor). Inhibition of NPAA may lead to increased PEA levels. Accordingly, NPAA inhibitors may be useful in the treatment of inflammation and nociceptive pain control.

In addition, there is evidence (see, e.g., Weber et al. 2004 J. Lipid Res. 45:757) that when FAAH activity is reduced or absent, one of its substrates, anandamide, acts as a substrate for COX-2 that can be converted to a prostamide. Thus, certain prostamides may be elevated in the presence of an FAAH inhibitor. Given that certain prostamides are associated with reduced intraocular pressure and ocular hypotensivity, FAAH inhibitors may be useful agents for treating glaucoma.

CRTH2 Modulators

CRTH2 is a G_{αi} protein-coupled receptor that is thought to be involved in both mediating PGD₂-induced chemoattraction and in activation of specific cell types involved in allergic inflammation. It has been reported that CRTH2 is expressed by Th2 cells, eosinophils and basophils, but not by Th1 cells, B cells or NK cells. (Nagata et al. 1999 FEBS Letters 459:195-199).

PGD₂ is produced by allergen-activated mast cells and has been implicated in various allergic diseases as a pro-inflammatory mediator, although it may have anti-inflammatory activity in certain situations (Ajuebor et al. 2000 Am J Physiol Gastrointest Liver Physiol 279:G238-44). CRTH2 receptor is a high affinity receptor for PGD₂ as is DP, a G_{αs} protein-coupled receptor.

CRTH2 agonists activate eosinophils, basophils and Th2 cells in vitro, resulting in induction of actin polymerization, calcium influx, CD11b expression and chemotaxis (Monneret et al 2003 J Pharmacol Exp Ther 304:349-55). An in vivo study has demonstrated that injection of a CRTH2 agonist can elicit transient recruitment of eosinophils from bone marrow into the blood (Shichijo 2003 J Pharmacol Exp Ther 307:518-525). A genetic study of African American and Chinese cohorts found that polymorphisms in CRTH2 were tightly associated with asthma susceptibility (Huang et al. 2004 Hum Mol. Genet 2791). It has been suggested that modulators of CRTH2 may be useful in the prevention and/or treatment of allergic asthma and other allergic disorders (US 2002/0022218 A1 and WO 03/066047). Recruitment and/or activation of eosinophils, basophils and Th2 cells is a prominent feature of the changes that occur in the asthmatic lung. Similar activation of these cell types, or subsets thereof, are believed to play an important role in the etiology of other diseases, including eosinophilic esophagitis and atopic dermatitis (Arora and Yamakazi 2004 Clin Gastroenterol Hepatol 2:523-30; Kiehl et al. 2001 Br J Dermatol 145:720-729). This fact, combined with the fact that CRTH2 mediates PGD₂-induced chemotaxis, suggests that compounds that alter chemotaxis by modulating CRTH2 activity could be useful in controlling chronic airway inflammation, allergic rhinitis, atopic dermatitis, chronic obstructive pulmonary disease (COPD), or eosinophilic esophagitis. Thus, CRTH2 antagonists that reduce the ability of Th2 cells and eosinophils to respond to mast-cell derived PGD₂ could be useful for preventing and/or treating allergic disorders such as allergic rhinitis and asthma.

It is often found that agonists induce desensitization of the cell system by promoting internalization and down regulation of the cell surface receptor (Int Immunol 15:29-38, 2003). Therefore, certain CRTH2 agonists may be therapeutically useful because they can cause the desensitization of PGD₂-responsive cells. It has been shown that certain CRTH2 agonists can induce desensitization of PGD₂-responsive cells to subsequent activation by a CRTH2 agonist (see, e.g., Yoshimura-Uchiyama et al. 2004 Clin Exp Allergy 34:1283-1290). Importantly, CRTH2 agonists may also cause cross-desensitization. Cross-desensitization, which can occur in many cell-signaling systems, refers to a phenomena whereby an agonist for one receptor can reduce or eliminate sensitivity of a cell type to an unrelated agonist/receptor signaling system. For example, it is known that treatment with the CRTH2 agonist indomethacin reduces expression of CCR3, the receptor for the chemoattractant, eotaxin (Stubbs et al. 2002, J Biol Chem 277:26012-26020).

### SUMMARY

The invention features compounds having Formula I or Formula II, pharmaceutically acceptable salts thereof, pharmaceutical compositions comprising such compounds and methods for treating a patient by administering such pharmaceutical compositions alone or in combination with one or more other therapeutic agents. Thus, the invention features compounds having either Formula I or Formula II:

The invention features a compound having the formula: 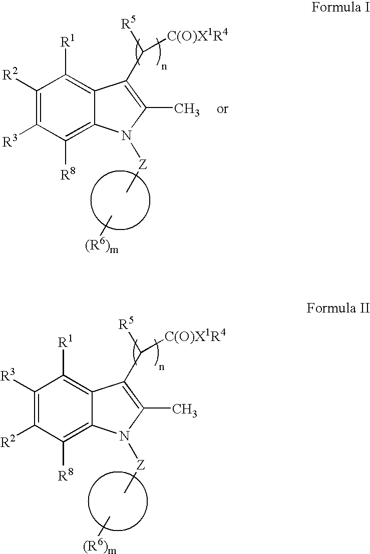
wherein:
R¹ is: H or a halogen;
R² is: H, a halogen, or R²⁸O— wherein
R^{2B} is selected from:
(a) H;
(b) C₁ to C₆ alkyl or a C₂ to C₆ alkenyl that is optionally independently substituted with one or more halogen; —OH, —NH₂, —C(O)OH; 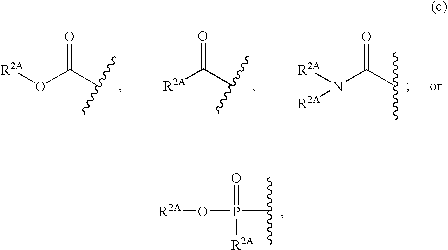
   wherein each R^{2A} is independently: H, a C₁ to C₆ alkyl, a C₂ to C₆ alkenyl, a C₂ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂;
R³ is H or a halogen;
X¹ is —O—, —S—, —N(H)— or —N(H)S(O₂)—;
Z is 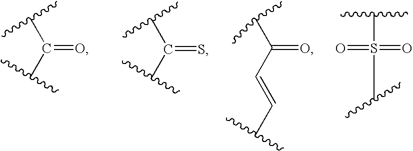
   or C;
R⁴ is H; a C₁ to C₁₀ alkyl; a C₂-C₁₀ alkenyl; a C₂-C₁₀ alkynyl; a C₃ to C₈ cycloalkyl; a C₁ to C₆ hydroxyalkyl; a hydroxyl substituted C₆ to C₈ aryl; a primary, secondary or tertiary, C₁ to C₆ alkylamino; primary, secondary or tertiary C₆ to C₈ arylamino; C₂ to C₆ alkylcarboxylic acid; a C₁ to C₆ alkylester; a C₆ to C₈ aryl; a C₆ to C₈ arylcarboxylic acid; a C₆ to C₈ arylester; a C₆ to C₈ aryl substituted C, to C₆ alkyl; a 4 to 8 membered heterocyclic alkyl or heteroaryl wherein the heteroatoms are selected from O, S, S(O)₂, N, and S(O); an alkyl-substituted or aryl-substituted a 4 to 8 membered heterocyclic alkyl or heteroaryl wherein the heteroatoms are selected from O, S, S(O)₂, N, and S(O), wherein one or more H within R⁴ can be substituted by a halogen, —OH, or —C(O)OH, —NH₂;
n is 1,2,3,4 or 5;

Each R⁵ is independently: H, an optionally substituted C₁-C₄ alkyl, wherein the substituents are independently selected from a halogen and —OH; 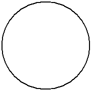
represents a C₃-C₆ saturated carbocycle, a C₆ aryl, C₃-C₆ non-saturated, non-aromatic carbocycle, a 6-membered heteroaryl having 1, 2, 3, 4 or 5 heteroatoms independently selected from O, S, S(O)₂, N, S(O) and N(R⁷) or a 3- to 7-membered saturated or non-saturated heterocycle having 1, 2, 3, 4 or 5 heteroatoms independently selected from O, S, S(O)₂, N, S(O) and N(R⁷);
each R⁶ is independently H, a halogen, —CH₃, —CN, —OCH₃, —SCH₃, —SCF₃, —OCH₂CF₃ or —CH₂CH₃ wherein one or more H can be replaced by a halogen;
m=1, 2, 3, 4, or 5;
R⁷ is: H, a halogen, —CH₃, —CN, —OCH₃, —SCH₃, or —CH₂CH₃ wherein one or more H can be replaced by a halogen; and
R⁸ is: H, a halogen or —CH₃, wherein one or more H can be replaced by a halogen.

In certain embodiments, each R⁶ is independently a halogen, —CH₃, —CN, —OCH₃, —SCH₃, —SCF₃, —OCH₂CF₃ or —CH₂CH₃ wherein one or more H can be replaced by a halogen

In certain embodiments, 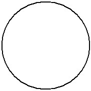
represents a C₃-C₆ saturated carbocycle (e.g., cyclohexyl, cyclopentyl, cyclobutyl or cyclopropyl). In certain embodiments 4 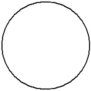
represents a C₃-C₆ non-saturated, non-aromatic carbocycle (e.g., cyclohexenyl, a cyclopentenyl, or cyclobutenyl). In certain embodiments 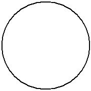
represents a 6-membered heteroaryl (e.g., pyrazine, pyridazine, triazine, tetrazine, or pentazine). In certain embodiments 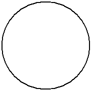
represents a 3- to 7-membered saturated heterocycle (e.g., piperidine, piperazine, morpholine, thiomorpholine, thiomorpholine sulfoxide, thiomorpholine sulfone, tetrahydropyran, tetrahydrothiopyran, or dioxane). In certain embodiments 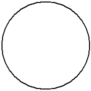
represents a 3- to 7-membered non-saturated heterocycle (e.g., thiphene, furan, pyrrole, thaizole, oxazole, imidizole, isothazole, isoxazole pyrazole, triazole, tetrazole, oxadiazole, oxatriazole or thiadiazole)

In various embodiments: R¹ is H; R¹ is a halogen (e.g., F or Cl); R² is R^{2B}O— and R^{2B} is a substituted C₁ to C₆ alkyl or a substituted C₂ to C₆ alkenyl; R² is R^{2B}O— and R^{2B} is not substituted; R² is R^{2B}O— and R^{2B} is a C₁ to C₆ alkyl or a C₂ to C₆ alkenyl optionally substituted with one or more halogen; R² is R^{2B}O— and R^{2B} is a C₁ to C₃ alkyl or alkenyl; R² is R^{2B}O— and R^{2B} is a C₁ to C₃ alkyl; R² is R^{2B}O— and R^{2B} is a methyl group or an ethyl group; R² is substituted only with a halogen; R² is H; R³ is a halogen; R³ is Cl; R³ is F; X¹ is —O—; X¹ is —S—; X¹ is —N(H)—; X¹ is —N(H)S(O)₂—; R⁶ is selected from: —CH₃, —CF₂H, —CH₂F, —CF₃, —CN, —OCF₂H, —OCH₃, —SCF₃, —SCF₂H, —SCH₃, —CH₂CH₃ and —OCF₃; selected from: —CH₃, —CF₂H, —CH₂F, —CF₃, —CN, —OCF₂H, —OCH₃, —SCF₃, —SCF₂H, —SCH₃, —CH₂CH₃ and —OCF₃; n is 1 or 2; m is 1 or 2; m is 1 or 2 and other than H R⁵ is a methyl group or an ethyl group; X¹ is 0 and R⁴ is H; XI is 0 and R⁴ is other than H; R⁴ is an optionally independently substituted C₃ to C₁₀ branched alkyl; R⁴ is a C₁ to C₁₀ alkyl; R⁴ is a C₄ to C₈ cycloalkyl; R⁴ is a C₁ to C₆ hydroxy substituted alkyl; R⁴ is a hydroxyl substituted C₄ to C₈ aryl; R⁴ is a primary, secondary or tertiary C₁ to C₆ alkylamino; R⁴ is a primary, secondary or tertiary C₄ to C₈ arylamino; R⁴ is a C₂ to C₆ alkylcarboxylic acid; R⁴ is a C₁ to C₆ alkylester; R⁴ is a branched C₁ to C₆ alkylester; R⁴ is a C₄ to C₈ aryl; R⁴is a C₄ to C₈ arylcarboxylic acid; R⁴ is a C₄ to C₈ arylester; R⁴ is C₄ to C₈ aryl substituted C₁ to C₆ alkyl; R⁴ is a C₄ to C₈ heterocyclic alkyl or aryl; R⁴is an alkyl-substituted or aryl-substituted C₄ to C₈ heterocyclic alkyl or aryl; R⁴ is substituted; R⁴ is unsubstituted; R⁸ is H; and Z is

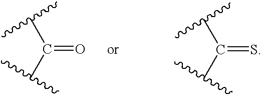

The invention also features: a pharmaceutical composition comprising any of the forgoing compounds and a pharmaceutically acceptable carrier; a method for treating inflammation comprising administering a composition comprising any of the forgoing compounds; a method for treating anxiety comprising administering any of the forgoing compounds; a method for treating a sleep disorder comprising administering any of the forgoing compounds; and a method for treating a respiratory disorder (e.g., asthma) comprising administering any of the forgoing compounds.

The invention features a method for inhibiting COX-2 activity in a patient, the method comprising administering any of the forgoing compounds (e.g., a compound of Formula I wherein X¹ is O and R⁴ is H).

The invention features a method for inhibiting FAAH activity in a patient, the method comprising administering any of the forgoing compounds (e.g., a compound of Formula I wherein X¹ is O and R⁴ is other than H).

The invention features a method for modulating CRTH2 activity on a patient, the method comprising administering any of the forgoing compounds.

In the case of 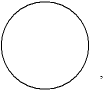
suitable 5-membered ring heterocycles include:
thiophene, furan, and pyrrole 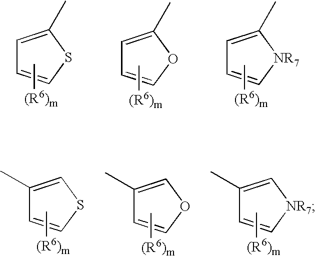
thiazole, oxazole, and imidazole 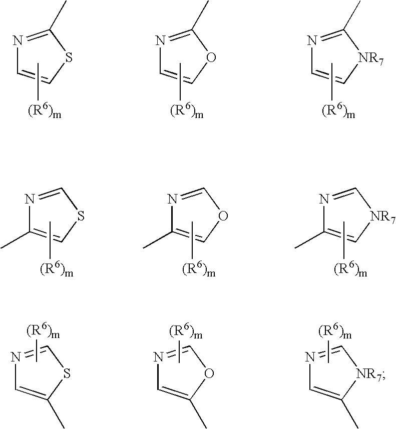
isothiazole, isoxazole, and pyrazole

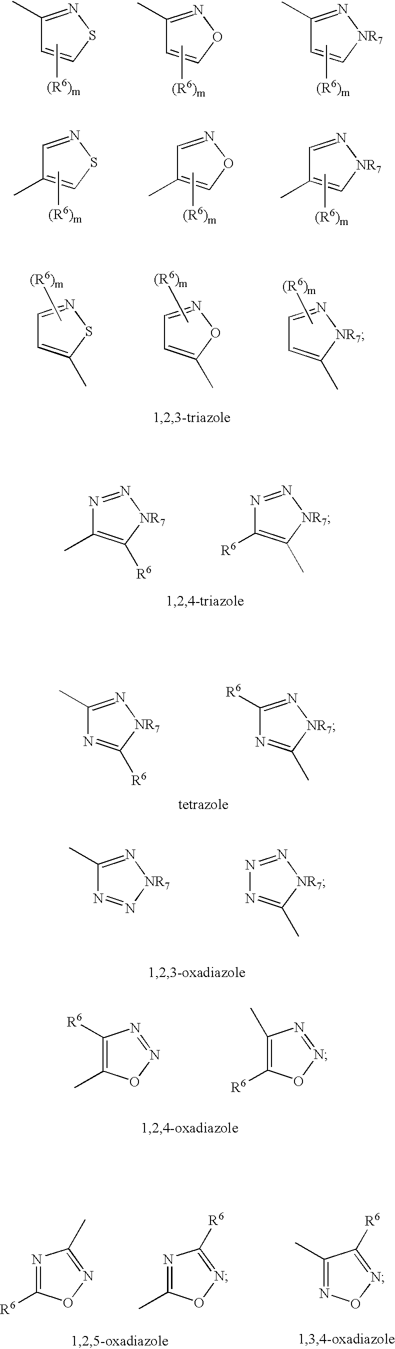

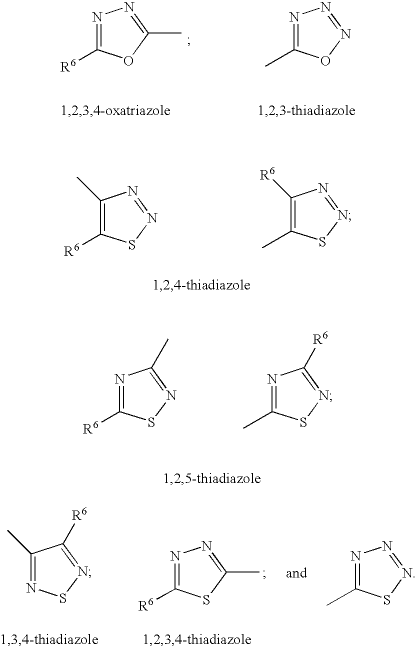

In the case of 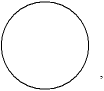
suitable 5 and 6-membered ring saturated heterocycles include:
Piperidine and substituted piperidine 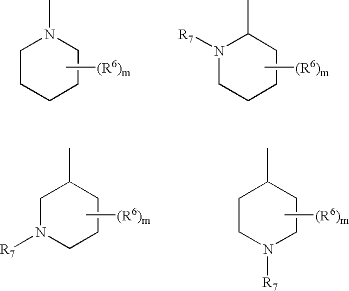
Pyyrolidine and substituted pyrrolidine 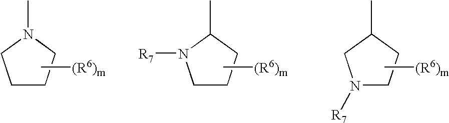
Azetidine and substituted azetidine 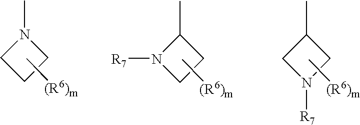
Piperazine and substituted piperazine 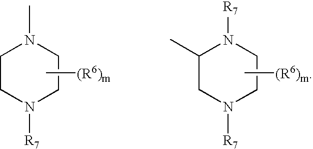
Morpholine and substituted morpholine 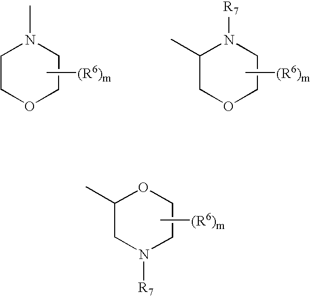
Thiomorpholine and substituted thiomorpholine and their sulfoxide and sulfone derivatives 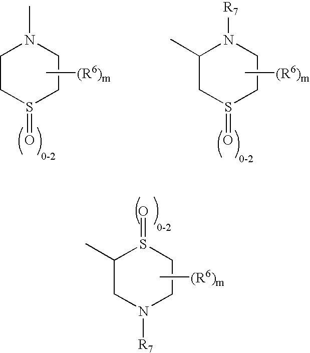
Thioethers, substituted thioethers, their sulfoxides and sulfones 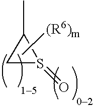
Ethers and substituted ethers 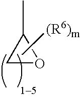
1,4-Thioether-ethers and 1,4-dioxane derivatives 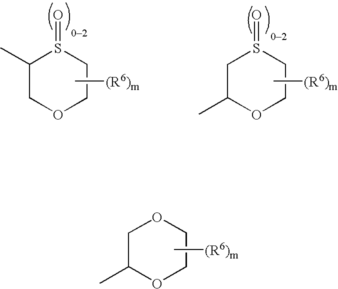
1,4-bis-Thioethers, their sulfoxides and sulfones

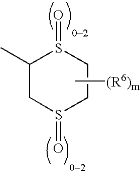

Also included are tetrahydrofuran, dihydrofuran, tetrahydrothiophene, dihydrothiophene, piperidine, dihyropyrrole, 1,3-dithiolane, 1,2-dithiolane, isoxazolidine, isothiazolidine, pyrazolidine, tetrahydro-2H-pyran, tetrahydro-2H-thiopyran, 3,6-dihydro-2H-thiopyran, 3,4-dihydro-2H-thiopyran, piperidine, 1,2,3,6-tetrahydropyridine, 1,2,3,4-tetrahydropyridine, morpholine, thiomorpholine, piperazine, thiomorpholine 1-oxide, thiomorpholine 1,1-dioxide, and the like.

In the case of 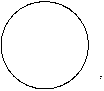
suitable 6-membered ring heteroaryls include:
pyridine

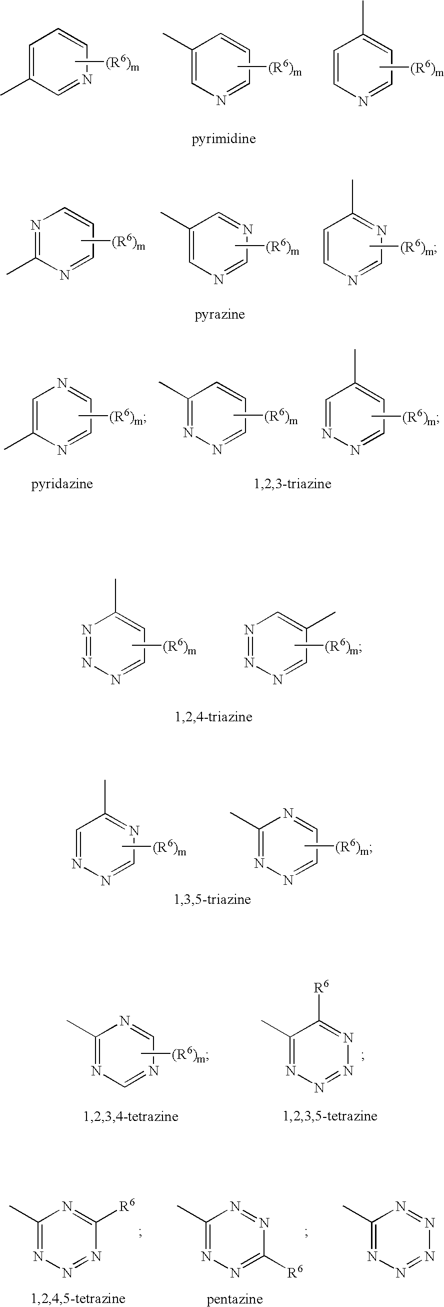

Suitable carbocycles include:
cyclohexyl and substituted cyclohexyl 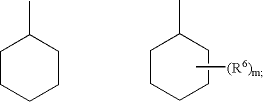
cyclopentyl and substituted cyclopentyl 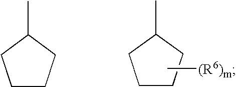
cyclobutyl and substituted cyclobutyl 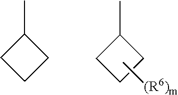
cyclopropyl and substituted cyclopropyl 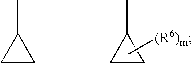
cyclohexenyl and substituted cyclohexenyl 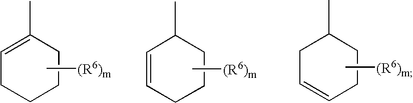
cyclopentenyl and substituted cyclopentenyl 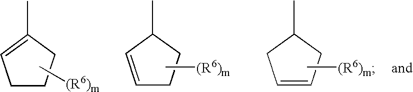
cyclobutenyl and substituted cyclobutenyl

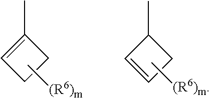

The compounds of the invention inhibit COX-2 or fatty acid amide hydrolase (FAAH) or both COX-2 and FAAH. Some of the compounds of the invention are modulators of CRTH2 activity, e.g., they are either agonists or antagonists of CRTH2. Some compounds may be partial agonists or inverse agonists (inhibitors of basal level activity) of CRTH2. In addition, certain of the compounds of the invention inhibit NPAA.

The compounds of the invention are useful in treating pain and inflammation as well as other disorders such as allergic rhinitis, asthma, atopic dermatitis, eosinophilic esophagitis, and other disorders associated with allergic inflammation.

Some of the compounds of the invention that inhibit COX-2 activity are relatively selective for COX-2 relative to COX-1. Thus, certain COX-2 inhibitors of the invention do not substantially inhibit COX-1 at concentrations at which COX-2 is substantially inhibited. Some of the compounds of the invention that are relatively selective for FAAH do not substantially inhibit COX-2 at concentrations at which FAAH is substantially inhibited. Some compounds are relatively selective for COX-2 as compared to FAAH. These compounds do not substantially inhibit FAAH at concentrations at which COX-2 is substantially inhibited. Other compounds inhibit both COX-2 and FAAH at similar concentrations. These compounds are not particularly selective for COX-2 versus FAAH. Certain compounds of the invention are modulators of CRTH2. Of these compounds, some may also be inhibitors of COX-2 and/or FAAH.

Certain compounds having Formula I or Formula II are COX-2 inhibitors that are selective for inhibition of COX-2 over COX-1 and do not substantially inhibit FAAH. In these compounds R⁴is most often H and X¹ is O.

Certain compounds having Formula I or Formula II are FAAH inhibitors and are selective for inhibition of FAAH over both COX-2 and COX-1. In these compounds R⁴ is most often other than H. In such compounds R⁸ is often a halogen or —CH₃ substituted with one or more F.

Certain compounds having Formula I or Formula II are selective COX-2 inhibitors. In these compounds R⁸ is often H. In some embodiments of Formula I or Formula II, R² is H. Many such compounds are CRTH2 antagonists. Some are not CRTH2 antagonists

In some embodiments of Formula I or Formula II, R is a C₁-C₃ alkyl; optionally independently substituted with one or more halogen. Many such compounds are CRTH2 agonists. Some are not CRTH2 agonists.

Certain compounds having Formula I or Formula II are CRTH2 agonists; in some embodiments the compound has an EC₅₀ for CRTH2 that is less than 20 μM; the compound has an EC₅₀ for CRTH2 that is less than 10 μM; and the compound has an EC₅₀ for CRTH2 that is less than 5 μM.

Certain compounds having Formula I or Formula II are CRTH2 antagonists; in some embodiments the compound has an IC₅₀ for CRTH2 that is less than 20 μM; the compound has an IC₅₀ for CRTH2 that is less than 10 μM; and the compound has an IC₅₀ for CRTH2 that is less than 5 μM.

Certain CRTH2 antagonists have the formula: 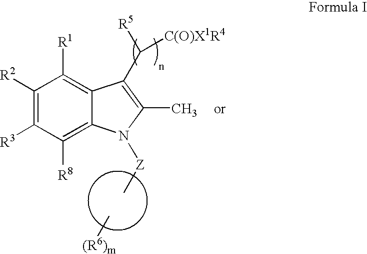
wherein:
R¹ is H or F;
R² is a halogen (e.g., F) or R^{2B}O— wherein R^{2B} is H or CH₃;
R³is H, F, or Cl;
Z is 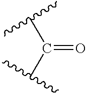
   or C; 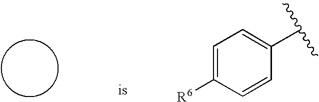
wherein R⁶ is —SCF₃;
R⁸ is H;
R⁵ is H; n is 1;
X¹ is O; and R⁴ is H.

CRTH2 antagonists also include compounds having the formula: 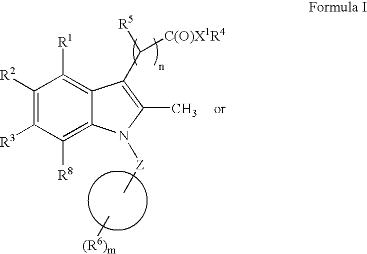
wherein:
R¹ is H or F;
R² is a halogen (e.g., F) or R^{2B}O— wherein R^{2B} is H or CH₃;
R3 is H, F, or Cl;
Z is 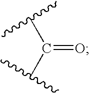 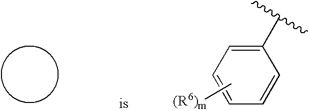
wherein m is 1 and R⁶ at the 3 position is F or Cl or m is 2 and R⁶ at the 3 and 4 positions are both Cl or F;
R⁸ is H;
R⁵ is H; n is 1;
X¹ is O and R⁴ is H.

CRTH2 antagonists also include compounds having the formula: 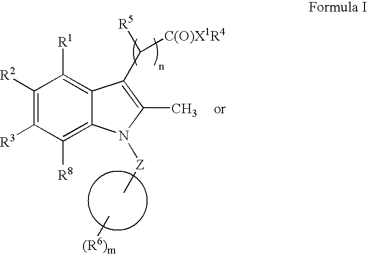
wherein:
R¹ is H or F;
R² is a halogen (e.g., F) or R^{2B}O— wherein R^{2B} is H or CH₃;
R³ is H, F, or Cl;
Z is C; 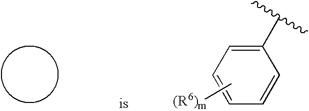
   wherein m is 1, 2, 3, 4, or 5 and R⁶ is F, Cl, Br, or —OCF₃;
R⁸ is H;
R⁵ is H; n is 1; and
X¹ is O and R⁴ is H.

In other embodiments: m is 2 and R⁶ at the 3 and 4 positions are both F or Cl; m is 1 and R⁶ is Cl at the 3 position; m is 1 and R⁶ is Br at the 4 position; m is 1 and R⁶is F at the 4 position; and m is 1 and R⁶ is —OCF₃ at the 4 position.

CRTH2 antagonists also include compounds having the formula: 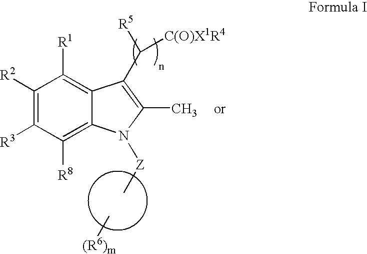
wherein:
R¹ is H or F;
R² is a halogen (e.g., F) or R^{2B}O— wherein R^{2B} is H or CH₃;
R³is H, F, or Cl;
Z is 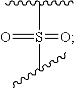 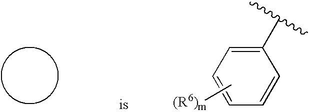
wherein m is 1 and R⁶ at the 3 or 4 position is Cl or F, or m is 2 and R⁶ at both the 3 and 4 positions is Cl or F, or m is 1 and R⁶ at the 4 position in —SCF3, —OCH3 or —OCF₃;
R⁸ is H;
R⁵ is H; n is 1;
X¹ is 0; and R⁴ is H.

CRTH2 agonists include compounds having the formula: 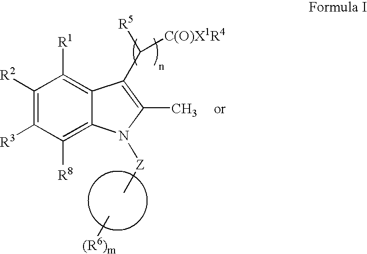
wherein:
R¹ is H or F;
R² is a halogen (e.g., F) or R^{2B}O— wherein R^{2B} is H or CH₃;
R³ is H, F, or Cl;
Z is 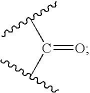 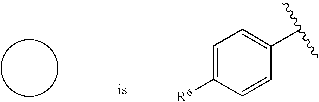
wherein R⁶ is H, F, Cl, —OCH₃, —CH₃;
R⁸ is H;
R⁵ is H; n is 1
X¹ is O; and R⁴ is H.

COX-2 antagonists include compounds having the formula: 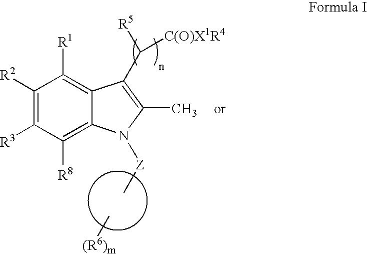
wherein:
R¹ is H or F; R² is a halogen (e.g., F) or R^{2B}O— wherein R^{2B} is H or CH₃; R³ is H, F, or Cl;
Z is 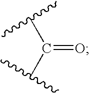 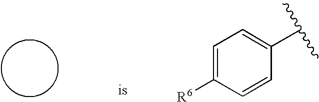
wherein R⁶ is H, F, Cl, —OCH₃, —CH₃;
R⁸ is H,
R⁵ is H; n is 1;
X¹ is O; and R⁴ is H.

FAAH antagonists also include compounds having the formula: 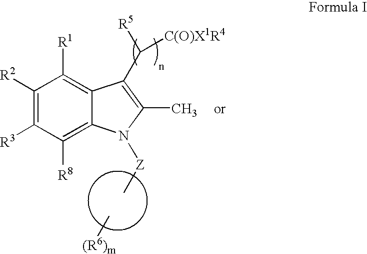
wherein:
R¹ is H or F; R² is a halogen (e.g., F) or R^{2B}O— wherein R^{2B} is H or CH₃; R³ is H, F, or Cl;
Z is 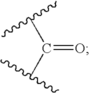 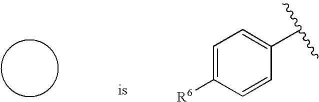
wherein R⁶ is H, F, Cl, —OCH₃, —CH₃;
R⁸ is H,
R⁵ is H; n is 1
X¹ is O or N(H); and R⁴ is a C₁ to C₈ alkyl optionally independently substituted with one or more —OH or —CO₂H.

The invention also features compositions comprising a compound having Formula I or Formula II, wherein the composition contains no more than 0.0001%, 0.001%, 0.01%, 0.1%, 0.3%, 0.5%, 0.9%. 1.9%, 5.0%, or 10% by weight other compounds.

The invention also features a method of treating a disorder associated with unwanted COX-2 activity or unwanted FAAH activity or both unwanted COX-2 activity and unwanted FAAH activity. In some embodiments of the method: the disorder is an inflammatory disorder; and R²O— is a hydroxy group or a group that is metabolized to a hydroxy group, i.e., R²O— is a prodrug of a hydroxy group. In certain embodiments R²O— is an alkoxy group that is not rapidly metabolically converted to a hydroxy group or is not significantly metabolically converted to a hydroxy group. In other embodiments, the invention includes a therapeutic method comprising administering a compound of the invention together with an agent for the treatment of inflammation, pain or fever, e.g., a NSAID.

The invention also features a compound having Formula I or Formula II wherein the prodrug of a hydroxy moiety is selected from: (a) an ester having a C₁ to C₆ branched or straight chain alkyl group, (b) phosphate ester having C₁ to C₆ branched or straight chain alkyl groups, (c) a carbamate having C₁ to C₆ branched or straight chain alkyl groups, and (d) a carbonate group having a C₁ to C₆ branched or straight chain alkyl group.

The invention also features: a method for treating pain comprising administering a compound of the invention or a pharmaceutical composition comprising the compound; a method for treating inflammation comprising administering a compound of the invention or a pharmaceutical composition comprising the compound; a method for treating both pain and/or inflammation comprising administering a compound of the invention or a pharmaceutical composition comprising the compound; a method for treating anxiety comprising administering a compound of the invention or a pharmaceutical composition comprising the compound; and a method for treating a sleep disorder comprising administering a compound of the invention or a pharmaceutical composition comprising the compound.

The invention includes: a method for lowering COX-2 activity in a patient by administering the compound or a pharmaceutical composition comprising the compound; a method for lowering FAAH activity in a patient by administering the compound or a pharmaceutical composition comprising the compound; and a method for lowering both FAAH activity and COX-2 activity in a patient by administering the compound or a pharmaceutical composition comprising the compound. In various embodiments administration of the compound or a composition comprising the compound does not lower COX-1 activity by more than 5% at a dosage that decreases COX-2 activity by at least 25%.

The invention also includes a method for treating a disorder characterized by imbalance of the Th1/Th2 ratio towards Th1, the method comprising administering a compound having Formula I or Formula II. In certain embodiments, the disorder is selected from: rheumatoid arthritis, Type I diabetes, psoriasis, gastritis, irritable bowel disorder, multiple sclerosis, painless throiditis, lupus, and Crohn's Disease.

The invention also includes a method for treating a disorder characterized by imbalance of the Th1/Th2 ratio towards Th2, the method comprising administering a compound having Formula I or Formula II. In certain embodiments, the disorder is selected from: asthma, atopic dermatitis, allergic rhinitis, allergy, and Grave's Disease.

The invention features a method for treating a disorder selected from asthma, allergic rhinitis, atopic dermatitis, eosinophilic esophagitis, and other disorders associated with allergic inflammation, the method comprising administering a compound having Formula I or Formula II. In some embodiments, the compound is a CRTH2 atagonists. In certain embodiments, R² is R^{2B}O— and R^{2B} is H. In some embodiments, the method further comprises administering a second compound that is an anti-inflammatory agent. The invention also features a method for treating a disorder characterized by undesirable activation of Th1 cells, the method comprising administering compound of Formula I or Formula II. The invention also features a method for treating a disorder characterized by undesirable activation of Th2 cells, the method comprising administering compound of Formula I or Formula II.

In some embodiments, the disorder is selected from: rheumatoid arthritis, Type I diabetes, psoriasis, gastritis, irritable bowel disorder, multiple sclerosis, painless thyroiditis, lupus, and Crohn's Disease. In other embodiments, the disorder is selected from: asthma, atopic dermatitis, allergic rhinitis, allergy, and Grave's Disease. The invention also features a method for modulating CRTH2 activity in a patient, the method comprising administering a compound having Formula I or Formula II to a patient. In some embodiments, the compound is a CRTH2 agonist. In others it is an antagonist. In some embodiments, R² is R^{2B}O— and R^{2B} is H. In others R^{2B} is a C₁-C₃ alkyl, optionally independently substituted with one or more halogen.

The invention features a pharmaceutical composition comprising a compound of the invention (or a salt thereof, e.g., a TRIS or other salt thereof) and a pharmaceutically acceptable carrier.

The invention also features a method for treating a patient for a disorder characterized by an increased level of a cytokine produced by Th2 cells, e.g., a disorder characterized by increased (e.g., undesirably increased) IL-4, IL-10 and/or IL-13 in a patient, the method comprising administering to the patient a CRTH2 modulator described herein. The invention also features a method for treating a patient for a disorder characterized by an increased level of a cytokine produced by Th1, e.g., a disorder characterized by increased (e.g., undesirably increased) interferon-,y in a patient, the method comprising administering to the patient a CRTH2 modulator described herein. The invention also features a method for decreasing the Th1 cell/Th2 cell ratio in a patient, the method comprising administering to the patient a CRTH2 modulator, e.g., a CRTH2 agonist.

The invention also features a method for increasing the Th1 cell/Th2 cell ratio in a patient, the method comprising administering to the patient a CRTH2 modulator, e.g., a CRTH2 antagonist.

In some embodiments the CRTH2 modulators are also inhibitors of cyclooxygenase-1 (COX-1) and/or cyclooxygenase-2 (COX-2). Among compounds that inhibit COX-2 and/or COX-1, those that are those that selective for COX-2 are preferred. Thus, in some embodiments: the compound exhibits an IC₅₀ for COX-2 that is at least 20,000; 10,000; 1,000; 500; 100; 50; or 25 μM, and have an IC₅₀ for COX-1 that is even greater than the IC₅₀ for COX-2. In some embodiments the COX-1 IC₅₀ for a compound is at least 2, 5, 10, 25, 50, 100, 500, 1000 or more times the COX-1 IC₅₀ for indomethacin in the same assay.

Some desirable compound having the structure of Formula I or Formula II have an EC₅₀ for human CRTH2 that is less than 20, 10, 2.0, 1.5, 1.0, 0.5, 0.4, 0.3, 0.2, 0.1, 0.08, 0.06, 0.04, 0.02, or 0.01 μM.

Some desirable compound having the structure of Formula I or Formula II have an IC₅₀ for human CRTH2 that is less than 20, 10, 2.0, 1.5, 1.0, 0.5, 0.4, 0.3, 0.2, 0.1, 0.08, 0.06, 0.04, 0.02, or 0.01 μM.

In some embodiments of the invention, the composition is administered to a patient that is not being treated with a non-selective NSAID, e.g., a patient that is not being treated with indomethacin.

In certain embodiments the compounds are administered in combination with a second compound useful for reducing inflammation or pain.

The subject can be a mammal, preferably a human. Identifying a subject in need of such treatment can be in the judgment of a subject or a health care professional and can be subjective (e.g., opinion) or objective (e.g., measurable by a test or diagnostic method).

The term “treating” or “treated” refers to administering a compound described herein to a subject with the purpose to cure, heal, alleviate, relieve, alter, remedy, ameliorate, improve, or affect a disease, the symptoms of the disease or the predisposition toward the disease.

“An effective amount” refers to an amount of a compound that confers a therapeutic effect on the treated subject. The therapeutic effect may be objective (i.e., measurable by some test or marker) or subjective (i.e., subject gives an indication of or feels an effect). An effective amount of the compound described above may range from about 0.05 mg/Kg to about 500 mg/Kg, alternatively from about 1 to about 50 mg/Kg. Effective doses will also vary depending on route of administration, as well as the possibility of co-usage with other agents.

The term “mammal” includes, for example, mice, hamsters, rats, cows, sheep, pigs, goats, and horses, monkeys, dogs (e.g., Canis familiaris), cats, rabbits, guinea pigs, and primates, including humans.

The term “prodrug” refers to compounds which are drug precursors which, following administration and absorption, release the drug in vivo through a metabolic process. Exemplary prodrugs include acyl amides of the amino compounds of this invention such as amides of alkanoic (C₁ to C₆)acids, amides of aryl acids (e.g., benzoic acid) and alkane (C₁ to C₆ )dioic acids.

The invention includes prodrugs that are converted in vivo so that R^{2B}O— becomes a hydroxyl group. Thus, in the prodrug form of the compounds of the invention R^{2B}O— is a group that is converted to a hydroxyl group. For example, in a prodrug form of the compounds of the invention, R^{2B}O— can be a carbonate, ester, carbamate, phosphate ester or a similar group.

Thus, R^{2B} can be, for example, 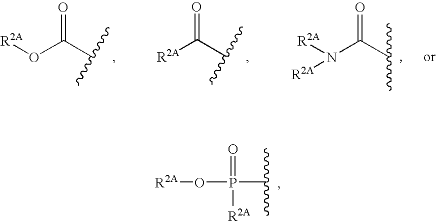
wherein each R^{2A} is independently: H or a C₁ to C₆ alkyl, alkenyl, alkynyl, aryl, cycloalkyl, or arylalkyl optionally independently substituted with one or more halogen.

Particularly useful are compound in which R^{2A} is selected from: H and a substituted or unsubstituted C₁ alkyl, a C₂ alkyl, a C₃ alkyl or a C₄ alkyl.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it (i.e., 1, 2, 3, 4, 5, 6, 7, 8, 9, 10, 11, 12). The term “haloalkyl” refers to an alkyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. Examples of “arylalkyl” or “aralkyl” include benzyl and 9-fluorenyl groups.

The terms “alkylamino” and “dialkylamino” refer to —NH(alkyl) and —N(alkyl)₂ radicals respectively. The term “aralkylamino” refers to a —NH(aralkyl) radical. The term “alkoxy” refers to an —O-alkyl radical. The term “mercapto” refers to an SH radical. The term “thioalkoxy” refers to an —S-alkyl radical.

The term “aryl” refers to an aromatic monocyclic, bicyclic, or tricyclic hydrocarbon ring system, wherein any ring atom capable of substitution can be substituted by a substituent.

Examples of aryl moieties include, but are not limited to, phenyl, naphthyl, and anthracenyl.

The term “cycloalkyl” as employed herein includes saturated monocyclic, bicyclic, tricyclic, or polycyclic hydrocarbon groups having 3 to 12 carbons, wherein any ring atom capable of substitution can be substituted by a substituent. Examples of cycloalkyl moieties include, but are not limited to, cyclopentyl, norbornyl, and adamantyl.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “oxo” refers to an oxygen atom, which forms a carbonyl when attached to carbon, an N-oxide when attached to nitrogen, and a sulfoxide or sulfone when attached to sulfur.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, alkenyl, alkynyl, alkoxy, acyloxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)n alkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

The invention includes salts, particularly physiologically acceptable salts, and solvates of the compounds having Formula I or Formula II. Solvates are forms of the compounds in which the compound forms a complex with solvent molecules by coordination in the solid or liquid states. Hydrates are a special form of solvate in which the compound is coordinated with water.

Certain compounds having Formula I or Formula II may exist in stereoisomeric forms such as enantiomers, diastereomers and mixtures thereof. Mixtures can be separated into stereoisomerically pure constituents.

Certain compounds having Formula I or Formula II may be tautomeric, and the invention encompasses the various tautomeric mixtures.

The details of one or more embodiments of the invention are set forth in the description below. Other features, objects, and advantages of the invention will be apparent from the description and drawings, and from the claims. The patents, patent applications, and publications referenced herein are hereby incorporated by reference in their entirety.

### DESCRIPTION OF DRAWINGS

FIG. 1 is a table that provides COX-1 IC₅₀ (purified enzyme assay) and COX-2 IC₅₀ (purified enzyme assay) for a number of compounds. All numbers are in μm units.

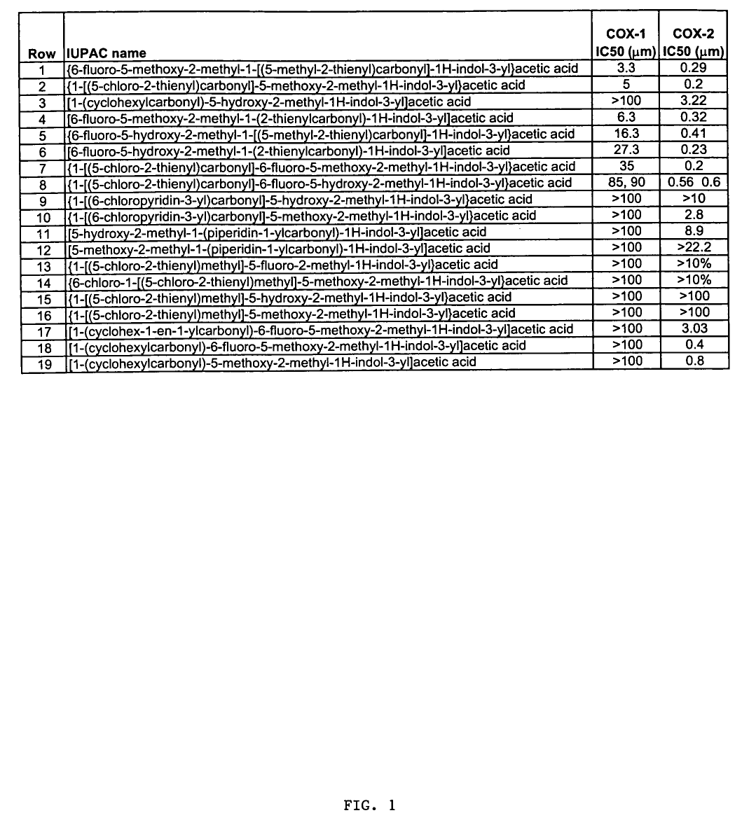

FIG. 2a is a table that provides CRTH2 activity data for a number of compounds which are CRTH2 agonists. Compounds were tested for CRTH2 agonist activity at 10 and 1 μM.

FIG. 2b is a table that provides CRTH2 activity data for a number of compounds, some of which are CRTH2 antagonists. Compounds were tested for CRTH2 antagonist activity at 10 μM.

### DETAILED DESCRIPTION

The invention features compounds that inhibit COX-2 FAAH, and/or modulators of CRTH2. Certain COX-2 inhibitors are selective COX-2 inhibitors in that they are selective for inhibition of COX-2 as compared to COX-1. Certain of the FAAH inhibitors are selective for inhibition of FAAH relative to both COX-2 and COX-1. Certain of the COX-2 inhibitors, in addition to being selective for COX-2 relative to COX-1, are selective for COX-2 relative to FAAH. Certain compounds of the invention are modulators of CRTH2. Of these compounds, some may also be inhibitors of COX-2 and/or FAAH.

Certain compounds of the invention are expected to have an increased half-life in the human body compared to certain structurally related compounds. Certain compounds of the invention are expected to have reduced renal and/or gastric toxicity compared to certain structurally related compounds.

Useful selective COX-2 inhibitors are those which inhibit COX-2 activity at physiological concentrations where COX-1 activity is not significantly inhibited. Thus, the compounds have an IC₅₀ for COX-1 that is at least 2-, 5-, 10-, 15-, 20-, 100-, 500-, 1,000-fold greater than the IC₅₀ for COX-2. Certain compounds do not significantly inhibit COX-1 at a therapeutically effective concentration, e.g., a concentration effective to reduce pain or inflammation attributable to COX-2 associated prostaglandin production. Useful compounds include those having an IC₅₀ for COX-2 of less than about 2.0, 1.5, 1.0, 0.5, 0.4, 0.3, 0.2, 0.1, 0.08, 0.06, 0.04, 0.02, or 0.01 μM, and have an IC₅₀ for COX-1 of greater than about 1, 5, 10, 15, 20, 40 or 100 μM. In certain embodiments the COX-2 IC₅₀ for a compound is less than 20, 10, 5, 3, 2, 1, 0.5, 0.4, 0.3, 0.2, 0.1 or 0.05 times the COX-2 IC₅₀ for indomethacin in the same assay. In certain embodiments the COX-1 IC₅₀ for a compound is at least 2, 5, 10, 25, 50, 100, 500, 1000 or more times the COX-1 IC₅₀ for indomethacin in the same assay. In certain embodiments, the selectivity for COX-2 over COX-1 for a compound is greater than 3, 5, 10, 50, 100, 200, 500 or 1000 times the selectivity of indomethacin in the same assays.

Certain useful selective FAAH inhibitors include those which inhibit FAAH activity at a physiological concentration at which COX-1 and COX-2 activity is not significantly inhibited. Thus, the compounds have an IC₅₀ for COX-1 and COX-2 that is at least 2-, 5-, 10-, 15-, 20-, 100-, 500-, 1,000-fold greater than the IC₅₀ for FAAH. Particularly desirable are compounds that do not measurably inhibit COX-1 and COX-2 at a therapeutically effective concentration, e.g., a concentration effective to reduce pain. Useful compounds include those having an IC₅₀ for FAAH of less than about 80, 60, 40, 20, 10, 5, 2.0, 1.5, 1.0, 0.5, 0.4, 0.3, 0.2, 0.1, 0.08, 0.06, 0.04, 0.02, or 0.01 μM, and have an IC₅₀ for COX-1 and COX-2 of greater than about 1, 5, 10, 15, 20, 50, 100, 200, or 400 μM. In certain embodiments, the IC₅₀ for FAAH for a compound is no more than about 5, 1, 0.1, 0.05, 0.01 or 0.001 times the IC₅₀ for FAAH of indomethacin in the same assay.

Of course, other useful FAAH inhibitors also inhibit COX-2 at physiological concentrations at which COX-1 activity is not significantly inhibited. Particularly desirable are compounds that do not measurably inhibit COX-1 at a therapeutically effective concentration, e.g., a concentration effective to reduce pain. Useful compounds include those having an IC₅₀ for FAAH of less than about 80, 60, 40, 20, 10, 5, 2.0, 1.5, 1.0, 0.5, 0.4, 0.3, 0.2, 0.1, 0.08, 0.06, 0.04, 0.02, or 0.01 μM, an IC50 for COX-2 of less than about 2.0, 1.5, 1.0, 0.5, 0.4, 0.3, 0.2, 0.1, 0.08, 0.06, 0.04, 0.02, or 0.01 μM, and an IC₅₀ for COX-1 of greater than about 1, 5, 10, 15, or 20 RM. In certain embodiments the COX-2 IC₅₀ for such a FAAH inhibitor is less than 20, 10, 5, 3, 2, 1, 0.5, 0.4, 0.3, 0.2, 0.1 or 0.05 times the COX-2 IC₅₀ for indomethacin in the same assay. In certain embodiments the COX-1 IC₅₀ for such a FAAH inhibitor is at least 2, 5, 10, 25, 50, 100, 500, 1000 or more times the COX-1 IC₅₀ for indomethacin in the same assay.

Certain useful selective COX-2 inhibitors include those which inhibit COX-2 activity at physiological concentrations where FAAH activity is not significantly inhibited. Particularly desirable are compounds that do not significantly inhibit FAAH at a therapeutically effective concentration, e.g., a concentration effective to reduce pain. Useful compounds include those having an IC₅₀ for COX-2 of less than about 2.0, 1.5, 1.0, 0.5, 0.4, 0.3, 0.2, 0.1, 0.08, 0.06, 0.04, 0.02, or 0.01 μM, and have an IC₅₀ for FAAH of greater than about 5, 10, 15, 20, 50, 100, 200 or 400 μM. Of course, other useful COX-2 inhibitors also inhibit FAAH at therapeutically relevant doses, i.e., they are not particularly selective for COX-2 over FAAH. In certain embodiments the COX-2 IC₅₀ for a compound is less than 20, 10, 5, 3, 2, 1, 0.5, 0.4, 0.3, 0.2, 0.1 or 0.05 times the COX-2 IC₅₀ for indomethacin in the same assay. In certain embodiments the COX-1 IC₅₀ for a compound is at least 2, 5, 10, 25, 50, 100, 500, 1000 or more times the COX-1 IC₅₀ for indomethacin in the same assay.

Certain compounds having Formula I or Formula II, e.g., those in which X¹ is —O— and R⁴ is a C₁ to C₆ alkyl are effective FAAH inhibitors, but are not particularly effective COX-2 inhibitors. However, many such compounds are metabolized to a form in which X¹ is —O— and R⁴ is H. Many of these metabolites are effective COX-2 inhibitors, but are not highly effective FAAH inhibitors, although they can inhibit FAAH to some extent. Thus, compounds having Formula I or Formula II which are FAAH inhibitors and in which X¹ is —O— and R⁴ is a C₁ to C₆ alkyl can exhibit two different phases of activity when administered to a patient—an initial, relatively high FAAH inhibition phase characterized by little or no significant COX-2 inhibition followed by a COX-2 inhibition phase characterized by reduced FAAH inhibition.

### EXAMPLES

Certain useful compounds are described below.

### {1-[(5-chlorothien-2-yl)carbonyl]-5-hydroxy-2-methyl-1H-indol-3-yl}acetic acid

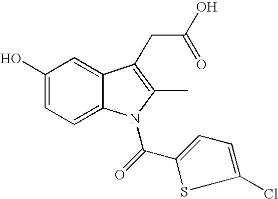

mp 195° C.

¹H NMR (CDCl₃/300 MHz) 7.43 (d, 1H, J=4.2 Hz), 7.13-7.10 (m, 2H), 6.87 (d, 1H, J=2.1 Hz), 6.61 (dd, 1H, J=8.7, 2.1 Hz), 3.66 (s, 2H), 2.38 (s, 3H).

### {1-[(5-chlorothien-2-yl)carbonyl]-6-fluoro-5-methoxy-2-methyl-1H-indol-3-yl}acetic acid

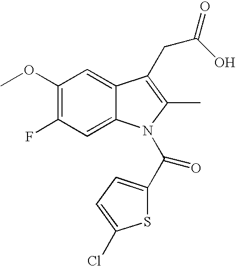

mp 169° C.

¹H NMR (CDCl₃/300 MHz) 7.35 (d, 1H, J=4.0 Hz), 7.09 (d, 1H, J=11.7 Hz), 7.00 (d, 1H, J=7.2 Hz), 6.98 (d, 1H, J=4.0 Hz), 3.93 (s, 3H), 3.70 (s, 2H), 2.42 (s, 3H).

### {1-[(5-chlorothien-2-yl)carbonyl]-6-fluoro-5-hydroxy-2-methyl-1H-indol-3-yl}acetic acid

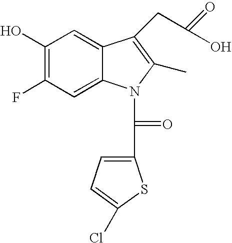

mp 174° C.

¹H NMR (CDCl₃/300 MHz) 7.34 (d, 1H, J=3.9 Hz), 7.13 (d, 1H, J=11.1 Hz), 7.07 (d, 1H, J=8.4 Hz), 6.98 (d, 1H, J=3.9 Hz), 3.66 (s, 2H), 2.39 (s, 3H).

### [6-fluoro-5-methoxy-2-methyl-1-(thien-2-ylcarbonyl)-1H-indol-3-yl]acetic acid

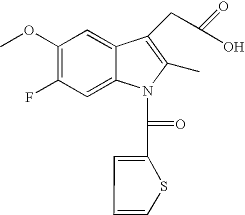

mp 137° C.

¹H NMR (CDCl₃/300 MHz) 7.77 (dd, 1H, J=5.0, 1.2 Hz), 7.54 (dd, 1H, J=3.9, 1.2 Hz), 7.15 (dd, 1H, J=5.0, 3.9 Hz), 7.01 (d, 1H, J=12.0 Hz), 7.00 (d, 1H, J=8.1 Hz), 3.92 (s, 3H), 3.69 (s, 2H), 2.41 (s, 3H).

### {6-fluoro-5-methoxy-2-methyl-1-[(5-methylthien-2-yl)carbonyl]-1H-indol-3-yl}acetic acid

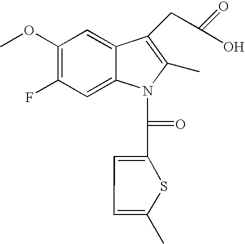

mp 152° C.

¹H NMR (CDCl₃/300 MHz) 7.35 (d, 1H, J=3.9 Hz), 7.06 (d, 1H, J=12.3), 6.99 (d, 1H, J=8.1 Hz), 6.81 (d, 1H, J=3.9 Hz), 3.92 (s, 3H), 3.68 (s, 2H), 2.60 (s, 3H), 2.42 (s, 3H).

### {6-fluoro-5-hydroxy-2-methyl-1-[(5-methylthien-2-yl)carbonyl]-1H-indol-3-yl}acetic acid

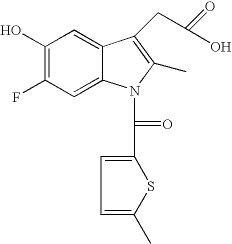

mp 197° C.

¹H NMR (CD₃OD/300 MHz) 7.40 (d, 1H, J=4.0 Hz), 6.99 (d, 1H, J=8.7 Hz), 6.98 (d, 1H, J=11.7 Hz), 6.93 (d, 1H, J=4.0 Hz), 3.64 (s, 2H), 2.62 (s, 3H), 2.34 (s, 3H).

### [6-fluoro-5-hydroxy-2-methyl-1-(thien-2-ylcarbonyl)-1H-indol-3-yl] acetic acid

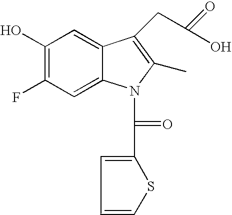

mp 219° C.

¹H NMR (CD₃OD/300 MHz) 7.97 (dd, 1H, J=5.1, 1.2 Hz), 7.59 (dd, 1H, J=3.9, 1.2 Hz), 7.22 (dd, 1H, J=5.1, 3.9 Hz), 7.00 (d, 1H, J=8.7 Hz), 6.94 (d, 1H, J=12.0 Hz), 3.65 (s, 2H), 2.32 (s, 3H).

### [1-(cyclohexylcarbonyl)-5-methoxy-2-methyl-1H-indol-3-yl]acetic acid

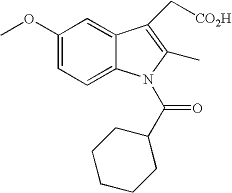

mp 129° C.

¹H NMR (CDCl₃/300 MHz) 7.62 (d, 1H, J=9.0 Hz), 6.93 (d, 1H, J=2.7), 6.86 (dd, 1H, J=9.0, 2.7 Hz), 3.85 (s, 3H), 3.67 (s, 2H), 3.18 (m, 1H), 2.04-1.32 (m, 10H).

### [1-clohexylcarbonyl)-5-hydroxy-2-methyl-1H-indol-3-yl]acetic acid

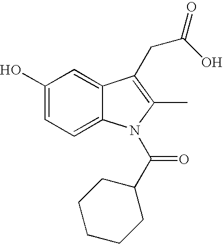

¹H NMR (CDCl₃/300 MHz) 7.50 (d, 1H, J=9.0 Hz), 6.95 (d, 1H, J=2.1), 6.73 (dd, 1H, J=9.0, 2.1 Hz), 3.53 (s, 2H), 3.12 (m, 1H), 2.49 (s, 3H), 2.00-1.05 (m, 10H).

### {1-[(6-chloropyridin-3-yl)carbonyl]-5-methoxy-2-methyl-1H-indol-3-yl}acetic acid

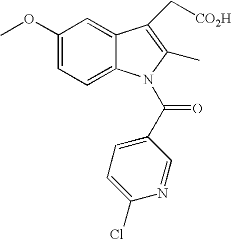

mp 153° C.

¹H NMR (CDCl₃/300 MHz) 8.71 (d, 1H, J=2.7 Hz), 8.27 (dd, 1H, J=8.1, 2.7 Hz), 7.98 (dd, 1H, J=8.1, 2.7 Hz), 7.48 (d, 1H, J=8.7 Hz), 6.97 (d, 1H, J=2.4 Hz), 6.76 (dd, 1H, J=8.7, 2.4 Hz), 3.84 (s, 3H), 3.71 (s, 2H), 2.41 (s, 3H).

### [1-(cyclohexylcarbonyl)-6-fluoro-5-methoxy-2-methyl-1H-indol-3-yl]acetic acid

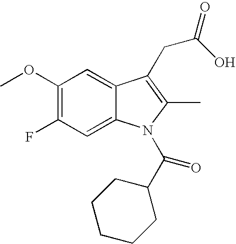

mp 104° C.

¹H NMR (CDCl₃/300 MHz) 7.72 (d, 1H, J=12.9 Hz), 7.13 (d, 1H, J=8.1), 3.91 (s, 3H), 3.69 (s, 2H), 3.23 (m, 1H), 2.56 (s, 3H), 2.05-1.27 (m, 10H).

### [5-methoxy-2-methyl-1-(piperidin-1-ylcarbonyl)-1H-indol-3-yl]acetic acid

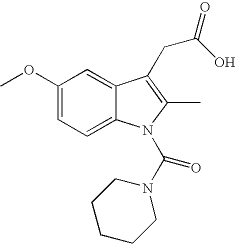

yellow oil

¹H NMR (CDCl₃/300 MHz) 7.16 (d, 1H, J=9.0 Hz), 6.96 (d, 1H, J=2.7), 6.81 (dd, 1H, J=9.0, 2.7 Hz), 3.83 (s, 3H), 3.66 (s, 2H), 3.58-3.30 (m, 4H), 2.40 (s, 3H), 1.70-1.55 (m, 6H).

### [5-hydroxy-2-methyl-1-(piperidin-1-ylcarbonyl)-1H-indol-3-yl]acetic acid

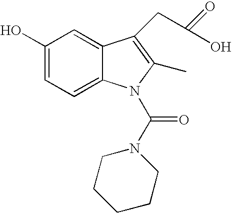

mp 235° C.

¹H NMR (CDCl₃/300 MHz) 6.99 (d, 1H, J=8.7 Hz), 6.79 (s, 1H), 6.64 (d, 1H, J=8.7 Hz), 3.47 (s, 2H), 3.47-3.30 (m, 4H), 2.33 (s, 3H), 1.72-1.43 (m, 6H).

Additional compounds include:
[6-fluoro-5-hydroxy-2-methyl-1-(thien-2-ylcarbonyl)-1H-indol-3-yl]acetic acid derivatives having the formula: 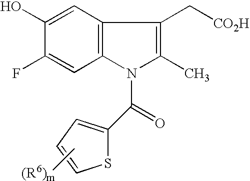
   including:
[6-fluoro-5-hydroxy-2-methyl-1-(thien-2-ylcarbonyl)-1H-indol-3-yl]acetic acid;
{6-fluoro-1-[(5-fluorothien-2-yl)carbonyl]-5-hydroxy-2-methyl-1H-indol-3-yl}acetic acid;
{1-[(5-chlorothien-2-yl)carbonyl]-6-fluoro-5-hydroxy-2-methyl-1H-indol-3-yl}acetic acid;
{1-[(5-bromothien-2-yl)carbonyl]-6-fluoro-5-hydroxy-2-methyl-1H-indol-3-yl}acetic acid;
{6-fluoro-5-hydroxy-1-[(5-hydroxythien-2-yl)carbonyl]-2-methyl-1H-indol-3-yl}acetic acid;
{6-fluoro-5-hydroxy-1-[(5-methoxythien-2-yl)carbonyl]-2-methyl-1H-indol-3-yl}acetic acid;
{1-[(5-ethoxythien-2-yl)carbonyl]-6-fluoro-5-hydroxy-2-methyl-1H-indol-3-yl}acetic acid;
(1-{[5-(difluoromethoxy)thien-2-yl]carbonyl}-6-fluoro-5-hydroxy-2-methyl-1H-indol-3-yl)acetic acid;
(6-fluoro-5-hydroxy-2-methyl-1-{[5-(trifluoromethoxy)thien-2-yl]carbonyl)}-H-indol-3-yl)acetic acid;
(6-fluoro-5-hydroxy-2-methyl-1-{[5-(pentafluoroethoxy)thien-2-yl]carbonyl}-1H-indol-3-yl)acetic acid;
(6-fluoro-5-hydroxy-2-methyl-1-{[5-(1,1,2,2-tetrafluoroethoxy)thien-2-yl]carbonyl}-1H-indol-3-yl)acetic acid;
{6-fluoro-5-hydroxy-2-methyl-1-[(5-methylthien-2-yl)carbonyl]-1H-indol-3-yl}acetic acid;
(1-{[5-(difluoromethyl)thien-2-yl]carbonyl}-6-fluoro-5-hydroxy-2-methyl-1H-indol-3-yl)acetic acid;
(6-fluoro-5-hydroxy-2-methyl-1-{[5-(trifluoromethyl)thien-2-yl]carbonyl}-1H-indol-3-yl)acetic acid;
(6-fluoro-5-hydroxy-2-methyl-1-{[5-(methylthio)thien-2-yl]carbonyl}-1H-indol-3-yl)acetic acid;
[1-({5-[(difluoromethyl)thio]thien-2-yl}carbonyl)-6-fluoro-5-hydroxy-2-methyl-1H-indol-3-yl]acetic acid;
[6-fluoro-5-hydroxy-2-methyl-1-({5-[(trifluoromethyl)thio]thien-2-yl}carbonyl)-1H-indol-3-yl]acetic acid;
[6-fluoro-5-hydroxy-2-methyl-1-({5-[(pentafluoroethyl)thio]thien-2-yl}carbonyl)-1H-indol-3-yl]acetic acid;
[6-fluoro-5-hydroxy-2-methyl-1-({5-[(1,1,2,2-tetrafluoroethyl)thio]thien-2-yl}carbonyl)-1H-indol-3-yl]acetic acid; and
{1-[(5-cyanothien-2-yl)carbonyl]-6-fluoro-5-hydroxy-2-methyl-1H-indol-3-yl}acetic acid

[6-fluoro-5-hydroxy-2-methyl-1-(thien-3-ylcarbonyl)-1H-indol-3-yl]acetic acid derivatives having the formula: 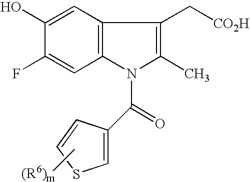
including:
[6-fluoro-5-hydroxy-2-methyl-1-(thien-3-ylcarbonyl)-1H-indol-3-yl]acetic acid;
{6-fluoro-1-[(5-fluorothien-3-yl)carbonyl]-5-hydroxy-2-methyl-1H-indol-3-yl}acetic acid;
{1-[(5-chlorothien-3-yl)carbonyl]-6-fluoro-5-hydroxy-2-methyl-1H-indol-3-yl}acetic acid;
{1-[(5-bromothien-3-yl)carbonyl]-6-fluoro-5-hydroxy-2-methyl-1H-indol-3-yl}acetic acid;
{6-fluoro-5-hydroxy-1-[(5-hydroxythien-3-yl)carbonyl]-2-methyl-1H-indol-3-yl}acetic acid;
{6-fluoro-5-hydroxy-1-[(5-methoxythien-3-yl)carbonyl]-2-methyl-1H-indol-3-yl}acetic acid;
{1-[(5-ethoxythien-3-yl)carbonyl]-6-fluoro-5-hydroxy-2-methyl-1H-indol-3-yl}acetic acid;
(1-{[5-(difluoromethoxy)thien-3-yl]carbonyl}-6-fluoro-5-hydroxy-2-methyl-1H-indol-3-yl)acetic acid;
(6-fluoro-5-hydroxy-2-methyl-1-{[5-(trifluoromethoxy)thien-3-yl]carbonyl}-1H-inol-3-yl)acetic acid;
(6-fluoro-5-hydroxy-2-methyl-1-{[5-(pentafluoroethoxy)thien-3-yl]carbonyl}-1H-indol-3-yl)acetic acid;
(6-fluoro-5-hydroxy-2-methyl-1-{[5-(1,1,2,2-tetrafluoroethoxy)thien-3-yl]carbonyl}-1H-indol-3-yl)acetic acid;
{6-fluoro-5-hydroxy-2-methyl-1-[(5-methylthien-3-yl)carbonyl]-1H-indol-3-yl}acetic acid;
(1-{[5-(difluoromethyl)thien-3-yl]carbonyl}-6-fluoro-5-hydroxy-2-methyl-1H-indol-3-yl)acetic acid;
(6-fluoro-5-hydroxy-2-methyl-1-{[5-(trifluoromethyl)thien-3-yl]carbonyl}-1H-indol-3-yl)acetic acid;
(6-fluoro-5-hydroxy-2-methyl-1-{[5-(methylthio)thien-3-yl]carbonyl}-1H-indol-3-yl)acetic acid;
[1-({5-[(difluoromethyl)thio]thien-3-yl}carbonyl)-6-fluoro-5-hydroxy-2-methyl-1H-indol-3-yl]acetic acid;
[6-fluoro-5-hydroxy-2-methyl-1-({5-[(trifluoromethyl)thio]thien-3-yl}carbonyl)-1H-indol-3-yl]acetic acid;
[6-fluoro-5-hydroxy-2-methyl-1-({5-[(pentafluoroethyl)thio]thien-3-yl}carbonyl)-1H-indol-3-yl]acetic acid;
[6-fluoro-5-hydroxy-2-methyl-1-({5-[(1,1,2,2-tetrafluoroethyl)thio]thien-3-yl}carbonyl)-1H-indol-3-yl]acetic acid; and
{1-[(5-cyanothien-3-yl)carbonyl]-6-fluoro-5-hydroxy-2-methyl-1H-indol-3-yl}acetic acid.

[6-chloro-5-hydroxy-2-methyl-1-(thien-3-ylcarbonyl)-1H-indol-3-yl]acetic acid derivatives having the formula: 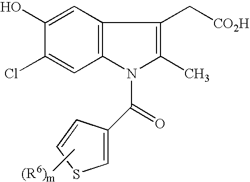
including:
[6-chloro-5-hydroxy-2-methyl-1-(thien-3-ylcarbonyl)-1H-indol-3-yl]acetic acid;
{6-chloro-1-[(5-fluorothien-3-yl)carbonyl -5-hydroxy-2-methyl-1H-indol-3-yl}acetic acid;
{1-[(5-chlorothien-3-yl)carbonyl]-6-chloro-5-hydroxy-2-methyl-1H-indol-3-yl}acetic acid;
{1-[(5-bromothien-3-yl)carbonyl]-6-chloro-5-hydroxy-2-methyl-1H-indol-3-yl}acetic acid;
{6-chloro-5-hydroxy-1-[(5-hydroxythien-3-yl)carbonyl]-2-methyl-1H-indol-3-yl}acetic acid;
{6-chloro-5-hydroxy-1-[(5-methoxythien-3-yl)carbonyl]-2-methyl-1H-indol-3-yl}acetic acid;
{1-[(5-ethoxythien-3-yl)carbonyl]-6-chloro-5-hydroxy-2-methyl-1H-indol-3-yl}acetic acid;
(1-{[5-(difluoromethoxy)thien-3-yl]carbonyl }-6-chloro-5-hydroxy-2-methyl-1H-indol-3-yl)acetic acid;
(6-chloro-5-hydroxy-2-methyl-1-{[5-(trifluoromethoxy)thien-3-yl]carbonyl}-H-indol-3-yl)acetic acid;
(6-chloro-5-hydroxy-2-methyl-1-{[5-(pentafluoroethoxy)thien-3-yl]carbonyl}-1H-indol-3-yl)acetic acid;
(6-chloro-5-hydroxy-2-methyl-1-{[5-(1,1,2,2-tetrafluoroethoxy)thien-3-yl]carbonyl}-1H-indol-3-yl)acetic acid;
{6-chloro-5-hydroxy-2-methyl-1-[(5-methylthien-3-yl)carbonyl]-1H-indol-3-yl}acetic acid;
(1-{[5-(difluoromethyl)thien-3-yl]carbonyl}-6-chloro-5-hydroxy-2-methyl-1H-indol-3-yl)acetic acid;
(6-chloro-5-hydroxy-2-methyl-1-{[5-(trifluoromethyl)thien-3-yl]carbonyl}-1H-indol-3-yl)acetic acid;
(6-chloro-5-hydroxy-2-methyl-1-{[5-(methylthio)thien-3-yl]carbonyl}-1H-indol-3-yl)acetic acid;
[1-({5-[(difluoromethyl)thio]thien-3-yl}carbonyl)-6-chloro-5-hydroxy-2-methyl-1H-indol-3-yl]acetic acid;
[6-chloro-5-hydroxy-2-methyl-1-({5-[(trifluoromethyl)thio]thien-3-yl}carbonyl)-1H-indol-3-yl]acetic acid;
[6-chloro-5-hydroxy-2-methyl-1-({5-[(pentafluoroethyl)thio]thien-3-yl}carbonyl)-1H-indol-3-yl]acetic acid;
[6-chloro-5-hydroxy-2-methyl-1-({5-[(1,1,2,2-tetrafluoroethyl)thio]thien-3-yl}carbonyl)-1H-indol-3-yl]acetic acid; and
{1-[(5-cyanothien-3-yl)carbonyl]-6-chloro-5-hydroxy-2-methyl-1H-indol-3-yl}acetic acid.

[6-chloro-5-hydroxy-2-methyl-1-(thien-2-ylcarbonyl)-1H-indol-3-yl]acetic acid derivatives having the formula: 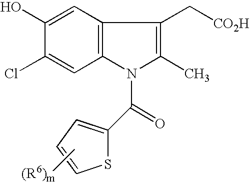
including:
[6-chloro-5-hydroxy-2-methyl-1-(thien-2-ylcarbonyl)-1H-indol-3-yl]acetic acid;
{6-chloro-1-[(5-fluorothien-2-yl)carbonyl]-5-hydroxy-2-methyl-1H-indol-3-yl}acetic acid;
{1-[(5-chlorothien-2-yl)carbonyl]-6-chloro-5-hydroxy-2-methyl-1H-indol-3-yl}acetic acid;
{1-[(5-bromothien-2-yl)carbonyl]-6-chloro-5-hydroxy-2-methyl-1H-indol-3-yl}acetic acid;
{6-chloro-5-hydroxy-1-[(5-hydroxythien-2-yl)carbonyl]-2-methyl-1H-indol-3-yl}acetic acid;
{6-chloro-5-hydroxy-1-[(5-methoxythien-2-yl)carbonyl]-2-methyl-1H-indol-3-yl}acetic acid;
{1-[(5-ethoxythien-2-yl)carbonyl]-6-chloro-5-hydroxy-2-methyl-1H-indol-3-yl}acetic acid;
(1-{[5-(difluoromethoxy)thien-2-yl]carbonyl}-6-chloro-5-hydroxy-2-methyl-1H-indol-3-yl)acetic acid;
(6-chloro-5-hydroxy-2-methyl-1-{[5-(trifluoromethoxy)thien-2-yl]carbonyl}-H-indol-3-yl)acetic acid;
(6-chloro-5-hydroxy-2-methyl-1-{[5-(pentafluoroethoxy)thien-2-yl]carbonyl}-1H-indol-3-yl)acetic acid;
(6-chloro-5-hydroxy-2-methyl-1-{[5-(1,1,2,2-tetrafluoroethoxy)thien-2-yl]carbonyl}-1-H-indol-3-yl)acetic acid;
{6-chloro-5-hydroxy-2-methyl-1-[(5-methylthien-2-yl)carbonyl]-1H-indol-3-yl}acetic acid;
(1-{[5-(difluoromethyl)thien-2-yl]carbonyl}-6-chloro-5-hydroxy-2-methyl-1H-indol-3-yl)acetic acid;
(6-chloro-5-hydroxy-2-methyl-1-{[5-(trifluoromethyl)thien-2-yl]carbonyl }-1H-indol-yl)acetic acid;
(6-chloro-5-hydroxy-2-methyl-1-{[5-(methylthio)thien-2-yl]carbonyl}-1H-indol-3-yl)acetic acid;
[1-({5-[(difluoromethyl)thio]thien-2-yl}carbonyl)-6-chloro-5-hydroxy-2-methyl-1H-indol-3-yl]acetic acid;
[6-chloro-5-hydroxy-2-methyl-1-({5-[(trifluoromethyl)thio]thien-2-yl}carbonyl)-1H-indol-3-yl]acetic acid;
[6-chloro-5-hydroxy-2-methyl-1-({5-[(pentafluoroethyl)thio]thien-2-yl}carbonyl)-1H-indol-3-yl]acetic acid;
[6-chloro-5-hydroxy-2-methyl-1-({5-[(1,1,2,2-tetrafluoroethyl)thio]thien-2-yl}carbonyl)-1H-indol-3-yl]acetic acid; and
{1-[(5-cyanothien-2-yl)carbonyl]-6-chloro-5-hydroxy-2-methyl-1H-indol-3-yl}acetic acid.

[6-fluoro-5-methoxy-2-methyl-1-(thien-3-ylcarbonyl)-1H-indol-3-yl]acetic acid derivatives having the formula: 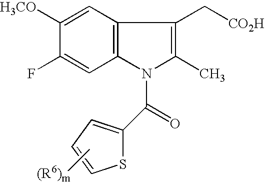
including:
[6-fluoro-5-methoxy-2-methyl-1-(thien-2-ylcarbonyl)-1H-indol-3-yl]acetic acid;
{6-fluoro-1-[(5-fluorothien-2-yl)carbonyl]-5-methoxy-2-methyl-1H-indol-3-yl}acetic acid;
{1-[(5-chlorothien-2-yl)carbonyl]-6-fluoro-5-methoxy-2-methyl-1H-indol-3-yl}acetic acid;
{1-[(5-bromothien-2-yl)carbonyl]-6-fluoro-5-methoxy-2-methyl-1H-indol-3-yl}acetic acid;
{6-fluoro-5-methoxy-1-[(5-hydroxythien-2-yl)carbonyl]-2-methyl-1H-indol-3-yl}acetic acid;
{6-fluoro-5-methoxy-1-[(5-methoxythien-2-yl)carbonyl]-2-methyl-1H-indol-3-yl}acetic acid;
{1-[(5-ethoxythien-2-yl)carbonyl]-6-fluoro-5-methoxy-2-methyl-1H-indol-3-yl}acetic acid;
(1-{[5-(difluoromethoxy)thien-2-yl]carbonyl}-6-fluoro-5-methoxy-2-methyl-1H-indol-3-yl)acetic acid;
(6-fluoro-5-methoxy-2-methyl-1-{[5-(trifluoromethoxy)thien-2-yl]carbonyl}-1H-indol-3-yl)acetic acid;
(6-fluoro-5-methoxy-2-methyl-1-{[5-(pentafluoroethoxy)thien-2-yl]carbonyl}-H-indol-3-yl)acetic acid;
(6-fluoro-5-methoxy-2-methyl-1-{[5-( 1,1,2,2-tetrafluoroethoxy)thien-2-yl]carbonyl}-1H-indol-3-yl)acetic acid;
{6-fluoro-5-methoxy-2-methyl-1-[(5-methylthien-2-yl)carbonyl]-1H-indol-3-yl}acetic acid;
(1-{[5-(difluoromethyl)thien-2-yl]carbonyl}-6-fluoro-5-methoxy-2-methyl-1H-indol-3-yl)acetic acid;
(6-fluoro-5-methoxy-2-methyl-1-{[5-(trifluoromethyl)thien-2-yl]carbonyl}-1H-indol-3-yl)acetic acid;
(6-fluoro-5-methoxy-2-methyl-1-{[5-(methylthio)thien-2-yl]carbonyl}-1H-indol-3-yl)acetic acid;
[1-({5-[(difluoromethyl)thio]thien-2-yl}carbonyl)-6-fluoro-5-methoxy-2-methyl-1H-indol-3-yl]acetic acid;
[6-fluoro-5-methoxy-2-methyl-1-({5-[(trifluoromethyl)thio]thien-2-yl}carbonyl)-1H-indol-3-yl]acetic acid;
[6-fluoro-5-methoxy-2-methyl-1-({5-[(pentafluoroethyl)thio]thien-2-yl}carbonyl)-1H-indol-3-yl]acetic acid;
[6-fluoro-5-methoxy-2-methyl-1-({5-[(1,1,2,2-tetrafluoroethyl)thio]thien-2-yl}carbonyl)-1H-indol-3-yl]acetic acid; and
{1-[(5-cyanothien-2-yl)carbonyl]-6-fluoro-5-methoxy-2-methyl-1H-indol-3-yl}acetic acid.

[6-chloro-5-methoxy-2-methyl-1-(thien-2-ylcarbonyl)-1H-indol-3-yl]acetic acid derivatives having the formula: 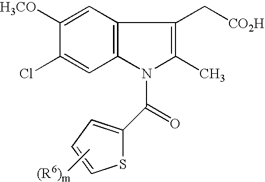
including:
[6-chloro-5-methoxy-2-methyl-1-(thien-2-ylcarbonyl)-1H-indol-3-yl]acetic acid;
{6-chloro-1-[(5-fluorothien-2-yl)carbonyl]-5-methoxy-2-methyl-1H-indol-3-yl}acetic acid;
{1-[(5-chlorothien-2-yl)carbonyl]-6-chloro-5-methoxy-2-methyl-1H-indol-3-yl}acetic acid;
{1-[(5-bromothien-2-yl)carbonyl]-6-chloro-5-methoxy-2-methyl-1H-indol-3-yl}acetic acid;
{6-chloro-5-methoxy-1-[(5-hydroxythien-2-yl)carbonyl]-2-methyl-1H-indol-3-yl}acetic acid;
{6-chloro-5-methoxy-1-[(5-methoxythien-2-yl)carbonyl]-2-methyl-1H-indol-3-yl}acetic acid;
{1-[(5-ethoxythien-2-yl)carbonyl]-6-chloro-5-methoxy-2-methyl-1H-indol-3-yl}acetic acid;
(1-{[5-(difluoromethoxy)thien-2-yl]carbonyl}-6-chloro-5-methoxy-2-methyl-1H-indol-3-yl)acetic acid;
(6-chloro-5-methoxy-2-methyl-1-{[5-(trifluoromethoxy)thien-2-yl]carbonyl}-1H-indol-3-yl)acetic acid;
(6-chloro-5-methoxy-2-methyl-1-{[5-(pentafluoroethoxy)thien-2-yl]carbonyl}-1H-indol-3-yl)acetic acid;
(6-chloro-5-methoxy-2-methyl-1-{[5-(1,1,2,2-tetrafluoroethoxy)thien-2-yl]carbonyl}-1H-indol-3-yl)acetic acid;
{6-chloro-5-methoxy-2-methyl-1-[(5-methylthien-2-yl)carbonyl]-1H-indol-3-yl}acetic acid;
(1-{[5-(difluoromethyl)thien-2-yl]carbonyl}-6-chloro-5-methoxy-2-methyl-1H-indol-3-yl)acetic acid;
(6-chloro-5-methoxy-2-methyl-1-{[5-(trifluoromethyl)thien-2-yl]carbonyl)}-H-indol-3-yl)acetic acid;
(6-chloro-5-methoxy-2-methyl-1-{[5-(methylthio)thien-2-yl]carbonyl}-1H-indol-3-yl)acetic acid;
[1-({5-[(difluoromethyl)thio]thien-2-yl}carbonyl)-6-chloro-5-methoxy-2-methyl-1H-indol-3-yl]acetic acid;
[6-chloro-5-methoxy-2-methyl-1-({5-[(trifluoromethyl)thio]thien-2-yl}carbonyl)-1H-indol-3-yl]acetic acid;
[6-chloro-5-methoxy-2-methyl-1-({5-[(pentafluoroethyl)thio]thien-2-yl}carbonyl)-1H-indol-3-yl]acetic acid;
[6-chloro-5-methoxy-2-methyl-1-({5-[(1,1,2,2-tetrafluoroethyl)thio]thien-2-yl}carbonyl)-1H-indol-3-yl]acetic acid; and
{1-[(5-cyanothien-2-yl)carbonyl]-6-chloro-5-methoxy-2-methyl-1H-indol-3-yl}acetic acid.

[6-fluoro-5-methoxy-2-methyl-1-(thien-3-ylcarbonyl)-1H-indol-3-yl]acetic acid derivatives having the formula: 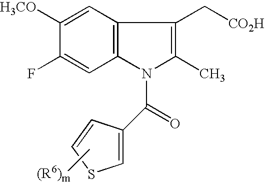
including:
[6-fluoro-5-methoxy-2-methyl-1-(thien-3-ylcarbonyl)-1H-indol-3-yl]acetic acid;
{6-fluoro-1-[(5-fluorothien-3-yl)carbonyl]-5-methoxy-2-methyl-1H-indol-3-yl}acetic acid;
{1-[(5-chlorothien-3-yl)carbonyl]-6-fluoro-5-methoxy-2-methyl-1H-indol-3-yl}acetic acid;
{1-[(5-bromothien-3-yl)carbonyl]-6-fluoro-5-methoxy-2-methyl-1H-indol-3-yl}acetic acid;
{6-fluoro-5-methoxy-1-[(5-hydroxythien-3-yl)carbonyl]-2-methyl-1H-indol-3-yl}acetic acid;
{6-fluoro-5-methoxy-1-[(5-methoxythien-3-yl)carbonyl]-2-methyl-1H-indol-3-yl}acetic acid;
{1-[(5-ethoxythien-3-yl)carbonyl]-6-fluoro-5-methoxy-2-methyl-1H-indol-3-yl}acetic acid;
(1-{[5-(difluoromethoxy)thien-3-yl]carbonyl}-6-fluoro-5-methoxy-2-methyl-1H-indol-3-yl)acetic acid;
(6-fluoro-5-methoxy-2-methyl-1-{[5-(trifluoromethoxy)thien-3-yl]carbonyl}-1H-indol-3-yl)acetic acid;
(6-fluoro-5-methoxy-2-methyl-1-{[5-(pentafluoroethoxy)thien-3-yl]carbonyl)}-H-indol-3-yl)acetic acid;
(6-fluoro-5-methoxy-2-methyl-1-{[5-( 1,1,2,2-tetrafluoroethoxy)thien-3-yl]carbonyl}-1H-indol-3-yl)acetic acid;
{6-fluoro-5-methoxy-2-methyl-1-[(5-methylthien-3-yl)carbonyl]-1H-indol-3-yl}acetic acid;
(1-{[5-(difluoromethyl)thien-3-yl]carbonyl}-6-fluoro-5-methoxy-2-methyl-1H-indol-3-yl)acetic acid;
(6-fluoro-5-methoxy-2-methyl-1-{[5-(trifluoromethyl)thien-3-yl]carbonyl}-H-indol-3-yl)acetic acid;
(6-fluoro-5-methoxy-2-methyl-1-{[5-(methylthio)thien-3-yl]carbonyl}-1H-indol-3-yl)acetic acid;
[1-({5-[(difluoromethyl)thio]thien-3-yl}carbonyl)-6-fluoro-5-methoxy-2-methyl-1H-indol-3-yl]acetic acid;
[6-fluoro-5-methoxy-2-methyl-1-({5-[(trifluoromethyl)thio]thien-3-yl}carbonyl)-1H-indol-3-yl]acetic acid;
[6-fluoro-5-methoxy-2-methyl-1-({5-[(pentafluoroethyl)thio]thien-3-yl}carbonyl)-1H-indol-3-yl]acetic acid;
[6-fluoro-5-methoxy-2-methyl-1-({5-[(1,1,2,2-tetrafluoroethyl)thio]thien-3-yl}carbonyl)-1H-indol-3-yl]acetic acid; and
{1-[(5-cyanothien-3-yl)carbonyl]-6-fluoro-5-methoxy-2-methyl-1H-indol-3-yl}acetic acid.

[6-chloro-5-methoxy-2-methyl-1-(thien-3-ylcarbonyl)-1H-indol-3-yl]acetic acid derivatives having the formula: 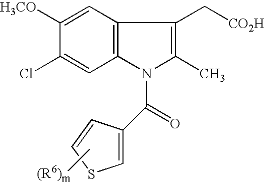
including:
[6-chloro-5-methoxy-2-methyl-1-(thien-3-ylcarbonyl)-1H-indol-3-yl]acetic acid;
{6-chloro-1-[(5-fluorothien-3-yl)carbonyl]-5-methoxy-2-methyl-1H-indol-3-yl}acetic acid;
{1-[(5-chlorothien-3-yl)carbonyl]-6-chloro-5-methoxy-2-methyl-1H-indol-3-yl}acetic acid;
{1-[(5-bromothien-3-yl)carbonyl]-6-chloro-5-methoxy-2-methyl-1H-indol-3-yl}acetic acid;
{6-chloro-5-methoxy-1-[(5-hydroxythien-3-yl)carbonyl]-2-methyl-1H-indol-3-yl}acetic acid;
{6-chloro-5-methoxy-1-[(5-methoxythien-3-yl)carbonyl]-2-methyl-1H-indol-3-yl}acetic acid;
{1-[(5-ethoxythien-3-yl)carbonyl]-6-chloro-5-methoxy-2-methyl-1H-indol-3-yl}acetic acid;
(1-{[5-(difluoromethoxy)thien-3-yl]carbonyl}-6-chloro-5-methoxy-2-methyl-1H-indol-3-yl)acetic acid;
(6-chloro-5-methoxy-2-methyl-1-{[5-(trifluoromethoxy)thien-3-yl]carbonyl}-1H-indol-3-yl)acetic acid;
(6-chloro-5-methoxy-2-methyl-1-{[5-(pentafluoroethoxy)thien-3-yl]carbonyl}-H-indol-3-yl)acetic acid;
(6-chloro-5-methoxy-2-methyl-1-{[5-( 1,1,2,2-tetrafluoroethoxy)thien-3-yl]carbonyl)}1H-indol-3-yl)acetic acid;
{6-chloro-5-methoxy-2-methyl-1-[(5-methylthien-3-yl)carbonyl]-1H-indol-3-yl}acetic acid;
(1-{[5-(difluoromethyl)thien-3-yl]carbonyl}-6-chloro-5-methoxy-2-methyl-1H-indol-3-yl)acetic acid;
(6-chloro-5-methoxy-2-methyl-1-{[5-(trifluoromethyl)thien-3-yl]carbonyl}-H-indol-3-yl)acetic acid;
(6-chloro-5-methoxy-2-methyl-1-{[5-(methylthio)thien-3-yl]carbonyl}-1H-indol-3-yl)acetic acid;
[1-({5-[(difluoromethyl)thio]thien-3-yl}carbonyl)-6-chloro-5-methoxy-2-methyl-1H-indol-3-yl]acetic acid;
[6-chloro-5-methoxy-2-methyl-1-({5-[(trifluoromethyl)thio]thien-3-yl}carbonyl)-1H-indol-3-yl]acetic acid;
[6-chloro-5-methoxy-2-methyl-1-({5-[(pentafluoroethyl)thio]thien-3-yl}carbonyl)-1H-indol-3-yl]acetic acid;
[6-chloro-5-methoxy-2-methyl-1-({5-[(1,1,2,2-tetrafluoroethyl)thio]thien-3-yl}carbonyl)-1H-indol-3-yl]acetic acid; and
{1-[(5-cyanothien-3-yl)carbonyl]-6-chloro-5-methoxy-2-methyl-1H-indol-3-yl}acetic acid.

[4-fluoro-5-hydroxy-2-methyl-1-(thien-2-ylcarbonyl)-1H-indol-3-yl]acetic acid derivatives having the formula: 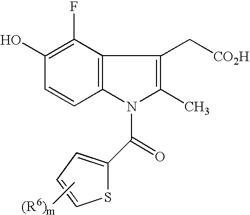
including:
[4-fluoro-5-hydroxy-2-methyl-1-(thien-2-ylcarbonyl)-1H-indol-3-yl]acetic acid;
{4-fluoro-1-[(5-fluorothien-2-yl)carbonyl]-5-hydroxy-2-methyl-1H-indol-3-yl}acetic acid;
{1-[(5-chlorothien-2-yl)carbonyl]-4-fluoro-5-hydroxy-2-methyl-1H-indol-3-yl}acetic acid;
{1-[(5-bromothien-2-yl)carbonyl]-4-fluoro-5-hydroxy-2-methyl-1H-indol-3-yl}acetic acid;
{4-fluoro-5-hydroxy-1-[(5-hydroxythien-2-yl)carbonyl]-2-methyl-1H-indol-3-yl}acetic acid;
{4-fluoro-5-hydroxy-1-[(5-methoxythien-2-yl)carbonyl]-2-methyl-1H-indol-3-yl}acetic acid;
{1-[(5-ethoxythien-2-yl)carbonyl]-4-fluoro-5-hydroxy-2-methyl-1H-indol-3-yl}acetic acid;
(1-{[5-(difluoromethoxy)thien-2-yl]carbonyl}-4-fluoro-5-hydroxy-2-methyl-1H-indol-3-yl)acetic acid;
(4-fluoro-5-hydroxy-2-methyl-1-{[5-(trifluoromethoxy)thien-2-yl]carbonyl}-H-indol-3-yl)acetic acid;
(4-fluoro-5-hydroxy-2-methyl-1-{[5-(pentafluoroethoxy)thien-2-yl]carbonyl}-1H-indol-3-yl)acetic acid;
(4-fluoro-5-hydroxy-2-methyl-1-{[5-(1,1,2,2-tetrafluoroethoxy)thien-2-yl]carbonyl}-indol-3-yl)acetic acid;
{4-fluoro-5-hydroxy-2-methyl-1-[(5-methylthien-2-yl)carbonyl]-1H-indol-3-yl}acetic acid;
(1-{[5-(difluoromethyl)thien-2-yl]carbonyl}-4-fluoro-5-hydroxy-2-methyl-1H-indol-3-yl)acetic acid;
(4-fluoro-5-hydroxy-2-methyl-1-{[5-(trifluoromethyl)thien-2-yl]carbonyl}-1H-indol-3-yl)acetic acid;
(4-fluoro-5-hydroxy-2-methyl-1-{[5-(methylthio)thien-2-yl]carbonyl}-1H-indol-3-yl)acetic acid;
[1-({5-[(difluoromethyl)thio]thien-2-yl}carbonyl)-4-fluoro-5-hydroxy-2-methyl-1H-indol-3-yl]acetic acid;
[4-fluoro-5-hydroxy-2-methyl-1-({5-[(trifluoromethyl)thio]thien-2-yl}carbonyl)-1H-indol-3-yl]acetic acid;
[4-fluoro-5-hydroxy-2-methyl-1-({5-[(pentafluoroethyl)thio]thien-2-yl}carbonyl)-1H-indol-3-yl]acetic acid;
[4-fluoro-5-hydroxy-2-methyl-1-({5-[(1,1,2,2-tetrafluoroethyl)thio]thien-2-yl}carbonyl)-1H-indol-3-yl]acetic acid; and
{1-[(5-cyanothien-2-yl)carbonyl]-4-fluoro-5-hydroxy-2-methyl-1H-indol-3-yl}acetic acid

[4-fluoro-5-hydroxy-2-methyl-1-(thien-3-ylcarbonyl)-1H-indol-3-yl]acetic acid derivatives having the formula: 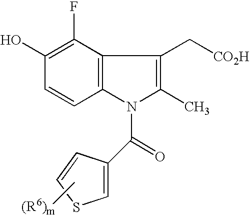
including:
[4-fluoro-5-hydroxy-2-methyl-1-(thien-3-ylcarbonyl)-1H-indol-3-yl]acetic acid;
{4-fluoro-1-[(5-fluorothien-3-yl)carbonyl]-5-hydroxy-2-methyl-1H-indol-3-yl}acetic acid;
{1-[(5-chlorothien-3-yl)carbonyl]-4-fluoro-5-hydroxy-2-methyl-1H-indol-3-yl}acetic acid;
{1-[(5-bromothien-3-yl)carbonyl]-4-fluoro-5-hydroxy-2-methyl-1H-indol-3-yl}acetic acid;
{4-fluoro-5-hydroxy-1-[(5-hydroxythien-3-yl)carbonyl]-2-methyl-1H-indol-3-yl}acetic acid;
{4-fluoro-5-hydroxy-1-[(5-methoxythien-3-yl)carbonyl]-2-methyl-1H-indol-3-yl}acetic acid;
{1-[(5-ethoxythien-3-yl)carbonyl]-4-fluoro-5-hydroxy-2-methyl-1H-indol-3-yl}acetic acid;
(1-{[5-(difluoromethoxy)thien-3-yl]carbonyl}-4-fluoro-5-hydroxy-2-methyl-1H-indol-3-yl)acetic acid;
(4-fluoro-5-hydroxy-2-methyl-1-{[5-(trifluoromethoxy)thien-3-yl]carbonyl}-1H-indol-3-yl)acetic acid;
(4-fluoro-5-hydroxy-2-methyl-1-{[5-(pentafluoroethoxy)thien-3-yl]carbonyl}-1H-indol-3-yl)acetic acid;
(4-fluoro-5-hydroxy-2-methyl-1-{[5-(1,1,2,2-tetrafluoroethoxy)thien-3-yl]carbonyl}-1H-indol-3-yl)acetic acid;
{4-fluoro-5-hydroxy-2-methyl-1-[(5-methylthien-3-yl)carbonyl]-1H-indol-3-yl }acetic acid;
(1-{[5-(difluoromethyl)thien-3-yl]carbonyl}-4-fluoro-5-hydroxy-2-methyl-1H-indol-3-yl)acetic acid;
(4-fluoro-5-hydroxy-2-methyl-1-{[5-(trifluoromethyl)thien-3-yl]carbonyl}-1H-indol-3-yl)acetic acid;
(4-fluoro-5-hydroxy-2-methyl-1-{[5-(methylthio)thien-3-yl]carbonyl}-1H-indol-3-yl)acetic acid;
[1-({5-[(difluoromethyl)thio]thien-3-yl}carbonyl)-4-fluoro-5-hydroxy-2-methyl-1H-indol-3-yl]acetic acid;
[4-fluoro-5-hydroxy-2-methyl-1-({5-[(trifluoromethyl)thio]thien-3-yl}carbonyl)-1H-indol-3-yl]acetic acid;
[4-fluoro-5-hydroxy-2-methyl-1-({5-[(pentafluoroethyl)thio]thien-3-yl}carbonyl)-1H-indol-3-yl]acetic acid;
[4-fluoro-5-hydroxy-2-methyl-1-({5-[(1,1,2,2-tetrafluoroethyl)thio]thien-3-yl}carbonyl)-1H-indol-3-yl]acetic acid; and
{1-[(5-cyanothien-3-yl)carbonyl]-4-fluoro-5-hydroxy-2-methyl-1H-indol-3-yl}acetic acid.

[4-chloro-5-hydroxy-2-methyl-1-(thien-3-ylcarbonyl)-1H-indol-3-yl]acetic acid derivatives having the formula: 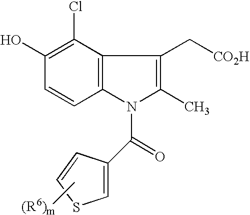
including:
[4-chloro-5-hydroxy-2-methyl-1-(thien-3-ylcarbonyl)-1H-indol-3-yl]acetic acid;
{4-chloro-1-[(5-fluorothien-3-yl)carbonyl]-5-hydroxy-2-methyl-1H-indol-3-yl}acetic acid;
{1-[(5-chlorothien-3-yl)carbonyl]-4-chloro-5-hydroxy-2-methyl-1H-indol-3-yl}acetic acid;
{1-[(5-bromothien-3-yl)carbonyl]-4-chloro-5-hydroxy-2-methyl-1H-indol-3-yl}acetic acid;
{4-chloro-5-hydroxy-1-[(5-hydroxythien-3-yl)carbonyl]-2-methyl-1H-indol-3-yl}acetic acid;
{4-chloro-5-hydroxy-1-[(5-methoxythien-3-yl)carbonyl]-2-methyl-1H-indol-3-yl}acetic acid;
{1-[(5-ethoxythien-3-yl)carbonyl]-4-chloro-5-hydroxy-2-methyl-1H-indol-3-yl}acetic acid;
(1-{[5-(difluoromethoxy)thien-3-yl]carbonyl}-4-chloro-5-hydroxy-2-methyl-1H-indol-3-yl)acetic acid;
(4-chloro-5-hydroxy-2-methyl-1-{[5-(trifluoromethoxy)thien-3-yl]carbonyl}-1H-indol-3-yl)acetic acid;
(4-chloro-5-hydroxy-2-methyl-1-{[5-(pentafluoroethoxy)thien-3.-yl]carbonyl}-1H-indol-3-yl)acetic acid;
(4-chloro-5-hydroxy-2-methyl-1-{[5-(1,1,2,2-tetrafluoroethoxy)thien-3-yl]carbonyl}-1H-indol-3-yl)acetic acid;
{4-chloro-5-hydroxy-2-methyl-1-[(5-methylthien-3-yl)carbonyl]-1H-indol-3-yl}acetic acid;
(1-{[5-(difluoromethyl)thien-3-yl]carbonyl}-4-chloro-5-hydroxy-2-methyl-1H-indol-3-yl)acetic acid;
(4-chloro-5-hydroxy-2-methyl-1-{[5-(trifluoromethyl)thien-3-yl]carbonyl}-1H-indol-3-yl)acetic acid;
(4-chloro-5-hydroxy-2-methyl-1-{[5-(methylthio)thien-3-yl]carbonyl}-1H-indol-3-yl)acetic acid;
[1-({5-[(difluoromethyl)thio]thien-3-yl}carbonyl)-4-chloro-5-hydroxy-2-methyl-1H-indol-3-yl]acetic acid;
[4-chloro-5-hydroxy-2-methyl-1-({5-[(trifluoromethyl)thio]thien-3-yl}carbonyl)-1H-indol-3-yl]acetic acid;
[4-chloro-5-hydroxy-2-methyl-1-({5-[(pentafluoroethyl)thio]thien-3-yl}carbonyl)-1H-indol-3-yl]acetic acid;
[4-chloro-5-hydroxy-2-methyl-1-({5-[(1,1,2,2-tetrafluoroethyl)thio]thien-3-yl}carbonyl)-1H-indol-3-yl]acetic acid; and
{1-[(5-cyanothien-3-yl)carbonyl]-4-chloro-5-hydroxy-2-methyl-1H-indol-3-yl}acetic acid.

[4-chloro-5-hydroxy-2-methyl-1-(thien-2-ylcarbonyl)-1H-indol-3-yl]acetic acid derivatives having the formula: 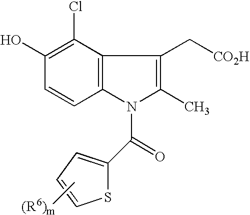
including:
[4-chloro-5-hydroxy-2-methyl-1-(thien-2-ylcarbonyl)-1H-indol-3-yl]acetic acid;
{4-chloro-1-[(5-fluorothien-2-yl)carbonyl]-5-hydroxy-2-methyl-1H-indol-3-yl}acetic acid;
{1-[(5-chlorothien-2-yl)carbonyl]-4-chloro-5-hydroxy-2-methyl-1H-indol-3-yl}acetic acid;
{1-[(5-bromothien-2-yl)carbonyl]-4-chloro-5-hydroxy-2-methyl-1H-indol-3-yl}acetic acid;
{4-chloro-5-hydroxy-1-[(5-hydroxythien-2-yl)carbonyl]-2-methyl-1H-indol-3-yl}acetic acid;
{4-chloro-5-hydroxy-1-[(5-methoxythien-2-yl)carbonyl]-2-methyl-1H-indol-3-yl}acetic acid;
{1-[(5-ethoxythien-2-yl)carbonyl]-4-chloro-5-hydroxy-2-methyl-1H-indol-3-yl}acetic acid;
(1-{[5-(difluoromethoxy)thien-2-yl]carbonyl}-4-chloro-5-hydroxy-2-methyl-1H-indol-3-yl)acetic acid;
(4-chloro-5-hydroxy-2-methyl-1-{[5-(trifluoromethoxy)thien-2-yl]carbonyl}-1H-indol-3-yl)acetic acid;
(4-chloro-5-hydroxy-2-methyl-1-{[5-(pentafluoroethoxy)thien-2-yl]carbonyl}-1H-indol-3-yl)acetic acid;
(4-chloro-5-hydroxy-2-methyl-1-{[5-(1,1,2,2-tetrafluoroethoxy)thien-2-yl]carbonyl}-1H-indol-3-yl)acetic acid;
{4-chloro-5-hydroxy-2-methyl-1-[(5-methylthien-2-yl)carbonyl]-1H-indol-3-yl}acetic acid;
(1-{[5-(difluoromethyl)thien-2-yl]carbonyl}-4-chloro-5-hydroxy-2-methyl-1H-indol-3-yl)acetic acid;
(4-chloro-5-hydroxy-2-methyl-1-{[5-(trifluoromethyl)thien-2-yl]carbonyl}-H-indol-3-yl)acetic acid;
(4-chloro-5-hydroxy-2-methyl-1-{[5-(methylthio)thien-2-yl]carbonyl)}-H-indol-3-yl)acetic acid;
[1-({5-[(difluoromethyl)thio]thien-2-yl}carbonyl)-4-chloro-5-hydroxy-2-methyl-1H-indol-3-yl]acetic acid;
[4-chloro-5-hydroxy-2-methyl-1-({5-[(trifluoromethyl)thio]thien-2-yl}carbonyl)-1H-indol-3-yl]acetic acid;
[4-chloro-5-hydroxy-2-methyl-1-({5-[(pentafluoroethyl)thio]thien-2-yl}carbonyl)-1H-indol-3-yl]acetic acid;
[4-chloro-5-hydroxy-2-methyl-1-({5-[(1,1,2,2-tetrafluoroethyl)thio]thien-2-yl}carbonyl)-1H-indol-3-yl]acetic acid; and
{1-[(5-cyanothien-2-yl)carbonyl]-4-chloro-5-hydroxy-2-methyl-1H-indol-3-yl}acetic acid.

[4-fluoro-5-methoxy-2-methyl-1-(thien-3-ylcarbonyl)-1H-indol-3-yl]acetic acid derivatives having the formula: 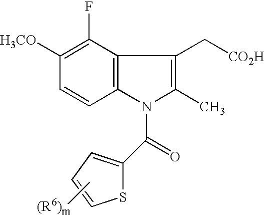
including:
[4-fluoro-5-methoxy-2-methyl-1-(thien-2-ylcarbonyl)-1H-indol-3-yl]acetic acid;
{4-fluoro-1-[(5-fluorothien-2-yl)carbonyl]-5-methoxy-2-methyl-1H-indol-3-yl}acetic acid;
{1-[(5-chlorothien-2-yl)carbonyl]-4-fluoro-5-methoxy-2-methyl-1H-indol-3-yl}acetic acid;
{1-[(5-bromothien-2-yl)carbonyl]-4-fluoro-5-methoxy-2-methyl-1H-indol-3-yl}acetic acid;
{4-fluoro-5-methoxy-1-[(5-hydroxythien-2-yl)carbonyl]-2-methyl-1H-indol-3-yl}acetic acid;
{4-fluoro-5-methoxy-1-[(5-methoxythien-2-yl)carbonyl]-2-methyl-1H-indol-3-yl}acetic acid;
{1-[(5-ethoxythien-2-yl)carbonyl]-4-fluoro-5-methoxy-2-methyl-1H-indol-3-yl}acetic acid;
(1-{[5-(difluoromethoxy)thien-2-yl]carbonyl}-4-fluoro-5-methoxy-2-methyl-1H-indol-3-yl)acetic acid;
(4-fluoro-5-methoxy-2-methyl-1-{[5-(trifluoromethoxy)thien-2-yl]carbonyl}-1H-indol-3-yl)acetic acid;
(4-fluoro-5-methoxy-2-methyl-1-{[5-(pentafluoroethoxy)thien-2-yl]carbonyl}-1H-indol-3-yl)acetic acid;
(4-fluoro-5-methoxy-2-methyl-1-{[5-(1,1,2,2-tetrafluoroethoxy)thien-2-yl]carbonyl}-1H-indol-3-yl)acetic acid;
{4-fluoro-5-methoxy-2-methyl-1-[(5-methylthien-2-yl)carbonyl]-1H-indol-3-yl}acetic acid;
(1-{[5-(difluoromethyl)thien-2-yl]carbonyl}-4-fluoro-5-methoxy-2-methyl-1H-indol-3-yl)acetic acid;
(4-fluoro-5-methoxy-2-methyl-1-{[5-(trifluoromethyl)thien-2-yl]carbonyl}-1H-indol-3-yl)acetic acid;
(4-fluoro-5-methoxy-2-methyl-1-{[5-(methylthio)thien-2-yl]carbonyl}-1H-indol-3-yl)acetic acid;
[1-({5-[(difluoromethyl)thio]thien-2-yl}carbonyl)-4-fluoro-5-methoxy-2-methyl-1H-indol-3-yl]acetic acid;
[4-fluoro-5-methoxy-2-methyl-1-({5-[(trifluoromethyl)thio]thien-2-yl}carbonyl)-1H-indol-3-yl]acetic acid;
[4-fluoro-5-methoxy-2-methyl-1-({5-[(pentafluoroethyl)thio]thien-2-yl}carbonyl)-1H-indol-3-yl]acetic acid;
[4-fluoro-5-methoxy-2-methyl-1-({5-[(1,1,2,2-tetrafluoroethyl)thio]thien-2-yl}carbonyl)-1H-indol-3-yl]acetic acid; and
{1-[(5-cyanothien-2-yl)carbonyl]-4-fluoro-5-methoxy-2-methyl-1H-indol-3-yl}acetic acid.

[4-chloro-5-methoxy-2-methyl-1-(thien-2-ylcarbonyl)-1H-indol-3-yl]acetic acid derivatives having the formula: 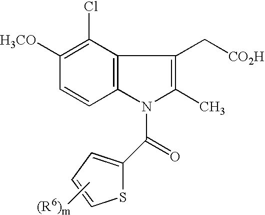
including:
[4-chloro-5-methoxy-2-methyl-1-(thien-2-ylcarbonyl)-1H-indol-3-yl]acetic acid;
{4-chloro-1-[(5-fluorothien-2-yl)carbonyl]-5-methoxy-2-methyl-1H-indol-3-yl}acetic acid;
{1-[(5-chlorothien-2-yl)carbonyl]-4-chloro-5-methoxy-2-methyl-1H-indol-3-yl}acetic acid;
{1-[(5-bromothien-2-yl)carbonyl]-4-chloro-5-methoxy-2-methyl-1H-indol-3-yl}acetic acid;
{4-chloro-5-methoxy-1-[(5-hydroxythien-2-yl)carbonyl]-2-methyl-1H-indol-3-yl}acetic acid;
{4-chloro-5-methoxy-1-[(5-methoxythien-2-yl)carbonyl]-2-methyl-1H-indol-3-yl}acetic acid;
{1-[(5-ethoxythien-2-yl)carbonyl]-4-chloro-5-methoxy-2-methyl-1H-indol-3-yl}acetic acid;
(1-{[5-(difluoromethoxy)thien-2-yl]carbonyl}-4-chloro-5-methoxy-2-methyl-1H-indol-3-yl)acetic acid;
(4-chloro-5-methoxy-2-methyl-1-{[5-(trifluoromethoxy)thien-2-yl]carbonyl)}-H-indol-3-yl)acetic acid;
(4-chloro-5-methoxy-2-methyl-1-{[5-(pentafluoroethoxy)thien-2-yl]carbonyl}-1H-indol-3-yl)acetic acid;
(4-chloro-5-methoxy-2-methyl-1-{[5-(1,1,2,2-tetrafluoroethoxy)thien-2-yl]carbonyl}-indol-3-yl)acetic acid;
{4-chloro-5-methoxy-2-methyl-1-[(5-methylthien-2-yl)carbonyl]-1H-indol-3-yl}acetic acid;
(1-{[5-(difluoromethyl)thien-2-yl]carbonyl}-4-chloro-5-methoxy-2-methyl-1H-indol-3-yl)acetic acid;
(4-chloro-5-methoxy-2-methyl-1-{[5-(trifluoromethyl)thien-2-yl]carbonyl}-1H-indol-3-yl)acetic acid;
(4-chloro-5-methoxy-2-methyl-1-{[5-(methylthio)thien-2-yl]carbonyl}-1H-indol-3-yl)acetic acid;
[1-({5-[(difluoromethyl)thio]thien-2-yl}carbonyl)-4-chloro-5-methoxy-2-methyl-1H-indol-3-yl]acetic acid;
[4-chloro-5-methoxy-2-methyl-1-({5-[(trifluoromethyl)thio]thien-2-yl}carbonyl)-1H-indol-3-yl]acetic acid;
[4-chloro-5-methoxy-2-methyl-1-({5-[(pentafluoroethyl)thio]thien-2-yl}carbonyl)-1H-indol-3-yl]acetic acid;
[4-chloro-5-methoxy-2-methyl-1-({5-[(1,1,2,2-tetrafluoroethyl)thio]thien-2-yl}carbonyl)-1H-indol-3-yl]acetic acid; and
{1-[(5-cyanothien-2-yl)carbonyl]-4-chloro-5-methoxy-2-methyl-1H-indol-3-yl}acetic acid.

[4-fluoro-5-methoxy-2-methyl-1-(thien-3-ylcarbonyl)-1H-indol-3-yl]acetic acid derivatives having the formula: 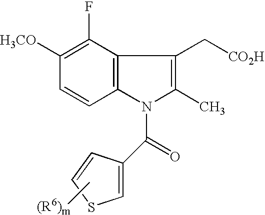
including:
[4-fluoro-5-methoxy-2-methyl-1-(thien-3-ylcarbonyl)-1H-indol-3-yl]acetic acid;
{4-fluoro-1-[(5-fluorothien-3-yl)carbonyl]-5-methoxy-2-methyl-1H-indol-3-yl}acetic acid;
{1-[(5-chlorothien-3-yl)carbonyl]-4-fluoro-5-methoxy-2-methyl-1H-indol-3-yl}acetic acid;
{1-[(5-bromothien-3-yl)carbonyl]-4-fluoro-5-methoxy-2-methyl-1H-indol-3-yl}acetic acid;
{4-fluoro-5-methoxy-1-[(5-hydroxythien-3-yl)carbonyl]-2-methyl-1H-indol-3-yl}acetic acid;
{4-fluoro-5-methoxy-1-[(5-methoxythien-3-yl)carbonyl]-2-methyl-1H-indol-3-yl}acetic acid;
{1-[(5-ethoxythien-3-yl)carbonyl]-4-fluoro-5-methoxy-2-methyl-1H-indol-3-yl}acetic acid;
(1-{[5-(difluoromethoxy)thien-3-yl]carbonyl}-4-fluoro-5-methoxy-2-methyl-1H-indol-3-yl)acetic acid;
(4-fluoro-5-methoxy-2-methyl-1-{[5-(trifluoromethoxy)thien-3-yl]carbonyl}-1H-indol-3-yl)acetic acid;
(4-fluoro-5-methoxy-2-methyl-1-{[5-(pentafluoroethoxy)thien-3-yl]carbonyl}-1H-indol-3-yl)acetic acid;
(4-fluoro-5-methoxy-2-methyl-1-{[5-(1,1,2,2-tetrafluoroethoxy)thien-3-yl]carbonyl}-indol-3-yl)acetic acid;
{4-fluoro-5-methoxy-2-methyl-1-[(5-methylthien-3-yl)carbonyl]-1H-indol-3-yl}acetic acid;
(1-{[5-(difluoromethyl)thien-3-yl]carbonyl}-4-fluoro-5-methoxy-2-methyl-1H-indol-3-yl)acetic acid;
(4-fluoro-5-methoxy-2-methyl-1-{[5-(trifluoromethyl)thien-3-yl]carbonyl}-1H-indol-3-yl)acetic acid;
(4-fluoro-5-methoxy-2-methyl-1-{[5-(methylthio)thien-3-yl]carbonyl}-1H-indol-3-yl)acetic acid;
[1-({5-[(difluoromethyl)thio]thien-3-yl}carbonyl)-4-fluoro-5-methoxy-2-methyl-1H-indol-3-yl]acetic acid;
[4-fluoro-5-methoxy-2-methyl-1-({5-[(trifluoromethyl)thio]thien-3-yl}carbonyl)-1H-indol-3-yl]acetic acid;
[4-fluoro-5-methoxy-2-methyl-1-({5-[(pentafluoroethyl)thio]thien-3-yl}carbonyl)-1H-indol-3-yl]acetic acid;
[4-fluoro-5-methoxy-2-methyl-1-({5-[(1,1,2,2-tetrafluoroethyl)thio]thien-3-yl}carbonyl)-1H-indol-3-yl]acetic acid; and
{1-[(5-cyanothien-3-yl)carbonyl]-4-fluoro-5-methoxy-2-methyl-1H-indol-3-yl}acetic acid.

[4-chloro-5-methoxy-2-methyl-1-(thien-3-ylcarbonyl)-1H-indol-3-yl]acetic acid derivatives having the formula: 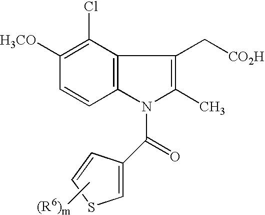
including:
[4-chloro-5-methoxy-2-methyl-1-(thien-3-ylcarbonyl)-1H-indol-3-yl]acetic acid;
{4-chloro-1-[(5-fluorothien-3-yl)carbonyl]-5-methoxy-2-methyl-1H-indol-3-yl}acetic acid;
{1-[(5-chlorothien-3-yl)carbonyl]-4-chloro-5-methoxy-2-methyl-1H-indol-3-yl}acetic acid;
{1-[(5-bromothien-3-yl)carbonyl]-4-chloro-5-methoxy-2-methyl-1H-indol-3-yl}acetic acid;
{4-chloro-5-methoxy-1-[(5-hydroxythien-3-yl)carbonyl]-2-methyl-1H-indol-3-yl}acetic acid;
{4-chloro-5-methoxy-1-[(5-methoxythien-3-yl)carbonyl]-2-methyl-1H-indol-3-yl}acetic acid;
{1-[(5-ethoxythien-3-yl)carbonyl]-4-chloro-5-methoxy-2-methyl-1H-indol-3-yl}acetic acid;
(1-{[5-(difluoromethoxy)thien-3-yl]carbonyl}-4-chloro-5-methoxy-2-methyl-1H-indol-3-yl)acetic acid;
(4-chloro-5-methoxy-2-methyl-1-{[5-(trifluoromethoxy)thien-3-yl]carbonyl}-1H-indol-3-yl)acetic acid;
(4-chloro-5-methoxy-2-methyl-1-{[5-(pentafluoroethoxy)thien-3-yl]carbonyl}-1H-indol-3-yl)acetic acid;
(4-chloro-5-methoxy-2-methyl-1-{[5-(1,1,2,2-tetrafluoroethoxy)thien-3-yl]carbonyl}-1H-indol-3-yl)acetic acid;
{4-chloro-5-methoxy-2-methyl-1-[(5-methylthien-3-yl)carbonyl]-1H-indol-3-yl}acetic acid;
(1-{[5-(difluoromethyl)thien-3-yl]carbonyl}-4-chloro-5-methoxy-2-methyl-1H-indol-3-yl)acetic acid;
(4-chloro-5-methoxy-2-methyl-1-{[5-(trifluoromethyl)thien-3-yl]carbonyl}-H-indol-3-yl)acetic acid;
(4-chloro-5-methoxy-2-methyl-1-{[5-(methylthio)thien-3-yl]carbonyl}-1H-indol-3-yl)acetic acid;
[1-({5-[(difluoromethyl)thio]thien-3-yl}carbonyl)-4-chloro-5-methoxy-2-methyl-1H-indol-3-yl]acetic acid;
[4-chloro-5-methoxy-2-methyl-1-({5-[(trifluoromethyl)thio]thien-3-yl}carbonyl)-1H-indol-3-yl]acetic acid;
[4-chloro-5-methoxy-2-methyl-1-({5-[(pentafluoroethyl)thio]thien-3-yl}carbonyl)-1H-indol-3-yl]acetic acid;
[4-chloro-5-methoxy-2-methyl-1-({5-[(1,1,2,2-tetrafluoroethyl)thio]thien-3-yl}carbonyl)-1H-indol-3-yl]acetic acid; and
{1-[(5-cyanothien-3-yl)carbonyl]-4-chloro-5-methoxy-2-methyl-1H-indol-3-yl}acetic acid.
[6-chloro-1-(cyclohexylcarbonyl)-5-methoxy-2-methyl-1H-indol-3-yl]acetic acid
[6-chloro-1-(cyclohexylcarbonyl)-5-hydroxy-2-methyl-1H-indol-3-yl]acetic acid
[1-(cyclohexylcarbonyl)-6-fluoro-5-hydroxy-2-methyl-1H-indol-3-yl]acetic acid
[1-(cyclohexylcarbonyl)-4-fluoro-5-hydroxy-2-methyl-1H-indol-3-yl]acetic acid
[4-chloro-1-(cyclohexylcarbonyl)-5-hydroxy-2-methyl-1H-indol-3-yl]acetic acid
[4-chloro-1-(cyclohexylcarbonyl)-5-methoxy-2-methyl-1H-indol-3-yl]acetic acid
[1-(cyclohexylcarbonyl)-4-fluoro-5-methoxy-2-methyl-1H-indol-3-yl]acetic acid
[4-fluoro-5-methoxy-2-methyl-1-(pyridin-2-ylcarbonyl)-1H-indol-3-yl]acetic acid
[4-fluoro-5-hydroxy-2-methyl-1-(pyridin-2-ylcarbonyl)-1H-indol-3-yl]acetic acid
[4-chloro-5-methoxy-2-methyl-1-(pyridin-2-ylcarbonyl)-1H-indol-3-yl]acetic acid
[4-chloro-5-hydroxy-2-methyl-1-(pyridin-2-ylcarbonyl)-1H-indol-3-yl]acetic acid
[6-fluoro-5-methoxy-2-methyl-1-(pyridin-2-ylcarbonyl)-1H-indol-3-yl]acetic acid
[6-fluoro-5-hydroxy-2-methyl-1-(pyridin-2-ylcarbonyl)-1H-indol-3-yl]acetic acid
[6-chloro-5-methoxy-2-methyl-1-(pyridin-2-ylcarbonyl)-1H-indol-3-yl]acetic acid
[6-chloro-5-hydroxy-2-methyl-1-(pyridin-2-ylcarbonyl)-1H-indol-3-yl]acetic acid
[5-methoxy-2-methyl-1-(pyridin-2-ylcarbonyl)-1H-indol-3-yl]acetic acid
[5-hydroxy-2-methyl-1-(pyridin-2-ylcarbonyl)-1H-indol-3-yl]acetic acid
[4-fluoro-5-methoxy-2-methyl-1-(pyridin-3-ylcarbonyl)-1H-indol-3-yl]acetic acid
[4-fluoro-5-hydroxy-2-methyl-1-(pyridin-3-ylcarbonyl)-1H-indol-3-yl]acetic acid
[4-chloro-5-methoxy-2-methyl-1-(pyridin-3-ylcarbonyl)-1H-indol-3-yl]acetic acid
[4-chloro-5-hydroxy-2-methyl-1-(pyridin-3-ylcarbonyl)-1H-indol-3-yl]acetic acid
[6-fluoro-5-methoxy-2-methyl-1-(pyridin-3-ylcarbonyl)-1H-indol-3-yl]acetic acid
[6-fluoro-5-hydroxy-2-methyl-1-(pyridin-3-ylcarbonyl)-1H-indol-3-yl]acetic acid
[6-chloro-5-methoxy-2-methyl-1-(pyridin-3-ylcarbonyl)-1H-indol-3-yl]acetic acid
[6-chloro-5-hydroxy-2-methyl-1-(pyridin-3-ylcarbonyl)-1H-indol-3-yl]acetic acid
[5-methoxy-2-methyl-1-(pyridin-3-ylcarbonyl)-1H-indol-3-yl]acetic acid
[5-hydroxy-2-methyl-1-(pyridin-3-ylcarbonyl)-1H-indol-3-yl]acetic acid
[4-fluoro-5-methoxy-2-methyl-1-(pyridin-4-ylcarbonyl)-1H-indol-3-yl]acetic acid
[4-fluoro-5-hydroxy-2-methyl-1-(pyridin-4-ylcarbonyl)-1H-indol-3-yl]acetic acid
[4-chloro-5-methoxy-2-methyl-1-(pyridin-4-ylcarbonyl)-1H-indol-3-yl]acetic acid
[4-chloro-5-hydroxy-2-methyl-1-(pyridin-4-ylcarbonyl)-1H-indol-3-yl]acetic acid
[6-fluoro-5-methoxy-2-methyl-1-(pyridin-4-ylcarbonyl)-1H-indol-3-yl]acetic acid
[6-fluoro-5-hydroxy-2-methyl-1-(pyridin-4-ylcarbonyl)-1H-indol-3-yl]acetic acid
[6-chloro-5-methoxy-2-methyl-1-(pyridin-4-ylcarbonyl)-1H-indol-3-yl]acetic acid
[6-chloro-5-hydroxy-2-methyl-1-(pyridin-4-ylcarbonyl)-1H-indol-3-yl]acetic acid
[5-methoxy-2-methyl-1-(pyridin-4-ylcarbonyl)-1H-indol-3-yl]acetic acid
[5-hydroxy-2-methyl-1-(pyridin-4-ylcarbonyl)-1H-indol-3-yl]acetic acid
[4-fluoro-5-methoxy-2-methyl-1-(tetrahydro-2H-pyran-4-ylcarbonyl)-1H-indol-3-yl]acetic acid
[4-fluoro-5-hydroxy-2-methyl-1-(tetrahydro-2H-pyran-4-ylcarbonyl)-1H-indol-3-yl]acetic acid
[4-chloro-5-methoxy-2-methyl-1-(tetrahydro-2H-pyran-4-ylcarbonyl)-1H-indol-3-yl]acetic acid
[4-chloro-5-hydroxy-2-methyl-1-(tetrahydro-2H-pyran-4-ylcarbonyl)-1H-indol-3-yl]acetic acid
[6-fluoro-5-methoxy-2-methyl-1-(tetrahydro-2H-pyran-4-ylcarbonyl)-1H-indol-3-yl]acetic acid
[6-fluoro-5-hydroxy-2-methyl-1-(tetrahydro-2H-pyran-4-ylcarbonyl)-1H-indol-3-yl]acetic acid
[6-chloro-5-methoxy-2-methyl-1-(tetrahydro-2H-pyran-4-ylcarbonyl)-1H-indol-3-yl]acetic acid
[6-chloro-5-hydroxy-2-methyl-1-(tetrahydro-2H-pyran-4-ylcarbonyl)-1H-indol-3-yl]acetic acid
[5-methoxy-2-methyl-1-(tetrahydro-2H-pyran-4-ylcarbonyl)-1H-indol-3-yl]acetic acid
[5-hydroxy-2-methyl-1-(tetrahydro-2H-pyran-4-ylcarbonyl)-1H-indol-3-yl]acetic acid
[4-fluoro-5-methoxy-2-methyl-1-(tetrahydro-2H-thiopyran-4-ylcarbonyl)-1H-indol-3-yl]acetic acid
[4-fluoro-5-hydroxy-2-methyl-1-(tetrahydro-2H-thiopyran-4-ylcarbonyl)-1H-indol-3-yl]acetic acid
[4-chloro-5-methoxy-2-methyl-1-(tetrahydro-2H-thiopyran-4-ylcarbonyl)-1H-indol-3-yl]acetic acid
[4-chloro-5-hydroxy-2-methyl-1-(tetrahydro-2H-thiopyran-4-ylcarbonyl)-1H-indol-3-yl]acetic acid
[6-fluoro-5-methoxy-2-methyl-1-(tetrahydro-2H-thiopyran-4-ylcarbonyl)-1H-indol-3-yl]acetic acid
[6-fluoro-5-hydroxy-2-methyl-1-(tetrahydro-2H-thiopyran-4-ylcarbonyl)-1H-indol-3-yl]acetic acid
[6-chloro-5-methoxy-2-methyl-1-(tetrahydro-2H-thiopyran-4-ylcarbonyl)-1H-indol-3-yl]acetic acid
[6-chloro-5-hydroxy-2-methyl-1-(tetrahydro-2H-thiopyran-4-ylcarbonyl)-1H-indol-3-yl]acetic acid
[5-methoxy-2-methyl-1-(tetrahydro-2H-thiopyran-4-ylcarbonyl)-1H-indol-3-yl]acetic acid
[5-hydroxy-2-methyl-1-(tetrahydro-2H-thiopyran-4-ylcarbonyl)-1H-indol-3-yl]acetic acid
[4-fluoro-5-methoxy-2-methyl-1-(piperidin-4-ylcarbonyl)-1H-indol-3-yl]acetic acid
[4-fluoro-5-hydroxy-2-methyl-1-(piperidin-4-ylcarbonyl)-1H-indol-3-yl]acetic acid
[4-chloro-5-methoxy-2-methyl-1-(piperidin-4-ylcarbonyl)-1H-indol-3-yl]acetic acid
[4-chloro-5-hydroxy-2-methyl-1-(piperidin-4-ylcarbonyl)-1H-indol-3-yl]acetic acid
[6-fluoro-5-methoxy-2-methyl-1-(piperidin-4-ylcarbonyl)-1H-indol-3-yl]acetic acid
[6-fluoro-5-hydroxy-2-methyl-1-(piperidin-4-ylcarbonyl)-1H-indol-3-yl]acetic acid
[6-chloro-5-methoxy-2-methyl-1-(piperidin-4-ylcarbonyl)-1H-indol-3-yl]acetic acid
[6-chloro-5-hydroxy-2-methyl-1-(piperidin-4-ylcarbonyl)-1H-indol-3-yl]acetic acid
[5-methoxy-2-methyl-1-(piperidin-4-ylcarbonyl)-1H-indol-3-yl]acetic acid
[5-hydroxy-2-methyl-1-(piperidin-4-ylcarbonyl)-1H-indol-3-yl]acetic acid
[4-fluoro-5-methoxy-2-methyl-1-[(1-methylpiperidin-4-yl)carbonyl]-1H-indol-3-yl]acetic acid
[4-fluoro-5-hydroxy-2-methyl-1-[(1-methylpiperidin-4-yl)carbonyl]-1H-indol-3-yl]acetic acid
[4-chloro-5-methoxy-2-methyl-1-[(1-methylpiperidin-4-yl)carbonyl]-1H-indol-3-yl]acetic acid
[4-chloro-5-hydroxy-2-methyl-1-[(1-methylpiperidin-4-yl)carbonyl]-1H-indol-3-yl]acetic acid
[6-fluoro-5-methoxy-2-methyl-1-[(1-methylpiperidin-4-yl)carbonyl]-1H-indol-3-yl]acetic acid
[6-fluoro-5-hydroxy-2-methyl-1-[(1-methylpiperidin-4-yl)carbonyl]-1H-indol-3-yl]acetic acid
[6-chloro-5-methoxy-2-methyl-1-[(1-methylpiperidin-4-yl)carbonyl]-1H-indol-3-yl]acetic acid
[6-chloro-5-hydroxy-2-methyl-1-[(1-methylpiperidin-4-yl)carbonyl]-1H-indol-3-yl]acetic acid
[5-methoxy-2-methyl-1-[(1-methylpiperidin-4-yl)carbonyl]-1H-indol-3-yl]acetic acid
[5-hydroxy-2-methyl-1-[(1-methylpiperidin-4-yl)carbonyl]-1H-indol-3-yl]acetic acid
[4-fluoro-5-methoxy-2-methyl-1-[(4-methylpiperazin-1-yl)carbonyl]-1H-indol-3-yl]acetic acid
[4-fluoro-5-hydroxy-2-methyl-1-[(4-methylpiperazin-1-yl)carbonyl]-1H-indol-3-yl]acetic acid
[4-chloro-5-methoxy-2-methyl-1-[(4-methylpiperazin-1-yl)carbonyl]-1H-indol-3-yl]acetic acid
[4-chloro-5-hydroxy-2-methyl-1-[(4-methylpiperazin-1-yl)carbonyl]-1H-indol-3-yl]acetic acid
[6-fluoro-5-methoxy-2-methyl-1-[(4-methylpiperazin-1-yl)carbonyl]-1H-indol-3-yl]acetic acid
[6-fluoro-5-hydroxy-2-methyl-1-[(4-methylpiperazin-1-yl)carbonyl]-1H-indol-3-yl]acetic acid
[6-chloro-5-methoxy-2-methyl-1-[(4-methylpiperazin-1-yl)carbonyl]-1H-indol-3-yl]acetic acid
[6-chloro-5-hydroxy-2-methyl-1-[(4-methylpiperazin-1-yl)carbonyl]-1H-indol-3-yl]acetic acid
[5-methoxy-2-methyl-1-[(4-methylpiperazin-1-yl)carbonyl]-1H-indol-3-yl]acetic acid
[5-hydroxy-2-methyl-1-[(4-methylpiperazin-1-yl)carbonyl]-1H-indol-3-yl]acetic acid
[4-fluoro-5-methoxy-2-methyl-1-(piperazin-1-ylcarbonyl)-1H-indol-3-yl]acetic acid
[4-fluoro-5-hydroxy-2-methyl-1-(piperazin-1-ylcarbonyl)-1H-indol-3-yl]acetic acid
[4-chloro-5-methoxy-2-methyl-1-(piperazin-1-ylcarbonyl)-1H-indol-3-yl]acetic acid
[4-chloro-5-hydroxy-2-methyl-1-(piperazin-1-ylcarbonyl)-1H-indol-3-yl]acetic acid [6-fluoro-5-methoxy-2-methyl-1-(piperazin-1-ylcarbonyl)-1H-indol-3-yl]acetic acid
[6-fluoro-5-hydroxy-2-methyl-1-(piperazin-1-ylcarbonyl)-1H-indol-3-yl]acetic acid
[6-chloro-5-methoxy-2-methyl-1-(piperazin-1-ylcarbonyl)-1H-indol-3-yl]acetic acid
[6-chloro-5-hydroxy-2-methyl-1-(piperazin-1-ylcarbonyl)-1H-indol-3-yl]acetic acid
[5-methoxy-2-methyl-1-(piperazin-1-ylcarbonyl)-1H-indol-3-yl]acetic acid
[5-hydroxy-2-methyl-1-(piperazin-1-ylcarbonyl)-1H-indol-3-yl]acetic acid
[4-fluoro-5-methoxy-2-methyl-1-(piperidin-1-ylcarbonyl)-1H-indol-3-yl]acetic acid
[4-fluoro-5-hydroxy-2-methyl-1-(piperidin-1-ylcarbonyl)-1H-indol-3-yl]acetic acid
[4-chloro-5-methoxy-2-methyl-1-(piperidin-1-ylcarbonyl)-1H-indol-3-yl]acetic acid
[4-chloro-5-hydroxy-2-methyl-1-(piperidin-1-ylcarbonyl)-1H-indol-3-yl]acetic acid
[6-fluoro-5-methoxy-2-methyl-1-(piperidin-1-ylcarbonyl)-1H-indol-3-yl]acetic acid
[6-fluoro-5-hydroxy-2-methyl-1-(piperidin-1-ylcarbonyl)-1H-indol-3-yl]acetic acid
[6-chloro-5-methoxy-2-methyl-1-(piperidin-1-ylcarbonyl)-1H-indol-3-yl]acetic acid
[6-chloro-5-hydroxy-2-methyl-1-(piperidin-1-ylcarbonyl)-1H-indol-3-yl]acetic acid
[5-methoxy-2-methyl-1-(piperidin-1-ylcarbonyl)-1H-indol-3-yl]acetic acid
[5-hydroxy-2-methyl-1-(piperidin-1-ylcarbonyl)-1H-indol-3-yl]acetic acid
[6-chloro-1-(cyclopentylcarbonyl)-5-methoxy-2-methyl-1H-indol-3-yl]acetic acid
[6-chloro-1-(cyclopentylcarbonyl)-5-hydroxy-2-methyl-1H-indol-3-yl]acetic acid
[1-(cyclopentylcarbonyl)-6-fluoro-5-methoxy-2-methyl-1H-indol-3-yl]acetic acid
[1-(cyclopentylcarbonyl)-6-fluoro-5-hydroxy-2-methyl-1H-indol-3-yl]acetic acid
[1-(cyclopentylcarbonyl)-4-fluoro-5-hydroxy-2-methyl-1H-indol-3-yl]acetic acid
[4-chloro-1-(cyclopentylcarbonyl)-5-hydroxy-2-methyl-1H-indol-3-yl]acetic acid
[4-chloro-1-(cyclopentylcarbonyl)-5-methoxy-2-methyl-1H-indol-3-yl]acetic acid
[1-(cyclopentylcarbonyl)-4-fluoro-5-methoxy-2-methyl-1H-indol-3-yl]acetic acid
[1-(cyclopentylcarbonyl)-5-methoxy-2-methyl-1H-indol-3-yl]acetic acid
[1-(cyclopentylcarbonyl)-5-hydroxy-2-methyl-1H-indol-3-yl]acetic aci[6-chloro-1-(cyclobutylcarbonyl)-5-methoxy-2-methyl-1H-indol-3-yl]acetic acid
[6-chloro-1-(cyclobutylcarbonyl)-5-hydroxy-2-methyl-1H-indol-3-yl]acetic acid
[1-(cyclobutylcarbonyl)-6-fluoro-5-methoxy-2-methyl-1H-indol-3-yl]acetic acid
[1-(cyclobutylcarbonyl)-6-fluoro-5-hydroxy-2-methyl-1H-indol-3-yl]acetic acid
[1-(cyclobutylcarbonyl)-4-fluoro-5-hydroxy-2-methyl-1H-indol-3-yl]acetic acid
[4-chloro-1-(cyclobutylcarbonyl)-5-hydroxy-2-methyl-1H-indol-3-yl]acetic acid
[4-chloro-1-(cyclobutylcarbonyl)-5-methoxy-2-methyl-1H-indol-3-yl]acetic acid
[1-(cyclobutylcarbonyl)-4-fluoro-5-methoxy-2-methyl-1H-indol-3-yl]acetic acid
[1-(cyclobutylcarbonyl)-5-methoxy-2-methyl-1H-indol-3-yl]acetic acid
[1-(cyclobutylcarbonyl)-5-hydroxy-2-methyl-1H-indol-3-yl]acetic acid 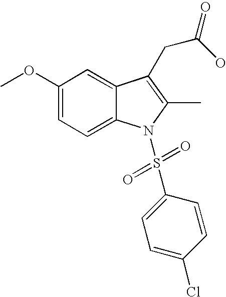
{1-[(4-chlorophenyl)sulfonyl]-5-methoxy-2-methyl-1H-indol-3-yl}acetic acid 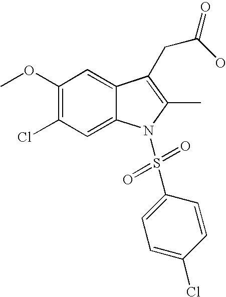
{6-chloro-1-[(4-chlorophenyl)sulfonyl]-5-methoxy-2-methyl-1H-indol-3-yl}acetic acid 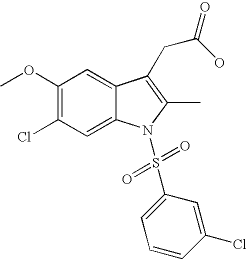
{6-chloro-1-[(3-chlorophenyl)sulfonyl]-5-methoxy-2-methyl-1 H-indol-3-yl}acetic acid 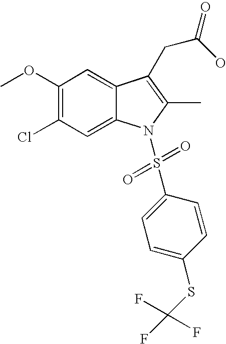
[6-chloro-5-methoxy-2-methyl-1-({4-[(trifluoromethyl)thio]phenyl}sulfonyl)-1H-indol-3-yl]acetic acid 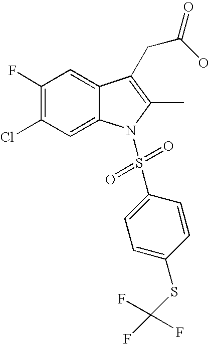
[6-chloro-5-fluoro-2-methyl-1-({4-[(trifluoromethyl)thio]phenyl}sulfonyl)-1H-indol-3-yl]acetic acid 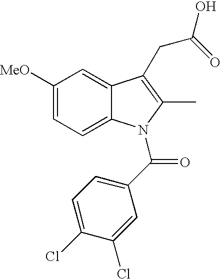
[1-(3,4-dichlorobenzoyl)-5-methoxy-2-methyl-1H-indol-3-yl]acetic acid 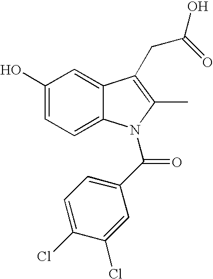
[1-(3,4-dichlorobenzoyl)-5-hydroxy-2-methyl-1H-indol-3-yl]acetic acid 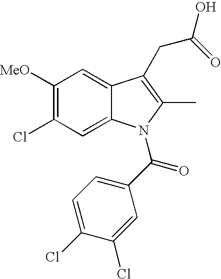
[6-chloro-1-(3,4-dichlorobenzoyl)-5-methoxy-2-methyl-1H-indol-3-yl]acetic acid 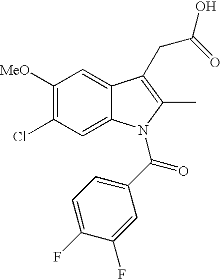
[6-chloro-1-(3,4-difluorobenzoyl)-5-methoxy-2-methyl-1H-indol-3-yl]acetic acid 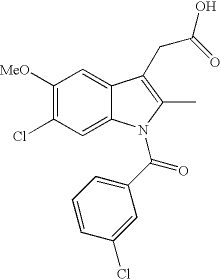
[6-chloro-1-(3-chlorobenzoyl)-5-methoxy-2-methyl-1H-indol-3-yl]acetic acid 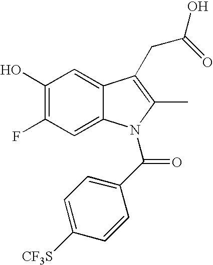
[1-(4-chlorobenzoyl)-6-fluoro-5-hydroxy-2-methyl-1H-indol-3-yl]acetic acid 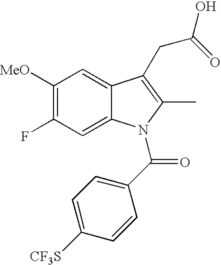
[1-(4-chlorobenzoyl)-6-fluoro-5-methoxy-2-methyl-1H-indol-3-yl]acetic acid 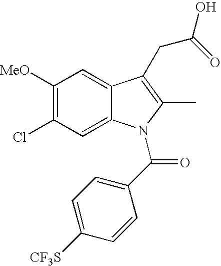
[6-chloro-1-(4-chlorobenzoyl)-5-methoxy-2-methyl-1H-indol-3-yl]acetic acid 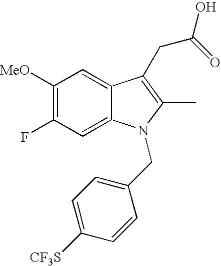
[1-(4-chlorobenzyl)-6-fluoro-5-methoxy-2-methyl-1H-indol-3-yl]acetic acid 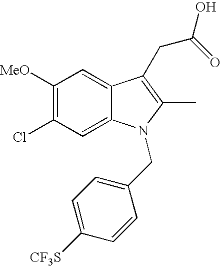
[6-chloro-1-(4-chlorobenzyl)-5-methoxy-2-methyl-1H-indol-3-yl]acetic acid 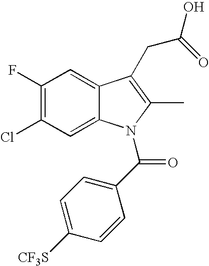
[6-chloro-1-(4-chlorobenzoyl)-5-fluoro-2-methyl-1H-indol-3-yl]acetic acid 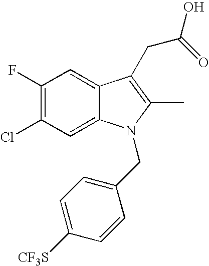
[6-chloro-1-(4-chlorobenzyl)-5-fluoro-2-methyl-1H-indol-3-yl]acetic acid 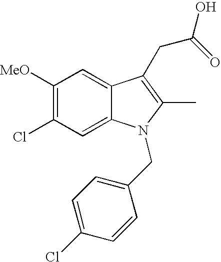
[6-chloro-1-(4-chlorobenzyl)-5-methoxy-2-methyl-1H-indol-3-yl]acetic acid 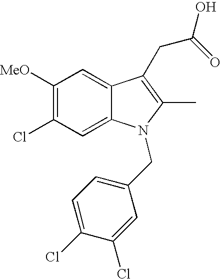
[6-chloro-1-(3,4-dichlorobenzyl)-5-methoxy-2-methyl-1H-indol-3-yl]acetic acid 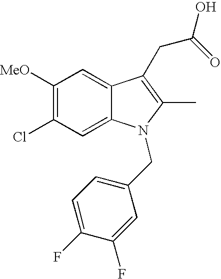
[6-chloro-1-(3,4-difluorobenzyl)-5-methoxy-2-methyl-1H-indol-3-yl]acetic acid 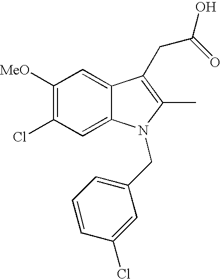
[6-chloro-1-(3-chlorobenzyl)-5-methoxy-2-methyl-1H-indol-3-yl]acetic acid 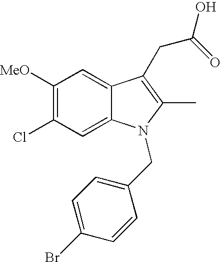
[1-(4-bromobenzyl)-6-chloro-5-methoxy-2-methyl-1H-indol-3-yl]acetic acid 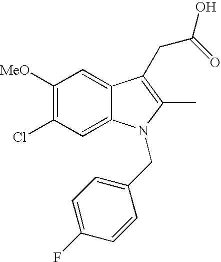
[6-chloro-1-(4-fluorobenzyl)-5-methoxy-2-methyl-1H-indol-3-yl]acetic acid 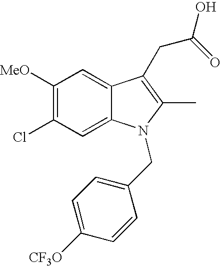
[6-chloro-1-(4-trifluoromethoxybenzyl)-5-methoxy-2-methyl-1H-indol-3-yl]acetic acid
   Synthesis Methods

### Preparation of {1-[(5-chlorothien-2-yl)carbonyl]-6-fluoro-5-hydroxy-2-methyl-1H-indol-3-yl}acetic acid

The preparation of this compound can be achieved as follows.

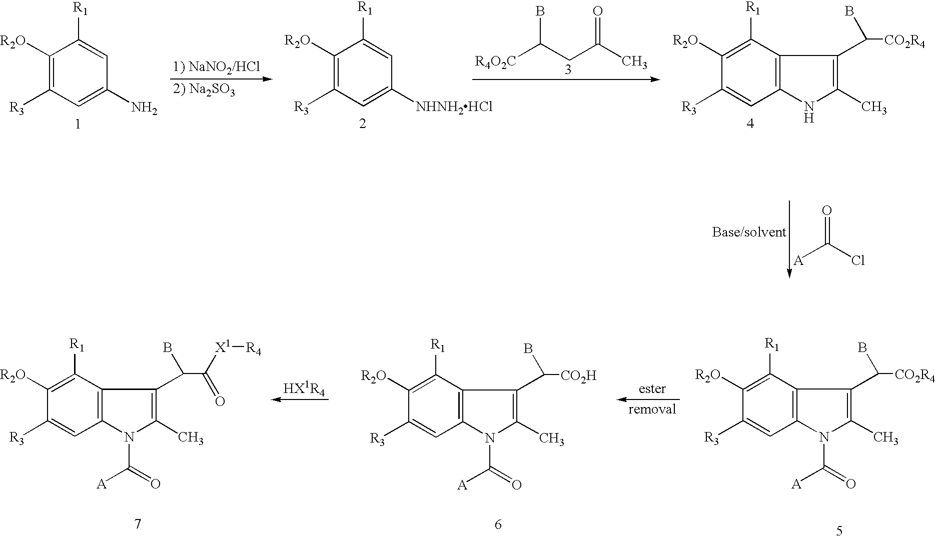

### Step 1. Preparation of (3-fluoro-4-methoxyphenyl)hydrazine (2, R₁═H, R₂═CH₃, R₃═F).

3-Fluoro-4-methoxyaniline (1, R₁═H, R₂═CH₃, R₃═F) (95 g, 0.67 mol) was added concentrated hydrochloric acid (250 mL), the suspension was stirred at ambient temperature for 18 h, then it was cooled to 0° C. and a solution of sodium nitrite (53.7 g, 0.78 mol) in water (200 mL) was added dropwise at 0-5° C. When the addition was complete, the resulting solution was stirred at 0° C. for 1 h then it was added dropwise at 0-5° C. to a stirred solution of tin (II) chloride dihydrate (638.9 g, 2.83 mol) in. concentrated hydrochloric acid (500 mL). The mixture was allowed to warm to ambient temperature then it was stored at 4° C. for 18 h. The resulting precipitate was collected by filtration, washed with water (400 mL), and ether (1000 mL) and dried in vacuo. The solid hydrochloride salt was basified by addition to 10% aqueous sodium hydroxide solution (800 mL), the free base was extracted into ether (2×400 mL), and the combined extracts were dried (MgSO₄) and the solvent removed in vacuo to give (3-fluoro-4-methoxyphenyl)hydrazine (2, R₁═H, R₂═CH₃, R₃═F) (51.9 g, 50%) as a yellow solid, mp 46-50° C.; ¹HNMR (CDCl₃/250 MHz): 1.5 (s, 1H, NH—NH₂), 3.85 (s, 3H, OCH₃), 5.0 (s, 2H, NH—NH₂), 6.44 (m, 1H, phenyl 6-H), 6.60 (dd, 1H, phenyl 5-H), 6.79 (t, 1H, phenyl 2-H).

### Step 2A. Preparation of (6-fluoro-5-methoxy-2-methyl-1H-indol-3-yl)acetic acid (4, R₁═H, R₂═CH₃, R₃═F, R₄═B═H) and (4-fluoro-5-methoxy-2-methyl-1H-indol-3-yl)acetic acid (4, R₁═F, R₂═CH₃, R₃═H, R₄═B═H)

Levulinic acid (3, B═R₄═H) (38 mL, 354 mmol) and 3-fluoro-6-methoxy-phenylhydrazine hydrochloride (2, R₁═H, R₂═CH₃, R₃═F) (67.5 g, 350 mmol) were combined and 150 mL of glacial acetic acid added and the slurry was stirred at 80° C. for 4 h. The reaction was cooled to room temperature and added to ice water (500 mL). The resulting aqueous solution was extracted with dichloromethane (3×500 mL) and the organics dried (MgSO₄) and concentrated to afford a thick semi-solid. Water (450-500 mL) was added and the slurry was stirred vigorously overnight while manually breaking up large solid pieces with a spatula. The fine tan solid that resulted was isolated by filtration and dried to afford a mixture of indoles 56.3 grams, 67% yield, ˜93% pure by HPLC (7/1 ratio of (6-fluoro-5-methoxy-2-methyl-1H-indol-3-yl)acetic acid (4, R₁═H, R₂═CH₃, R₃═F, R₄═B═H) and (4-fluoro-5-methoxy-2-methyl-1H-indol-3-yl)acetic acid (4, R₁=F, R₂═CH₃, R₃═H, R₄═B═H) of by NMR). Major isomer ¹H-NMR (CDCl₃/300 MHz) 2.27 (s, 3H), 3.82 (s, 2H), 3.84 (s, 3H), 6.92-6.97 (m, 2H, ArH).

### Step 2B. Preparation of 2-trimethylsilylethyl (6-fluoro-5-methoxy-2-methyl-1H-indol-3-yl)acetate (4. R₁═B═H, R₂═CH₃, R₃═F, R₄═CH₂CH Si(CH₃)₃)

The indoles from Step 2A (56.3 g, 238 mmol) were combined with 2-trimethylsilylethanol (41 mL, 1.25 eq.) and 4-(dimethylamino)pyridine (DMAP) (4 g) in dichloromethane (600 mL) and cooled to 0° C. 1-[3-(Dimethylamino)propyl)]-3-ethylcarbodiimide hydrochloride (EDCI) (50.2 g, 1.1 eq.) was added in portions and the reaction was stirred for 30 min at 0° C. and then allowed to warm to room temperature and stir overnight. The reaction mixture was diluted with dichloromethane (600 mL) and washed with water (2×200 mL), dried and concentrated to give a thick orange syrup which after triturating with hexanes induced solid formation, the solid was recrystallized from hexane-ethyl acetate to afford tan needles of 2-trimethylsilylethyl (6-fluoro-5-methoxy-2-methyl-1H-indol-3-yl)acetate (4, R₁═H═B, R₂═CH₃, R₃═F, R₄═CH₂CH₂Si(CH₃)₃), 52 g, 65% yield, >98% pure; ¹H-NMR (CDCl₃/300 MHz) 0.16 (s, 9H), 0.98 (m, 2H), 2.37 (s, 3H), 3.61 (s, 2H), 3.93 (s, 3H), 4.12 (m, 2H), 7.00-7.05 (m, 2H, ArH). The other regioisomer, 2-trimethylsilylethyl (4-fluoro-5-methoxy-2-methyl-1H-indol-3-yl)acetate (4, R₁═F, R₃═B═H, R₂═CH₃, R₄═CH₂CH₂Si(CH₃)₃), may be isolated by concentration of the filtrate and purification by chromatography on silica gel.

### Step 3. Preparation of 2-trimethylsilylethyl-{1-[(5-chlorothien-2-yl)carbonyl]-6-fluoro-5-methoxy-2-methyl-1H-indol-3-yl}acetate (5, R₁═H═B, R₂═CH₃, R₃═F, R₄═CH₂CH)Si(CH)₃, A=5-chlorothiophene)

In a dry flask 2-trimethylsilylethyl (6-fluoro-5-methoxy-2-methyl-1H-indol-3-yl)acetate (4, R₁═H═B, R₂═CH₃, R₃═F, R₄═CH₂CH₂Si(CH₃)₃), (1.0 g, 2.96 mmol) was dissolved in tetrahydrofuran (THF) (10 mL) and hexamethylphosphoramide (HMPA) (1 mL) and cooled to −78° C. Potassium bis(trimethylsilyl)amide 0.5M in toluene (6.52 mL) was added and the reaction was stirred for 30 min. 5-Chlorothiophene-2-carbonyl chloride (562 mg, 3.1 mmol) in 3 mL of THF was added and the reaction was stirred for 0.5 h at -78° C. and 0.5 h at 0° C., and then treated with saturated ammonium chloride (20 mL) and the reaction extracted with ethyl acetate (3×30 mL), dried over MgSO₄ and concentrated to give a thick oil which was purified by chromatography to afford 2-trimethylsilylethyl-{1-[(5-chlorothien-2-yl)carbonyl]-6-fluoro-5-methoxy-2-methyl-1H-indol-3-yl}acetate (5, R₁═H═B, R₂═CH₃, R₃═F, R₄═CH₂CH₂Si(CH₃)₃, A=5-chlorothiophene). (600 mg, 1.24 mmol, 42%, >99% pure) as light yellow oil; ¹H-NMR (CDCl₃/300 MHz) consistent with the assigned structure.

### Step 4. Preparation of {1-[(5-chlorothien-2-yl)carbonyl]-6-fluoro-5-methoxy-2-methyl-1H-indol-3-yl}acetic acid (6, R₁═H, R₂═CH₃,R₃═F, R₄═H, A=5-chlorothiophene).

A solution of the product from Step 3, 2-trimethylsilylethyl-{1-[(5-chlorothien-2-yl)carbonyl]-6-fluoro-5-methoxy-2-methyl-1H-indol-3-yl}acetate 5, R₁═H═B, R₂═CH₃, R₃═F, R₄═CH₂CH₂Si(CH₃)₃, A=5-chlorothiophene) (600 mg, 1.24 mmol) dissolved in 8 mL of THF was treated with a solution of tetrabutylammonium fluoride (1M, 3.1 mL, 3.1 mmol) in THF. The solution was stirred at room temperature until the ester had been cleaved (ca. 1 h), and then the solution was diluted with saturated aqueous ammonium chloride and extracted with ethyl acetate. The combined extracts were washed with brine, dried over MgSO₄ and concentrated to give a solid that was purified by chromatography eluting with hexanes and ethyl acetate to provide 280 mg, 59% of pure {1-[(5-chlorothien-2-yl)carbonyl]-6-fluoro-5-methoxy-2-methyl-1H-indol-3-yl}acetic acid (6, R₁═H, R₂═CH₃, R₃═F, R₄═H, A=5-chlorothiophene), mp 169° C. ¹H NMR (CDCl₃/300 MHz) 7.35 (d, 1H, J=4.0 Hz), 7.09 (d, 1H, J=11.7 Hz), 7.00 (d, 1H, J=7.2 Hz), 6.98 (d, 1H, J=4.0 Hz), 3.93 (s, 3H), 3.70 (s, 2H), 2.42 (s, 3H).

### Step 5. Preparation of {1-(5-chlorothien-2-yl)carbonyl]-6-fluoro-5-hydroxy-2-methyl-1H-indol-3-yl}acetic acid (6, R₁═H═B, R₂═H, R₃═F, A=5-chlorothiophene)

The product from Step 3, 2-trimethylsilylethyl-{I-[(5-chlorothien-2-yl)carbonyl]-6-fluoro-5-methoxy-2-methyl-1H-indol-3-yl}acetate 5, R₁═H=B, R₂═CH₃, R₃═F, R₄═CH₂CH₂Si(CH₃)₃, A=5-chlorothiophene) (400 mg. 0.83 mmol) was dissolved in 10 mL of dry dichloromethane and cooled to −78° C. The solution was then treated with boron tribromide (1M, 4.9 mL, 4.9 mmol) in dichloromethane and the solution allowed to warm to room temperature and stirred at that temperature for an additional 2 h. The solution was then poured into water and the phases separated and the aqueous phase extracted with dichloromethane. The combined extracts were washed with brine, dried over MgSO₄ and concentrated to give a solid that was purified by chromatography eluting with methanol and dichloromethane to provide 150 mg, 49%, of pure {1-[(5-chlorothien-2-yl)carbonyl]-6-fluoro-5-hydroxy-2-methyl-1H-indol-3-yl}acetic acid (6, R₁═H═B, R₂═H, R₃═F, A=5-chlorothiophene) mp 174° C., ¹H NMR (CDCl₃/300 MHz) 7.34 (d, 1H, J=3.9Hz), 7.13 (d, 1H, J=I1.1 Hz), 7.07 (d, 1H, J=8.4Hz), 6.98 (d, 1H, J=3.9 Hz), 3.66 (s, 2H), 2.39 (s, 3H).

### Step 6. Preparation on Derivatives of Compound 6

To prepare the compounds of the general formula 7, the free acid 6 can be coupled to HX¹R₄ in the presence of a dehydrating agent such as dicyclohexylcarbodiimide.

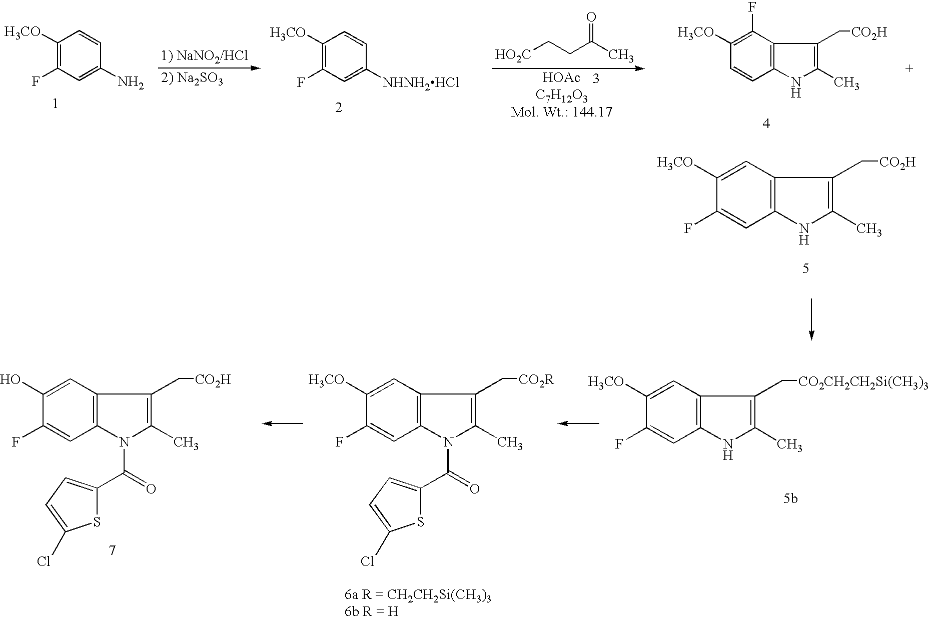

### Step 1. Preparation of phenylhydrazines, representative example: (3-fluoro-4-methoxyphenyl)hydrazine (2)

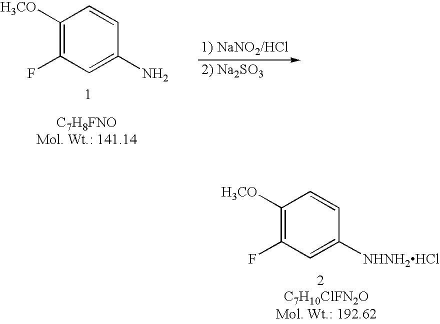

The preparation of phenylhydrazine derivatives (2) begins with treatment of commercially available anilines (1) with nitrous acid, generated from sodium nitrite and concentrated hydrochloric acid, to produce the corresponding diazonium salt. In the same reaction vessel the diazonium salt is treated with sodium sulfite and hydrochloric acid to produce the desired hydrazine hydrochloride (2) in 90% yield. Alternatively, the diazonium salt can be reduced with stannous chloride in hydrochloric acid.

### Step 2. Preparation of indoles by the Fisher Indole synthesis, representative example: (6-fluoro-5-methoxy-2-methyl-1H-indol-3-yl)acetic acid (5)

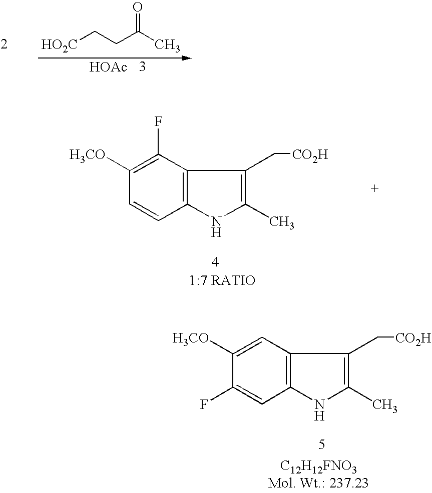

Condensation of hydrazine hydrochloride (2) with levulinic acid (3) in acetic acid results in the formation of two regioisomeric indole derivatives 4 and 5 in a 1:7 ratio. The major regioisomer 5 can be isolated in pure form by crystallization of the reaction mixture. Alternatively, the indole mixture can be esterified with an alcohol such as 2-trimethylsilylethanol to afford the corresponding esters that can then be separated by a number of means, for example by chromatography.

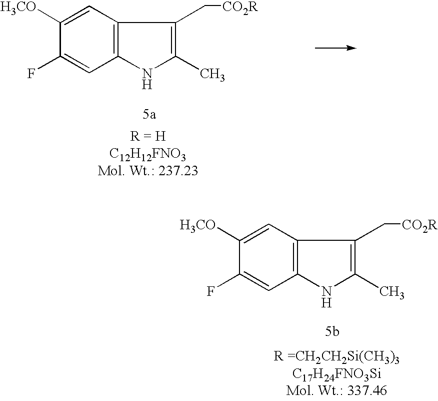

### Step 3. Acylation of indole 5b: preparation of 2-trimethylsilylethyl-{1-[(5-chlorothien-2-yl)carbonyl]-6-fluoro-5-methoxy-2-methyl-1H-indol-3-yl}acetate (6b)

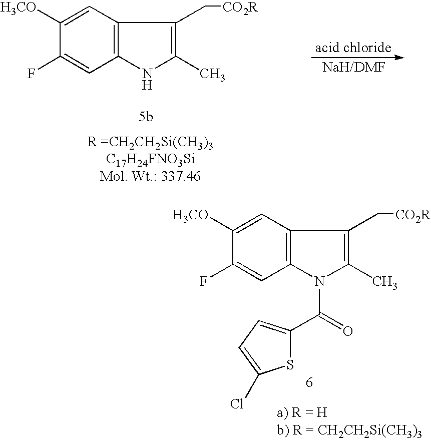

Treatment of the indole ester 5b with sodium hydride in dimethylformamide (DMF) followed by treatment with an acid chloride such as 5-chlorothiophene-2-carbonyl chloride affords the acylated indole derivative 6b in 82% yield. The ester can then be removed by treatment with an acid such as trifluoroacetic acid to produce the corresponding acid, in this instance 6a.

### Step 4. Preparation of 5-hydroxy indole derivatives: preparation of {1-[(5-chlorothien-2-yl)carbonyl]-6-fluoro-5-methoxy-2-methyl-1H-indol-3-yl}acetic acid (7)

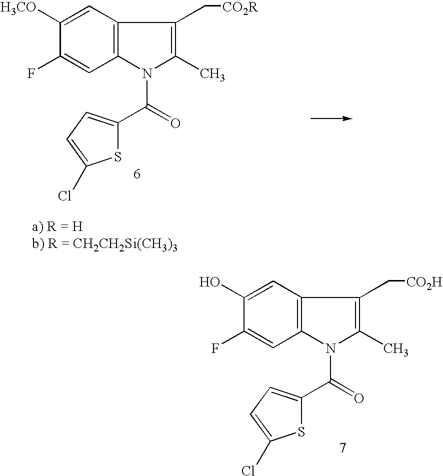

Esters such as 6b upon treatment with excess boron tribromide in dichloromethane can be converted to the corresponding acid phenols, such as 7 in good yield. Under these reaction conditions both the ester and the 5-methoxy moieties are dealkylated to the acid and phenol respectively. If desired the carboxylic acids can be converted to their salt derivatives by treatment with a base such as sodium hydroxide.

General Synthesis Scheme 2

Compounds of the invention can also be prepared according to general synthesis scheme 2 as follows.

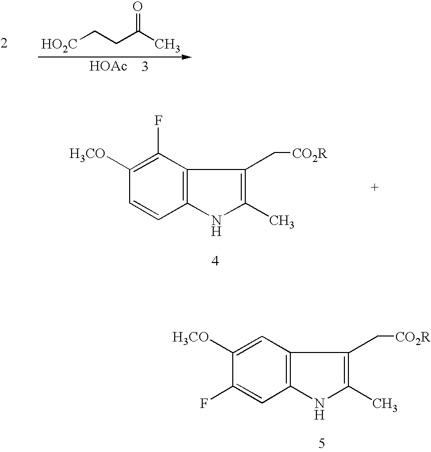

In the first step the hydrazine (2) is condensed with and ester of levulinic acid in acetic acid to afford a mixture of regioisomeric indole esters 4 and 5 (for example if one uses ethyl levulinate (3, R =Et the products (4 and 5) will be the ethyl esters, R=Et). The esters can be separated and then acylated by the procedure outline in Scheme 1 to afford the corresponding acyl derivatives such as 6, R=Et in the present example. Hydrolysis of the ester affords the corresponding acid, 6a. If desired, the ester and the 5-methoxy groups can be removed in a single operation upon treatment with boron tribromide in dichloromethane to give phenols such as 7.

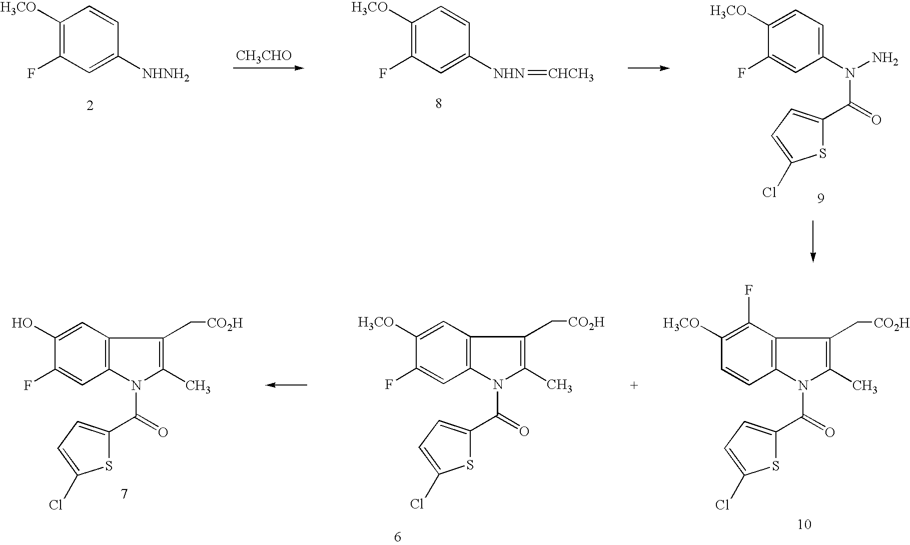

The route commences with the condensation of phenylhydrazine derivatives such as 2 with acetaldehyde to afford the corresponding hydrazone 8. Acylation of 8 with an acid chloride, in the present example 5-chlorothiophene-2-carbonyl chloride, followed by treatment with gaseous hydrochloric acid in an alcohol such as methanol provides the desired acylated hydrazine 9 after neutralization of the excess acid. Condensation of 9 with levulinic acid provides a mixture of regioisomers that can then be separated to afford acylated indoles, in the present example, 6 and 10. If desired, the 5-methoxy group can then be converted to the corresponding 5-hydroxy substituent by treatment with boron tribromide in dichloromethane for example to prepare 7.

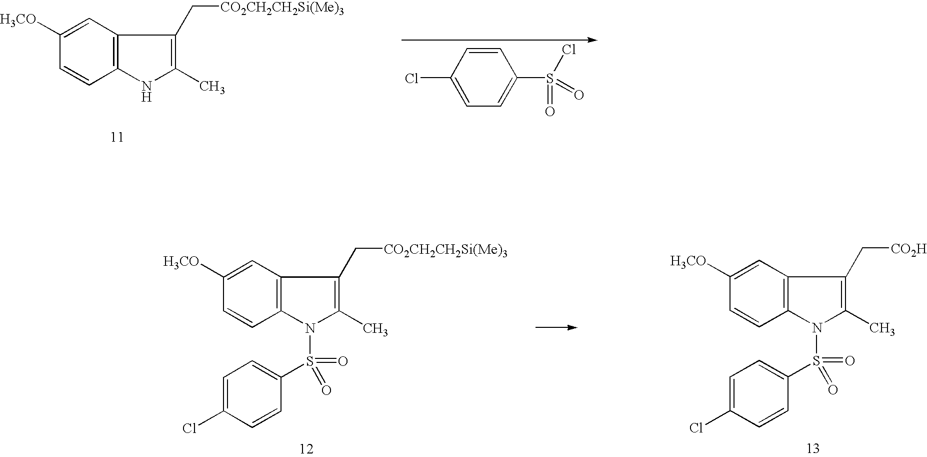

Treatment of the indole ester 11, prepared from the corresponding indole acid by coupling with 2-trimethylsilylethanol in the presence of a dehydrating agent such as dicyclohexylcarbodiimide, with a strong base such as potassium bis(trimethylsilyl)amide in tetrahydrofuran generates the indole anion that can be condensed with a sulfonyl chloride to afford the N-sulfonyl derivatives such as 12. In the present example 4-chlorobenzenesulfonyl chloride was used the sulfonyl chloride. In the second step the N-sulfonyl indole 12 is converted into the corresponding indole acid 13 upon treatment with tetrabutylammonium fluoride in tetrahydrofuran. If desired, the 5-methoxy substituent can be converted to the corresponding 5-hydroxy group upon treatment of 13 with boron tribromide in dichloromethane.

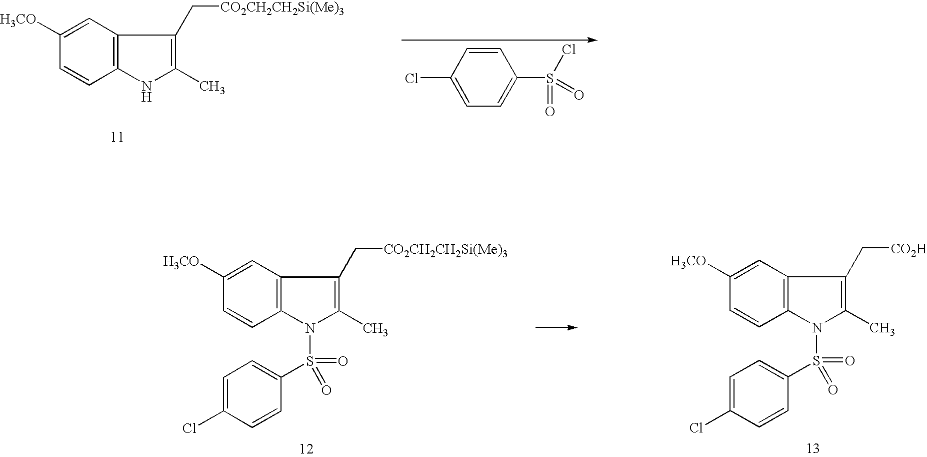

Treatment of the indole ester 11, prepared from the corresponding indole acid by coupling with 2-trimethylsilylethanol in the presence of a dehydrating agent such as dicyclohexylcarbodiimide, with a strong base such as potassium bis(trimethylsilyl)amide in tetrahydrofuran generates the indole anion that can be condensed with a cinnamoyl chloride to afford the N-acyl derivative 14. In the second step the N-acyl indole 14 is converted into the corresponding indole acid 15 upon treatment with tetrabutylammonium fluoride in tetrahydrofuran. If desired, the 5-methoxy substituent can be converted to the corresponding 5-hydroxy group, 16, upon treatment of 15 with boron tribromide in dichloromethane.

General Synthesis Scheme 6

Compounds of the invention can also be prepared according to general synthesis scheme 6 as follows

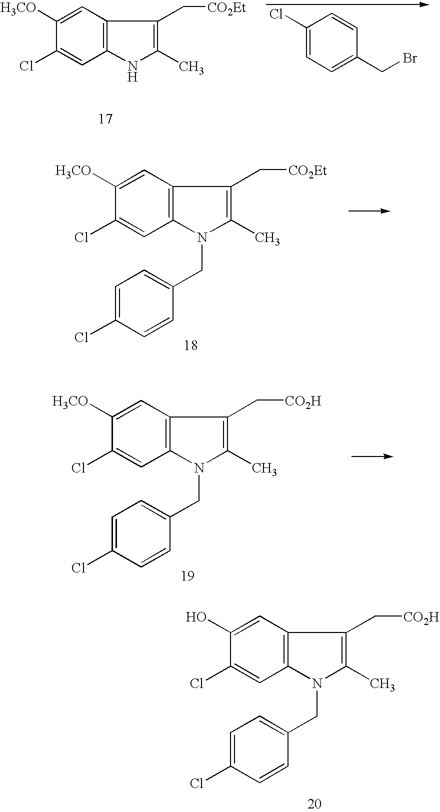

Treatment of the indole ester 17 with a strong base such as potassium bis(trimethylsilyl)amide in tetrahydrofuran generates the indole anion that can be alkylated with 4-chlorobenzyl bromide to afford the N-benzyl derivative 18. In the second step the N-benzyl indole 18 is converted into the corresponding indole acid 19 upon treatment with sodium hydroxide in aqueous tetrahydrofuran. If desired, the 5-methoxy substituent can be converted to the corresponding 5-hydroxy group, 20, upon treatment of 19 with boron tribromide in dichloromethane.

Methods for Assessing Activity In Vitro and In Vivo

Cox Related Assays

COX-1l and COX-2 Inhibition: Purified Enzyme Assays

The in vitro COX-1 and COX-2 inhibitory activity of the compounds described herein can be measured using a test kit available from Cayman Chemical (Ann Arbor, Mich.). Because COX-1 and COX-2 convert arachidonic acid to prostaglandin H₂ (PGH₂), one can assess COX inhibitory activity of a test compound by measuring the effect of the compound on PGH₂ production in the presence of purified COX-1 enzyme and in the presence of purified COX-2 enzyme. In this assay, the production of PGH₂ can be measured by reducing PGH₂ to prostaglandin F_{2α} (PGF₂,) with SnCl₂ and then detecting PGF_{2α} by enzyme immunoassay (EIA) using a suitable antibody.

COX-1 and COX-2 Inhibition: Human Whole Blood Assay

A human whole blood assay can also used to measure the inhibitory activity of each compound on COX-1 and COX-2. Briefly, human whole blood is drawn from 3-6 healthy volunteers who had not taken NSAIDS the previous 2 weeks. To measure COX-1 activity in whole blood, 100 μl of whole blood is combined with a 2 μl aliquot of test compound in vehicle or vehicle alone and incubated for 1 hr at 37° C. as described by Berg et al. (1999 Inflamm. Res. 48, 369-379). Serum is isolated from the sample by centrifugation at 12,000g for 5 min at 4° C. and then assayed for thromboxane B2 (TXB2) levels using an ELISA assay (e.g., Cayman EIA Kit, Catalog Number 519031). To measure COX-2 activity in whole blood, 100 μl of heparinized whole blood is combined with a 1 μl aliquot of 10 mg/mL LPS (lipopolysaccharide) and a 2 μl aliquot of test compound in vehicle or vehicle alone and incubated for 24 h at 37° C. as described by Berg et al. (supra). Serum is isolated from the sample by centrifugation at 12,000 g for 5 min at 4° C. and assayed for prostaglandin E₂ (PGE₂) using an ELISA assay (e.g., Cayman EIA Kit, Catalog Number 514010).

FAAH Related Assays

FAAH Inhibition: Human Whole Cell Assay and Rat and Human Brain Homogenate Assays

The ability of compounds to inhibit FAAH can be measured in human whole cell and human and rat brain homogenates as described herein.

FAAH Rat Brain Membrane (RBM) Homogenate Preparation

Adult rats (Charles River CD strain, female, 200 g) are anaesthetized with isofluorane and rapidly decapitated, respectively. Each brain is quickly removed and chilled in tubes (3 brains per tube) on ice. About 25 mL of “homogenization buffer” (20 mM HEPES buffer, pH 7.0, with 1 mM MgCl₂) is added to 15 to 20 g of brain. The brains are homogenized on ice for 1 min using an Omni GLH homogenizer (Omni International, Marietta, Ga.). The homogenates are transferred to three centrifuge tubes and centrifuged at 36,500g for 20 min at 4° C. The supernatant is discarded and each pellet is re-suspended in 25 mL “homogenization buffer”. The re-suspended material is again centrifuged (36,500 g, 20 min at 4° C.). Pellets are combined by resuspension in 10 mL of “homogenization buffer” and incubated in a 37° C. water bath for 15 min. The tubes are then placed on ice for 5 min followed by centrifugation at 36,500 g for 20 min at 4° C. The supernatant is discarded and the membrane pellets are then re-suspended in 40 mL of “resuspension buffer” (50 mM Tris-HCl buffer, pH 7.4, containing 1 mM EDTA and 3 mM MgCl₂). A Bradford Protein assay is performed to determine protein concentration. The protein is aliquotted into screw cap Cryo tubes each containing ˜400 μL, flash frozen in liquid nitrogen and stored at −80° C. until used for the assay.

FAAH Human Brain Membrane (HBM) Homogenate Preparation

About 10 g of frozen normal human brain cortex tissue is obtained (e.g., from (Analytical Biological Services (ABS), Inc. (Wilmington, Del.)). The brain tissue is thawed and transferred to a large ceramic mortar on ice. 50 mL of ice-cold “homogenization buffer” (20 mM HEPES buffer, pH 7.0, with 1 mM MgCl₂) is added to the mortar and the tissue is homogenized with a pestle. The homogenate is centrifuged at 36,500 g for 20 min at 4 ° C. The supernatants are discarded and the pellets are re-suspended in “homogenization buffer” and centrifuged as before. The supernatants are again discarded and the pellets are re-suspended in 30 mL homogenization buffer and incubated in a 37° C. water bath for 20 min. The homogenate is then centrifuged as before. The supernatant is discarded and the membrane pellets are re-suspended in 30 mL “resuspension buffer” (50 mM Tris-HCl buffer, pH 7.4, containing 1 mM EDTA and 3 mM MgCl₂). A Bradford Protein assay is performed to determine protein concentration. The protein is aliquotted into screw cap Cryo tubes each containing ˜200 μL, flash frozen in liquid nitrogen and stored at −80° C. until used for the assay.

FAAH Human Carcinoma Cell Membrane (HCM) Homogenate Preparation

Human breast epithelial carcinoma MCF7 cells are obtained from the American Type Culture Collection (ATCC Number HTB-22, Manassas, Va.) and cultured as essentially as described by ATCC. Briefly, cells are grown in Eagle's Minimum Essential Medium (ATCC catalog no. 30-2003) supplemented with 4 mM L-glutamine, 10% final volume of fetal bovine serum (ATCC catalog no. 30-2020), and 0.1 mg/mL human recombinant insulin (Sigma, St. Louis, Mo.). The cells are grown in 5% carbon dioxide in air. When cells reach ˜80% confluency, adherent cells are rinsed with Hanks Balanced Salts Solution (ATCC catalog no. 30-2213), scraped into suspension and collected by centrifugation in a clinical centrifuge at room temperature. Cell pellets are then washed by resuspension in Hanks Balanced Salts Solution followed by centrifugation. Cell pellets are then flash frozen in a dry ice and ethanol bath and stored at −80° C. Cell pellets are thawed and 25 mL of homogenization buffer is added. Membrane homogenates of MCF7 cells are then prepared as described above for rat brain homogenates. A Bradford Protein assay is performed to determine the protein concentration. The protein is aliquotted into screw cap Cryo tubes each containing ˜200 μL, flash frozen in liquid nitrogen and stored at −80° C. until used for the assay.

Determination of FAAH Activity

FAAH activity is assayed in the respective homogenates (Rat brain, Human brain, or Human breast cell carcinoma MCF7 cell) using a modification of the method of Omeir et al. (1995 Life Sci 56:1999) and Fowler et al. (1997 J. Pharmacol Exp Ther 283:729). For assay of FAAH in rat brain membrane homogenates (RBM), RBM homogenates (7 μg protein in 20 μl final volume of 10 mM Tris pH 6.5) are mixed with 180 μl of a mixture of the following: 2.0 μM unlabelled anandamide, 0.03 μCi radiolabeled anandamide [ethanolamine 1-³H] (40-60 Ci/mmol, product number ART-626, American Radiolabelled Chemicals, St. Louis, Mo.), 1 mg/mL Bovine Serum Albumin (fatty acid-free BSA, electrophoresis grade, Sigma, St. Louis Mo.), 10 mM Tris-HC₁ (pH 6.5), and 1 mM EDTA in the presence and absence of inhibitors (vehicle was DMSO at a final concentration of 1%) and incubated for 10 min at 37° C. Samples are placed on ice to terminate the reactions. ³H-ethanolamine product and un-reacted ³H-anandamide substrate are separated by either: (1) using chloroform/methanol extraction or (2) by passing the reaction mixture through a glass fiber filter containing activated charcoal. Samples are extracted with chloroform/methanol by adding 0.4 mL of chloroform/methanol (1:1 v/v), vigorously mixing the samples, and separating of the aqueous and organic phases by centrifugation. Radioactivity (corresponding to FAAH-catalyzed breakdown of ³H-anandamide) found in aliquots (0.2 mL) of the aqueous phase is determined by liquid scintillation counting with quench correction. IC₅₀ values are determined as described by Jonsson et al. (2001 Br J Pharmacol 133:1263). Alternatively, reactions are purified using a modification of the solid-phase extraction method described by Wilson et al (2003 Anal Biochem 318 : 270). This method can be modified as follows: after reactions were incubated at 37° C. for 10 min and chilled on ice, the reaction mixtures are acidified by adding 10 μl of sodium phosphate solution [0.5M (pH 2.0)]. 90 μl aliquots of the acidified reaction mixtures are applied to activated charcoal (that has been previously washed with methanol as described by Wilson et al.) containing 80 μl of water on top of a glass fiber filter, centrifuged, and the radioactivity in the eluate is counted as described previously by Wilson et al.

Whole Cell Anandamide Hydrolysis Assay

FAAH activity can be assayed in whole cells using methods disclosed previously (Maccarone et al. 1998 J Biol Chem 273:32332 and Bisogno et al. 1997 J Biol Chem 272 :3315). In addition to the cell lines described in Maccarone et al. and Bisogno et al., MCF7 (ATCC designation HTB-22) and T84 (ATCC designation CCL-248) cell lines may be used in these assays.

Determination of Endogenous and Exogenous Anandamide Levels in Rat Plasma and Brain Tissue

The effects of test compounds on endogenous and exogenously dosed anandamide levels can be measured. Rats dosed with test article are sacrificed at various time points to determine the levels of anandamide both circulating and within the brain tissue. For experiments measuring exogenous levels of anandamide, the anandamide (Cayman Chemical, Ann Arbor, Mich. or Sigma Chemical, St. Louis, Mo.) is dosed (in the range of 3-30 mg/kg) intraperitoneally (IP) 30 min post dosing of test compound. Animals are sacrificed at either 15, 30, or 60 min after anandamide administration upon anesthesia administration followed by decapitation. Brains are immediately extracted and the plasma is recovered from the blood.

Anandamide is extracted from the plasma by first precipitating the proteins by adding an equal volume of cold acetone with 10 ng of d8-anadamide (Cayman Chemicals, Ann Arbor, Mich.) as an internal standard. The acetone is evaporated from the supernatant followed by an extraction with chloroform:methanol (2:1). The chloroform layer is collected and evaporated to dryness. The pellet containing the anandamide is resuspended into methanol:chloroform (3:1) and injected onto an Xterra IS 2.1×20 mm C8 column (Waters Corporation, Milford, Mass.) and followed by detection by a Waters Quattro Micro LCMSMS (Waters Corporation, Milford, Mass.). The HPLC method consists of a step gradient (mobile phase A: 10 mM ammonium hydroxide in water, mobile phase B: 20% methanol in Acetonitrile) starting at 25% B and stepping up to 90% B at 2.2 min and holding for 2 min. Quantities are measured against known standards spiked into blank plasma using MassLynx v.4.0 software (Waters Corporation, Milford, Mass.).

Levels of anandamide from brain tissue are determined as follows. Brain tissue is homogenized in ethyl acetate and water (3:1) with 10 ng of d8-anadamide (Cayman Chemicals, Ann Arbor, Mich.) as an internal standard. The ethyl acetate layer is collected and evaporated to dryness. The pellet containing anandamide is resuspended in methanol:chloroform (3:1) and analyzed by the same method as plasma and normalized against the fresh tissue weight.

CRTH2 Related Assays

CRTH2 Agonist Assay

CRTH2 agonists increase the expression of CD11b on eosinophils. Neutrophils do not express CRTH2. They do, however, express receptors for other lipid mediators, including 5-oxo-6,8,11,14-eicosatetraenoic acid (5-oxo-ETE), leukotriene B4 (LTB4), and platelet activating factor (PAF). Therefore, any increased expression of CD11b by neutrophils is likely to be caused by an activity other than activation of CRTH2. Accordingly, preferred compounds increase CD11b expression on eosinophils, but not on neutrophils.

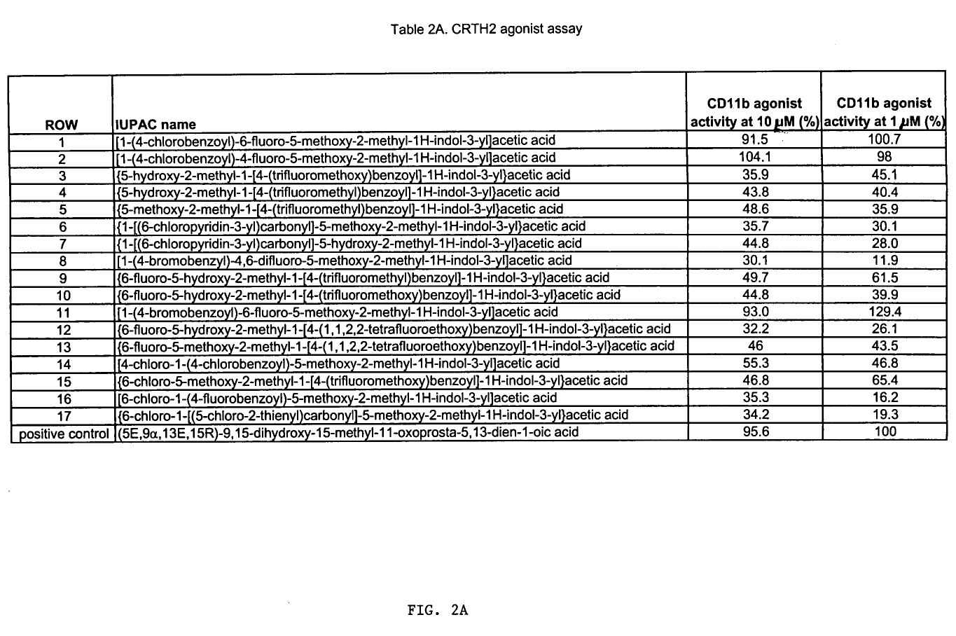

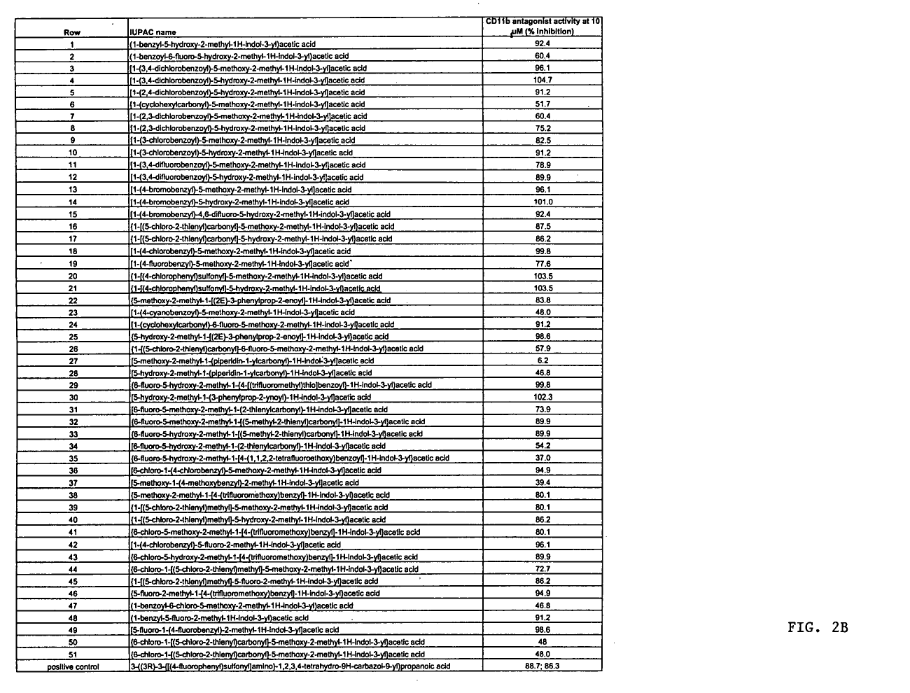

The potential CRTH2 agonist activity of certain compounds was tested using a CD11b expression assay using essentially the method described by Monneret et al. (J Pharmacol Exp Ther 304:349-55, 2003), and the results of this analysis are presented in FIG. 2a where the results of two separate experiments are reported.

Briefly, polymorphonuclear cells (0.5 ml; 10⁶/ml cells) in PBS containing 0.9 mM CaCl₂ and 0.5 mM MgCl₂) were incubated with a test compound at room temperature for 10 min. The incubations were terminated by the addition of ice-cold FACSFlow (BD Biosciences; Cat# 342003) and centrifugation (400 g for 5 min at 4° C.). The cells were then incubated for 30 min at 4° C. with a mixture of PE-labeled mouse anti-human VLA-4 (5 μl; BD Biosciences) and FITC-labeled mouse anti-human CD11b (10 μl; Beckman Coulter). The cells were then incubated with Optilyse C (0.25 ml; Beckman Coulter) for 15 min, centrifuged, and then fixed in PBS (0.4 ml; calcium and magnesium free) containing 1% formaldehyde. The distribution of fluorescence intensities among 60,000 cells was measured by flow cytometry. Eosinophils were gated out on the basis of their granularity (high side scatter) and labeling with VLA-4 (PE fluorescence). CD11b was then measured in the eosinophil region on the basis of fluorescence due to FITC. All data were corrected for the value obtained for the corresponding isotope control antibody.

The results presented in FIG. 2a are reported as the percentage of CD11b expression as compared to the maximum response generated by the positive control 15R-methyl PGD₂ ((5E,9α,13E,15R)-9,15-dihydroxy-15-methyl-11-oxoprosta-5,13-dien-1-oic acid). Compounds with greater than 30% CD11b activity in this assay were considered to be CRTH2 agonists.

To confirm that the CD11b expression is caused by activation of the CRTH2 receptor certain controls were performed. Accordingly, effect of the compounds on CD11b expression in neutrophils was tested. If the compound increases CD11b expression in neutrophils, the mobilization in eosinophils is likely caused by an activity other than activation of the CRTH2 receptor. In all cases tested CD11b expression was only observed in eosinophils.

CRTH2 Antagonist Assay

The potential CRTH2 antagonist activity of certain compounds was tested using an assay that tests the ability of the compounds to block the CD11b expression in eosinophils by 15-R-methyl-PGD₂ using essentially the method described above for the agonist assay except that the cells were preincubated with various concentrations of compounds before they were challenged with the agonist 15R-Methyl-PGD₂ The results of this analysis are presented in FIG. 2b. A CRTH2 antagonist should block CD11b expression by subsequently added 15-Methyl-PGD₂. The results presented in FIG. 2b are reported as percentage of inhibition of the maximum response generated by 15R-Methyl-PGD₂. Ramatroban (3-((3R)-3-{[(4-fluorophenyl)sulfonyl]amino}-1,2,3,4-tetrahydro-9H-carbazol-9-yl)propanoic acid), a known CRTH2 antagonist was used as a positive control in this assay. Compounds with 85% or greater inhibition in this assay are considered to be CRTH2 antagonists.

Alternatively, the CRTH2 antagonist activities can also be determined by a calcium mobilization assays conducted as follows, adapted from the protocol described by Monneret et al. (J Pharmacol Exp Ther 304:349-55,2003). Leukocytes (10⁷ cells/ml) are treated with the acetoxymethyl ester of fluo-3 (2 μM; Molecular Probes, Eugene, Oreg.) in the presence of Pluronic F-127 (0.02%; Molecular Probes) for 60 min at 23° C. The mixture is centrifuged at 200×g for 10 min and the pellet resuspended in PBS to give a concentration of 5×10⁶ cells/ml. The leukocytes are treated with PC5-labeled mouse anti-human CD16 (3.3 μl/10⁶ cells; Beckman-Coulter) for 30 min at 0° C. PBS (25 ml) is added, the mixture centrifuged as described above, and the pellet resuspended in PBS to give a concentration of 3×10⁶ leukocytes/ml. After incubation at 23° C. for 30 min, an aliquot (0.95 ml) of the leukocyte suspension is removed and treated with PBS (50 μl) containing Ca⁺⁺ (36 mM) and Mg⁺⁺ (20 mM). After 5 min, the cells are analyzed by flow cytometry using a FACSCalibur instrument (Becton-Dickinson, San Jose, Calif.). A total of approximately 10⁶ cells are counted over a period of 3 to 4 min for each sample. Fluo-3 fluorescence is measured in eosinophils, neutrophils, and monocytes, which are gated out on the basis of CD16 staining and side scatter. Test compounds are added 2 min after the start of each run followed 2 min later by 15R-Methyl-PGD₂. Maximal calcium responses are determined by addition of the calcium ionophore, A23187 (10 μM) one minute after the addition of 15R-Methyl-PGD₂.

Measurement of Pharmacokinetic Parmaters

To determine the various pharmacokinetic parameters, serum samples from animals dosed with a test compound are collected and analyzed by LCIMS. Briefly, samples are injected (10 μL) into a flow of 10% methanol in water onto a sample extraction cartridge (Waters Oasis HLB Direct Connect). The sample is washed for 0.5 min followed by a column switch that places the sample extraction cartridge into the path of the HPLC. The sample is eluted onto a reverse phase HPLC column (Waters Xterra IS C₈ 2.1×20 mm, 5 um particle size) and is eluted with a gradient (Mobile Phase A: 10 mM NH₄OH in dH₂O; Mobile Phase B: 20% methanol in Acetonitrile). Initial condition of 20% B, ramping to 90% B over 1.5 min, and holding for 0.5 min, then returning to initial conditions to re-equilibrate the column for 1 min, all at a flow rate of 0.4 mL/min.). A Micromass Quattro Micro (Waters Corp.; Milford, Mass.) triple quadrupole mass spectrometer operated in MRM mode is used to detect test compound as it elutes from the HPLC column. Concentrations are determined by relative response to an internal standard and calculated based on a standard concentration curve of the test compound. MassLynx software (Waters, Corp.; Milford, Mass.) is used to calculate the absolute concentration of test compound in each serum sample. A concentration versus time plot is generated from the data in Microsoft Excel, Summit Software PK Solutions 2.0 or Graph Pad Prism (GraphPad Software, Inc., San Diego, Calif.) to generate PK curves. From the generated curve, the AUCₙ (Area Under the Curve, n=length of experiment in h) is calculated by the software for both intravenous (IV) and orally dosed animals. Oral Bioavailability (Fₙ) is calculated using the equation: F=AUCₒᵣₐₗ/AUC_{IV}. Cₘₐₓ and Tₘₐₓ are determined by visual inspection of the oral concentration curve. Cₘₐₓ is the maximum concentration of the test compound circulating in the blood through the duration of the experiment reported at time, T (Tₘₐₓ). The terminal half-life, t_{1/2}, is calculated using at least two data points on the IV curve representing the elimination phase. Thus, the t_{1/2} is calculated by inserting the slope (β) of the line generated by plotting the natural log of the test article concentration versus time (during the elimination phase) into the equation t_{1/2}=0.693/β. The volume of distribution (Vd) is calculated using the equation Vd=Cls/β (Cls=systemic clearance, β=slope from t_{1/2} equation). Cls are determined by dividing the dose by the AUC_{IV}.

Animal Models

Animal Models For Assessing Anti-Inflammatory Activity

Any of a variety of animal models can be used to test the compounds of the invention for their effectiveness in reducing inflammation and treating pain. Useful compounds can exhibit effectiveness in reducing inflammation or pain in one or more animal models.

Carrageenan-Induced Foot Pad Edema Model

The model is described, for example, by Winter et al. (1962 Proc Soc Exp Biol Med 111:544). Briefly, rats are fasted with free access to water for 17 to 19 h before oral treatment with up to three doses of a test compound, indomethacin or celecoxib, or a control vehicle (1% methylcellulose in deionized water). One h after the last treatment, paw edema is induced by injecting 0.05 mL of a 2 % carrageenan solution into the left hindpaw.) The left hindpaw volume of each rat is measured using a plethysmometer before oral treatment, at the time of carrageenan injection and at 1.5 h, 3 h, 4.5 h after the injection of carrageenan. The edema volume of each rat at each time point is expressed as the change from the volume at the time of oral treatment and the anti-inflammatory effect in treated groups is expressed as % inhibition compared to the vehicle only group 1.5 h, 3 h and 4.5 h after the carrageenan injection. The significance of the difference between in edema different groups is assessed by a one-way analysis of variance (ANOVA) followed by the non-paired Dunnett t test. In this model, hyperalgesic response and PGE₂ production can also be measured (Zhang et al. 1997 J Pharmacol and Exp Therap 283:1069).

Complete Freund's Adjuvant (CFA) Induced Arthritis Model

In this model arthritis is induced in groups of eight Lewis derived male rats weighing 160±10 g by injecting a well-ground suspension of killed Mycobacterium tuberculosis (0.3 mg in 0.1 mL of light mineral oil; Complete Freund's Adjuvant, CFA) into the subplantar region of the right hind paw on Day 1. Hind paw volumes are measured by water displacement on Days 0, 1 and 5 (right hind paw, with CFA), and on Days 0, 14 and 18 (left hind paw, without CFA); rats are weighed on Days 0 and 18. Test compounds, dissolved or suspended in 2% Tween 80, are prepared fresh daily and administered orally twice daily for 5 consecutive days (Day 1 through Day 5) beginning one h before injection of CFA. For CFA-injected vehicle control rats, the increase in paw volume on Day 5 relative to Day 1 (Acute Phase of inflammation) is generally between 0.7 and 0.9 mL; and, that on Day 18 relative to day 14 (Delayed Phase of inflammation) is generally between 0.2 and 0.4 mL. Thus, anti-inflammatory activity in this model may be denoted by values calculated during the Acute Phase as well as the Delayed Phase. Animals are also weighed on Day 0 and Day 18; CFA-injected vehicle control animals generally gain between 40 to 60 g body weight over this time period. A 30 percent or more reduction in paw volume relative to vehicle treated controls is considered of significant anti-inflammatory activity. The mean ±SEM for each treatment group is determined and a Dunnett test is applied for comparison between vehicle and treated groups. Differences are considered significant at P<0.05. Polyarthritis of fore paw, tail, nose and ear can be scored visually and noted on the first day and final day, wherein positive (+) sign is for swelling response and negative (−) sign is normal. X-ray radiographies of the hindpaws can also be performed for further radiological index determination of arthritic symptoms. Hyperalgesia can also be measured in this model, allowing determination of analgesic effects of test compounds (Bertorelli et al. 1999 Brit Journ Pharmacol 128:1252).

Air-Pouch Model

This model is described by Masferrer et al. (1994 Proc Natl Acad Sci USA 91:3228). Briefly, male Lewis rats (175-200 g, Harlan Sprague-Dawley) are subcutaneously injected with 20 mL of sterile air into the intrascapular area of the back to create air cavities. An additional 10 mL of air is injected into the cavity every 3 days to keep the space open. Seven days after the initial air injection, 2 mL of a 1% solution of carrageenan dissolved in sterile saline is injected directly into the pouch to produce an inflammatory response. In treated and untreated animals the volume of exudate is measured and the number of leukocytes present in the exudate is determined by Wright-Giemsa staining. In addition, PGE₂ and 6-keto-PGF_{1α} are determined in the pouch exudates from treated and untreated animals by specific ELISAs (Cayman Chemicals, Ann Arbor, Mich.).

Animal Models for Assessing Analgesic Activity

Carrageenan-Induced Thermal Hyperalgesia

This model is described by Hargreaves et al. (1988 Pain 32:77). Briefly, inflammation is induced by subplantar injection of a 2% carrageenan suspension (0.1 mL) into the right hindpaw. Three h later, the nociceptive threshold is evaluated using a thermal nociceptive stimulation (plantar test). A light beam (44% of the maximal intensity) is focused beneath the hindpaw and the thermal nociceptive threshold is evaluated by the paw flick reaction latency (cut-off time: 30 sec). The pain threshold is measured in ipsilateral (inflamed) and in contralateral (control) hindpaws, 1 h after the oral treatment with the test compound or a control. The results can be expressed as the nociceptive threshold in seconds (sec) for each hindpaw and the percentage of variation of the nociceptive threshold (mean ±SEM) for each rat from the mean value of the vehicle group. A comparison of the nociceptive threshold between the inflamed paw and the control paw of the vehicle-treated group is performed using a Student's t test, a statistically significant difference is considered for P<0.05. Statistical significance between the treated groups and the vehicle group is determined by a Dunnett's test using the residual variance after a one-way analysis of variance (P<0.05) using SigmaStat Software.

Phenylbenzoquinone-Induced Writhing Model

This model is described by Siegmund et al. (1957 Proc Soc Exp Bio Med 95:729). Briefly, one h after oral dosing with a test compound, morphine or vehicle, 0.02% phenylbenzoquinone (PBQ) solution (12.5 mL/kg) is injected by intraperitoneal route into the mouse. The number of stretches and writhings are recorded from the 5th to the 10th min after PBQ injection, and can also be counted between the 35^{th} and 40^{th} min and between the 60^{th} and 65^{th} min to provide a kinetic assessment. The results are expressed as the number of stretches and writhings (mean ±SEM) and the percentage of variation of the nociceptive threshold calculated from the mean value of the vehicle-treated group. The statistical significance of any differences between the treated groups and the control group is determined by a Dunnett's test using the residual variance after a one-way analysis of variance (P<0.05) using SigmaStat Software.

Kaolin-Induced Arthritis Model.

This model is described by Hertz et al. (1980 Arzneim Forsch 30:1549). Briefly, arthritis is induced by injection of 0.1 mL of kaolin suspension into the knee joint of the right hind leg of a rat. Test compounds are administered subcutaneously after 15 min and again after two h. Reference compounds can be administered orally or subcutaneously. Gait is assessed every h from 1.5 h to 5.5 h after treatment and is scored as follows: normal gait (O), mild disability (1), intermittent raising of paw (2), and elevated paw (3). Results are expressed as the mean gait score (mean ±SEM) calculated from individual values at each time point and the percentage of variation of the mean score calculated from the mean value of the vehicle-treated group at 4.5 h and 5.5 h after treatment. The statistical significance of differences between the treated groups and the vehicle-treated group is determined by a Dunnett's test using the residual variance after a one-way analysis of variance (P<0.05) at each time point.

Peripheral Mononeuropathy Model

This model is described by Bennett et al. (1988 Pain 33:87) and can be used to assess anti-hyperalgesic effect of an orally administered test compound in a model of peripheral mononeuropathy. The effect of the test substance can be compared to a no treatment control or reference substance, e.g., morphine. Peripheral mononeuropathy is induced by loose ligation of the sciatic nerve in anaesthetized male Sprague Dawley rats (pentobarbital; 45 mg/kg by intraperitoneal route). Fourteen days later, the nociceptive threshold is evaluated using a mechanical nociceptive stimulation (analgesimeter paw pressure test; Ugo Basile, Italy). The test and reference compounds and the vehicle are orally administered (10 mL/kg carried 1% methylcellulose). Increasing pressure is applied to the hindpaw of the animal until the nociceptive reaction (vocalization or paw withdrawal) is reached. The pain threshold (grams of contact pressure) is measured in ipsilateral (injured) and in contralateral (non injured) hindpaws, 60 min after treatment. The results are expressed as: the nociceptive threshold (mean ±SEM) in grams of contact pressure for the injured paw and for the non-injured paw (vehicle-treated group) and the percentage of variation the nociceptive threshold calculated from the mean value of the vehicle-treated group. A comparison of the nociceptive threshold between the non injured paw and the injured paw of the vehicle-treated group is performed using a Student's t test. The statistical significance of the difference between the treated groups and the vehicle group is determined for the injured hindpaw by a Dunnett's test using the residual variance after a one-way analysis of variance (P<0.05) using SigmaStat Software (SigmaStat® v. 2.0.3 (SPSS Science Software, Erkrath GmbH)).

Diabetic Neuropathy Paw Pressure Test

Complete protocol details can be found in Rakieten et al. (1963 Cancer Chemother Rep 29:91). Briefly, diabetes is induced by intraperitoneal injection of streptozotocin in rats. Three weeks later, the nociceptive threshold is measured using the paw pressure test to assess hyperalgesia. Test compound or controls are administered intraperitoneally 30 min prior to pain measurement.

Acetic Acid Writhing Test

Briefly, a test compound is administered orally one h before intraperitoneal injection of acetic acid (0.5%, 10 mL/kg) in rats. Reduction in the number of writhes by 50 percent or more (≧50) per group of animals observed during the 5 to 11 min period after acetic acid administration, relative to a vehicle treated control group, indicates possible analgesic activity. This assay is based on that described in Inoue, K. et al. (1991 Arzneim. Forsch./Drug Res. 41: 235).

Formalin Test

Complete protocol details can be found in Hunskaar et al. (1985 Neurosci. Meth. 14:69). Briefly, 30 min after intraperitoneal administration of a test compound or a control, 20 82 l of a 5% formalin solution is injected by subplantar route into the right hindpaw of the rat. Hindpaw licking time is recorded during the early phase and the later phase after formalin injection.

Tail Flick Test

Complete protocol details can be found in D'Amour and Smith (1941 J Pharmacol. Exp Ther. 72:74). Briefly, 30 min after intraperitoneal administration of a test compound or a control, a light beam is focused onto the tail of the rat. The nociceptive reaction latency, characterized by tail withdrawal, is recorded. The cutoff time is set to 15 seconds.

Tail Immersion Test

In this test the tail of the rat is immersed into a 50-60° C. water bath. The nociceptive reaction latency, characterized by tail withdrawal, is measured (Haubrich et al. 1990 J Pharmacol Exp Ther 255:511 and Lichtman et al. 2004 Pain 109:319).

Hot Plate Test

Complete protocol details can be found in Eddy et al. (1950 J Pharmacol. Exp. Ther. 98:121). Briefly, 30 min after intraperitoneal administration of a test compound or a control, the mouse is placed on a metallic hot plate maintained at 52° C. The nociceptive reaction latency, characterized by a licking reflex of the forepaws or by a jumping off the hot plate is recorded. The cut-off time is set to 30 seconds.

Assays for Assessing Anxiolytic Activity

Compounds of the invention that modulate FAAH activity, and thus fatty acid amide levels, may also have anxiolytic activity. Animal models to assess anxiolytic activity include:

Elevated Plus Maze

The elevated plus maze consists of four maze arms that originate from a central platform, effectively forming a plus sign shape as described in van Gaalen and Steckler (2000 Behavioural Brain Research 115 :95). The maze can be made of plexiglas and is generally elevated. Two of the maze arms are unwalled (open) and two are walled (closed). The two open arms are well lit and the two enclosed arms are dark (Crawley 2000 What's Wrong With My Mouse?: Behavioral Phenotyping of Transgenic and Knockout Mice. Wiley-Liss, New York). The test is premised on the naturalistic conflict between the tendency of an animal to explore a novel environment and the aversive properties of a brightly lit, open area (Pellow et al. 1985 J. Neuroscience Methods. 14:149).

Complete protocol details can be found in Fedorova et al. (2001 J. Pharm. Exp. Ther. 299: 332). Briefly, 15 min following intraperitoneal administration of test compound or control, an animal is placed individually on the central platform, facing one of the open arms opposite to the observer. The number of open and closed arm entries, and the time spent in the different compartments of the maze by the animal (central platform, open and closed arms) is scored (as described in Gaalen et al. (supra)). An arm visit is recorded when an animal moves all four paws into the arm as described in Simonin et al. (1998 EMBO J. 17: 886). Behavior is scored by an observer and/or via a video camera over a 5-min test session. A greater amount of time spent or entries made by the animal in the open versus the closed arms is an indicator of anxiolytic activity.

Elevated Zero Maze

The elevated zero maze is a modification of the elevated plus maze. The elevated zero maze consists of a plexiglas apparatus in the shape of a circle (i.e., a circular runway of 46 cm diameter and 5.5 cm runway width) with two open and two wall-enclosed sectors of equal size. It is elevated up to a meter above the ground. This apparatus is described in Simonin et al. (supra) and Crawley (supra).

Complete protocol details can be found in Kathuria et al (2003 Nature Medicine 9: 76). Briefly, 30 min following intraperitoneal administration of test compound or control, an animal is placed on one open sector in front of an enclosed sector. Time in a new sector is recorded as entry with all four paws. Behavior will be scored by an observer and/or via a video camera over a 5-min test session. A greater amount of time spent or entries made by the animal in the open versus the walled sector is an indicator of anxiolytic activity.

Any of a variety of animal models can be used to test the compounds of the invention for their effectiveness in reducing allergic and inflammatory activity. Useful compounds can exhibit effectiveness in reducing allergic response and inflammation in one or more animal models.

Systemic Eosinophilia

The model is described, for example, by Shichijo et al. (2003 J. Pharmacol. Exp. Ther. 307:519-520). Briefly, seven week old male Brown Norway or Wistar rats are intravenously injected with 250-300 ug/rat of 13, 14-dihydro-15-keto-prostaglandin D₂ (DK-PGD₂), a CRTH2 agonist (dissolved in ethanol and PBS), or the corresponding volume of solvent. Rats are pretreated with or without intravenously injected 3-30 mg/kg Ramatroban [(+)-(3R)-3-(4-fluorobenzenesulfonamido)-1,2,3,4-tetra-hydrocarbazole-9-propionic acid], a CRTH2/thromboxane A₂ antagonist (dissolved in NaOH, pH-neutralized by HCl addition, and dosed in a 10% Cremophor solution). Peripheral blood is collected at 0, 1, 2, 3, 4 and 5 hours post-injection for blood smears. Following blood collection, animals are euthanized by complete bleeding and the femoral head and condoles are removed from the left femur. Total white blood cells are counted. Differential cell counts are performed on blood smears stained with May-Gruenwald's and Giemsa's solution based on standard morphologic and histological criteria.

Induction of Contact Hypersensitivity

In this model, induction of contact hypersensitivity (CHS) is created as described by Takeshita et al. (2004. Int. Immunol. 16(7):947-59). On days 0 and 1, female Balb/c mice, 7-8 weeks of age are painted onto the shaved abdominal skin with 400 μl of 0.5% fluorescein isothiocyanate (FITC) dissolved in acetone:dibutylpthalate (1:1, DBP). Six days later, mice are challenged by application of 20 μl of 0.5% FITC in DBP onto both sides of the right ear. The solvent control (DBP) is applied to the left ear. Challenge-induced increases in ear thickness are measured by an engineer's micrometer at 0, 24, 48 and 72 hours post-challenge. The CHS response is determined by challenge-induced increases in ear thickness. CHS response=[(right ear thickness post challenge−left ear thickness post challenge)−(right ear thickness pre challenge−left ear thickness pre challenge)].

To determine the presence of leukocyte infiltration, ears and back skins are fixed for 30 hours in zinc fixative at room temperature and embedded in paraffin for histological and immunohistochemical evaluation. For assessment of eosinophil peroxidase activity (EPO), skin sections are homogenized in 1 ml of ice cold buffer (0.05 M Tris-HC₁ pH 8.0 containing 0.1% Triton X-100). The tissue samples are centrifuged at 10,000 g for 20 minutes at 4° C. and supernatants are collected for measurement of EPO activity. In a 96 well microtiter plate, the substrate solution (100 μl of 10 mM o-phenlyenediamine in 0.05 M Tris-HC₁ and 4mM H₂O₂ ) is added to the 20-fold diluted homogenate in buffer (100 μl). The reaction mixture is incubated at room temperature for 1 hour before the reaction is stopped by the addition of 100 μl of 2M sulfuric acid. The microtiter plate is measured for absorbance.

Evan's Blue Test

Complete protocol details can be found in Takeshita et al. (2004. Int. Immunol. 16(7):947-59). Briefly, female Balb/c mice, 7 weeks of age are injected at two locations intradermally on their shaved backs with increasing concentrations of 0.1-10 ug/site of DK-PGD₂. This is followed by an intravenous injection of 0.25 ml of saline containing 1.25 mg of Evan's blue dye. Four hours post-dye injection, mice are euthanized and the back skin is collected. Edema severity is assessed by measuring the density of the extravasated dye. Effects of pharmacological inhibition of the inflammatory reaction to DK-PGD₂ will also be assessed by treatment with CRTH2 antagonists, such as Ramatroban.

Allergen-Induced Airway Cell Proliferation and Inflammation

Complete protocol details can be found in Eynott et al. (2003. J. Pharmacol. Ther. 304:22-29). Briefly, Brown Norway rats are sensitized on days 1, 2, and 3 with intraperitoneal injections of 1 mg/kg ovalbumin (OVA). Then they are exposed to OVA aerosol every 3^{rd} day on six occasions and on those days are treated with vehicle or compound 2 hours before and 8 hours after allergen exposure. Airway inflammatory cell accumulation and proliferation of cells by bromodeoxyuridine incorporation will be measured.

Prostaglandin D₂-Induced Eosinophilic Airway Inflammation

Complete protocol details can be found in Shiraishi et al (2004. J. Pharmacol. Ther. epub as DOI.10:1124/jpet.104.078212). Briefly, Brown Norway rats are intravenously injected with rat interleukin-5 or PBS, one hour prior to intratracheal administration of prostanoid receptor agonists. These agonists include the following; PGD₂, two CRTH₂-specific agonists, DK-PGD₂, and 11-deoxy-11-methylene-15-keto-PGD₂ (MK-PGD₂), a DP receptor-specific agonist BW 245C, a thromboxane A₂ receptor (TP)-specific agonist, I-BOP and Indomethacin. In some experiments, an orally delivered CRTH2/TP antagonist, Ramatroban, an intravenously delivered DP antagonist, BW A868C, or an intravenously delivered TP antagonist are administered two hours prior to administration of agonists. Rats are euthanized at 2, 8 and 24 hours post-agonist administration. Inflammatory cell accumulation in the trachea and lungs is recovered by bronchoalveolar lavage for cell counts and lungs are evaluated by histological examination. In a separate experiment, rats receive intravenous injection of IL-5 (0.2 ng/kg) or PBS one hour prior to intratracheal administration of PGD₂ (100 nmoles/animal) or vehicle. A peripheral blood sample is collected hourly post-dose of IL-5 for hematological evaluation.

Murine Allergic Inflammation

Complete protocol details are described in Fujitani et al. (2002 J. Immunol 168:443-449) and Matsuoka et al. (2000. Science 287: 2013-2017), Briefly, transgenic and wildtype mice are immunized with 10 μg OVA in 0.2 ml Alum on days 0 and 14. On day 21, the mice are exposed to aerosolized OVA (50mg/ml in sterile saline) for 20 minutes. On days 1 and 3 post-OVA challenge, mice are euthanized, bronchoalveolar lavaged, and the lavage fluid is assessed by differential cell counting.

Allergic Rhinitis in Anesthetized Rodents

In this model described, for example, by Arimura et al. (2001 J. Pharmacol. Ther. 298:411-419) guinea pigs are sensitized to OVA twice by inhalation of an aerosol solution of 1% OVA for 10 minutes. At 7 days after the second sensitization, the animals are anesthetized and artificially ventilated through a tracheal cannula using a respirator. Another glass cannula is inserted into the nasopharynx from the side of the larynx, and a fixed amount of air is continuously insufflated into the nasal cavity via the nasal cannula using another respirator. Insufflation pressure is monitored by a pressure transducer connected to the side arm of the nasal cannula as an indication of intranasal pressure. Nasal antigen challenge is performed by generating an aerosol of 3% OVA between the nasal cannula and the animal respirator for 3 minutes using an ultrasonic nebulizer, and then the intranasal pressure is measured for 30 minutes. Nasal secretion and the nose are collected for further evaluation.

A biphasic allergic rhinitis model in conscious guinea pigs is also fully described in Arimura et al. (2001 J. Pharmacol. Ther. 298:411-419).

Allergic Conjunctivitis Model

Complete protocol details are described in Arimura et al. (2001 J. Pharmacol. Ther. 298:411-419). Briefly, a 2.5% OVA solution is applied topically to both eyes (10 μl/eye) of conscious guinea pigs that have been sensitized as described in the “Allergic Rhinitis Model in Anesthetized Guinea Pigs” protocol above. Immediately following OVA application, Evan's blue dye (20 mg/kg i.v.) is injected as a marker of plasma exudation. The amount of Evan's blue extravasated in the conjunctiva and eyelid for 30 minutes is quantified. Independently, Histamine 0.001%, PGD₂ 0.01%, or a combination of the two are applied to the eyes of nonsensitized guinea pigs, and dye exudation is determined.

Assays for Assessing Antinociception Mechanism

Compounds can be tested to determine if they influence pathways involved in nociception. The results of such assays can be used to investigate the mechanism by which a test compound mediates its antinociceptive effect. In addition to the FAAH related assays, the following methods can be used to assess the mechanism by which a test compound mediates its antinociceptive effect.

Elevation of 3α,5α-THP

3α-hydroxy-5α-pregan-20-one (3α,5α-THP or allopregnanolone) is a pregnane steroid that acts as an agonist of the inhibitory GABA_{A} receptor subtype and is known to have both anxiolytic and analgesic effects in a variety of animal systems, with supportive evidence for a similar role in humans. Thus, compounds that elevate 3α,5α-THP may have an antinociceptive effect. The level of 3α,5α-THP in the brain of animals treated with a test compound can be measured as described by VanDoren et al. (1982 J Neuroscience 20:200) as follows. Briefly, steroids are extracted from individual cerebral cortical hemispheres dissected in ice-cold saline after euthanasia. Cortices are frozen at −80° C. until use. Samples are digested in 0.3 N NaOH by sonication and extracted three times in 3 mL aliquots of 10% (v/v) ethyl acetate in heptane. The aliquots are combined and diluted with 4 mL of heptane. The extracts are applied to solid phase silica columns (Burdick & Jackson, Muskegon, Mich.), washed with pentane, and steroids of similar polarity to 3α,5α-THP are eluted off of the column by the addition of 25% (v/v) acetone in pentane. The eluant is then dried under N₂ and steroids are redissolved in 20% (v/v) isopropanol RIA buffer (0.1 M NaH₂PO₄, 0.9 M NaCl, 0.1% w/v BSA, pH 7.0). Extraction efficiency is determined in 50 μl of the redissolved extract by liquid scintillation spectroscopy and the remaining sample is used in the determination of 3a,5Q-THP by radioimmunoassay. Reconstituted sample extracts (75 μl) and 3α,5α-THP standards (5-40,000 pg in 6.25% v/v ethanol, 31% v/v isopropyl alcohol in RIA buffer) are assayed in duplicate by the addition of 725 μl of RIA buffer, 100 μl of [³H]3α,5α-THP (20,000 dpm), and 100 μl of anti-3α,5α-THP antibody. Total binding is determined in the absence of unlabeled 3α,5α-THP, and nonspecific binding is determined in the absence of antibody. The antibody-binding reaction is allowed to equilibrate for 120 min at room temperature and is terminated by cooling the mixture to 4° C. Bound 3α,5α-THP is separated from unbound 3α,5α-THP by incubation with 300 μl of cold dextran coated charcoal (DCC; 0.04% dextran, 0.4% powdered charcoal in double-distilled H₂O) for 20 min. DCC is removed by centrifugation at 2000×g for 10 min. Bound radioactivity in the supernatant is determined by liquid scintillation spectroscopy. Sample values are compared to a concurrently run 3α,5α-THP standard curve and corrected for extraction efficiency.

Cannabinoid Receptor Binding

Compounds may exert an antinociceptive effect via binding to either or both of the cannabinoid receptors CB₁ and CB₂. CB₁ is expressed in the brain (Matsuda et al. 1990 Nature 346:561), and CB₂ is expressed by macrophages and in the spleen (Munro et al. 1993 Nature 365:61). Both of these receptors have been implicated in mediating analgesic effects through binding of agonists (see, for example, Clayton et al. 2002 Pain 96:253). Thus, test compounds can be assayed to determine whether they bind to one or both human cannabinoid receptors. An assay for CB₁ binding is described by Matsuda et al. (supra). This assay employs recombinant cells expressing CB₁. Binding to CB₂ can be determined in the same manner using recombinant cells expressing CB₂. Briefly, to measure the ability of a test compound to bind to CB₁, the binding of a labelled CB₁ ligand, e.g., [³H]WIN 55212-2 (2 nM for CB₁ and 0.8 nM for CB₂) to membranes isolated from HEK-293 cells expressing recombinant CB₁ is measured in the presence and absence of a compound. Non-specific binding is separately determined in the presence of several-fold excess of unlabelled WIN 55212-2 (5 μM for CB, and 10 μM for CB₂). The specific ligand binding to the receptors is defined as the difference between the total binding and the non-specific binding determined in the presence of an excess of unlabelled WIN 55212-2. The IC₅₀ values and Hill coefficients (n_{H}) are determined by non-linear regression analysis of the competition curves using Hill equation curve fitting. The inhibition constants (Kᵢ) are calculated from the Cheng Prusoff equation (Kᵢ=IC₅₀/(1+(L/K_{D})), where L=concentration of radioligand in the assay, and K_{D}=affinity of the radioligand for the receptor).

Therapeutic Methods

Cox and FAAH Related Therapeutic Methods

The compounds of the invention can be used, for example, to treat conditions or disorders in which it is considered desirable to reduce or eliminate COX-2 activity and/or FAAH activity. Thus, they can be used in any situation in which a COX-2 inhibitor or FAAH inhibitor is used as well as in other situations. For example, compounds of Formula I and related prodrugs can be used to treat an inflammatory disorder, including both disorders in which inflammation is considered a significant component of the disorder and those in which inflammation is considered a relatively minor component of the disorder, to treat acute and chronic pain (analgesic) and to treat fever (antipyretic). Among the inflammatory disorders that can be treated are auto-immune disorders. Disorders that can be treated with a composition comprising a compound having Formula I or Formula II and related prodrugs thereof include: arthritis (including rheumatoid arthritis, spondyloarthopathies, gouty arthritis, degenerative joint diseases (i.e. osteoarthritis), systemic lupus erythematosus, ankylosing spondylitis, acute painful shoulder, psoriatic, and juvenile arthritis), asthma, atherosclerosis, osteoporosis, bronchitis, tendonitis, bursitis, skin inflammation disorders (i.e. psoriasis, eczema, burns, dermatitis), enuresis, eosinophilic disease, gastrointestinal disorders (including inflammatory bowel disease, peptic ulcers, regional enteritis, diverticulitis, gastrointestinal bleeding, Crohn's disease, gastritis, irritable bowel syndrome and ulcerative colitis), and disorders ameliorated by a gastroprokinetic agent (i.e. ileus, for example post-operative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP)).

The compounds of the invention can also be used in the treatment of symptoms associated with influenza or other viral infections, common cold, sprains and strains, myositis, neuralgia, synovitis, injuries such as sports injuries and those following surgical and dental procedures, coagulation disorders, kidney disease (e.g., impaired renal function), ophthalmic disorders (including glaucoma, retinitis, retinopathies, uveitis and acute injury to the eye tissue), liver diseases (i.e., inflammatory liver disease including chronic viral hepatitis B, chronic viral hepatitis C, alcoholic liver injury, primary biliary cirrhosis, autoimmune hepatitis, nonalcoholic steatohepatitis and liver transplant rejection), and pulmonary inflammatory diseases (e.g., including asthma, allergic rhinitis, respiratory distress syndrome chronic bronchitis, and emphysema). Compositions comprising a compound having Formula I or Formula II and related prodrugs thereof can also be used to treat, for example, inflammation associated with: vascular diseases, migraine headaches, tension headaches, periarteritis nodosa, thyroiditis, aplastic anemia, Hodgkin's disease, sclerodoma, rheumatic fever, type I diabetes, myasthenia gravis, sarcoidosis, nephrotic syndrome, Behcet's syndrome, polymyositis, gingivitis, hypersensitivity, conjunctivitis, multiple sclerosis, and ischemia (e.g., myocardial ischemia), and the like. The compounds may be useful for treating neuroinflammation associated with brain disorders (e.g., Parkinson's disease and Alzheimer's disease) and chronic inflammation associated with cranial radiation injury. The compounds may be useful for treating acute inflammatory conditions (such as those resulting from infection) and chronic inflammatory conditions (such as those resulting from asthma, arthritis and inflammatory bowel disease). The compounds may also be useful in treating inflammation associated with trauma and non-inflammatory myalgia. The compounds can also be administered to those prior to surgery or taking anticoagulants. The compounds of the invention may reduce the risk of a thrombotic cardiovascular event which is defined as any sudden event of a type known to be caused by platelet aggregation, thrombosis, and subsequent ischemic clinical events, including thrombotic or thromboembolic stroke, myocardial ischemia, myocardial infarction, angina pectoris, transient ischemic attack (TIA; amaurosis fagax), reversible ischemic neurologic deficits, and any similar thrombotic event in any vascular bed (splanchnic, renal, aortic, peripheral, etc.).

The compounds of the invention may inhibit uterus contraction caused by hormones and prostanoid-induced smooth muscle contraction. The compounds of the invention may be useful in treating premature labor, menstrual cramps, menstrual irregularity, and dysmenorrhea.

The compounds of the invention may inhibit cellular neoplastic transformations and metastatic tumor growth. The compounds of the invention may be associated with reducing the number of adenomatous colorectal polyps. Thus, compounds and prodrugs may also be useful in reducing the risk of certain cancers, e.g., solid tumor cancers such as colon or colorectal cancer. The compounds and prodrugs may also be used in the treatment of prevention of all cancers including cancers of the bladder, cancers associated with overexpression of HER-2/neu cervix, skin, esophagus, head and neck, lung including non small-cell lung cancers, kidney, pancreas, prostate, gall bladder and bile duct and endometrial cancers, gastric cancers, gliomas, hepatocellular carcinomas, colonic adenomas, mammary cancers, ovarian cancers and salivary cancers. In addition, the compounds and prodrugs may be useful in treating large intestine cancer and prostate cancer. The compounds may also be useful in cases where the patient is at risk for cancer including oral premalignant lesions, cervical intraepithelial neoplasia, chronic hepatitis, bile duct hyperplasia, atypical adenomatous hyperplasia of lung, prostatic, intraepithelial neoplasia, bladder dysplasia, actinic keratoses of skin, colorectal adenomas, gastric metaplasia, and Barrett's esophagus.

Compounds of the invention are also useful for the treatment of cognitive disorders such as dementia, particularly degenerative dementia (including senile dementia, Alzheimer's disease (and precursors thereof), Pick's disease, Huntington's chorea, Parkinson's disease and Creutzfeldt-Jakob disease), and vascular dementia (including multiinfarct dementia), as well as dementia associated with intracranial space occupying lesions, trauma, infections and related conditions (including HIV infection), metabolism, toxins, anoxia and vitamin deficiency; and mild cognitive impairment associated with ageing, particularly Age Associated Memory Impairment.

Compounds of the invention may also prevent neuronal injury by inhibiting the generation of neuronal free radicals (and hence oxidative stress) and therefore are of use in the treatment of stroke; epilepsy; and epileptic seizures (including grand mal, petit mal, myoclonic epilepsy and partial seizures). The compounds of the invention may be useful to control or suppress seizures (including those that are chemically induced).

The compounds of the invention can be used in treatment of all varieties of pain including pain associated with a cough condition, pain associated with cancer, preoperative pain, arthritic pain and other forms of chronic pain such as post-operative pain, lumbosacral pain, musculo-skeletal pain, headache, migraine, muscle ache, lower back and neck pain, toothache and the like. The compounds of the invention are also useful for the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; neuralgia, such as post-herpetic neuralgia and trigeminal neuralgia; and pain resulting from physical trauma, amputation, cancer, toxins or chronic inflammatory conditions. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of the invention may also be of use in the treatment and/or prevention of cyclooxygenase-mediated proliferative disorders such as may occur in diabetic retinopathy and tumor angiogenesis. The compounds of the invention may be used to inhibit angiogenesis, such as occurs in wet macular degeneration.

The compounds of the invention may also be used for treating sexual behavior problems and/or improving sexual performances.

Certain compounds of the invention useful in the prevention and/or treatment of pain, in particular acute or chronic neurogenic pain, migraine, neuropathic pains including the forms associated with herpes virus and diabetes, acute or chronic pain associated with the inflammatory diseases: arthritis, rheumatoid arthritis, osteoarthritis, spondylitis, gout, vascularitis, Crohn's disease, irritable bowel syndrome and acute/sharp or chronic pains at the periphery. The compounds of the invention can also be used to prevent and/or treat emesis, dizziness, vomiting, and nausea, especially after chemotherapy, food behavioral problems/feeding disorders (i.e. eating disorders, in particular anorexias and cachexias of various natures, weight loss associated with cancer and other wasting conditions), neurological pathologies, psychiatric tremors (e.g., dyskinesias, dystonia, spasticity, obsessive compulsive behavior, Tourette's syndrome, all forms of depression and anxiety of any nature and origin, mood disturbances, psychoses), acute or chronic neurodegenerative diseases (e.g., Parkinson's disease, Alzheimer's disease, senile insanity, Huntington's chorea, lesions related to cerebral ischemia and cranial and medullary traumas, epilepsy, sleep disorders (sleep apnea), cardiovascular diseases (in particular hypertension, cardiac arrhythmias, arteriosclerosis, heart attacks, cardiac ischemias, renal ischemia), cancers (benign tumors of the skin, papillomas and cerebral tumors, prostate tumors, cerebral tumors (glioblastomas, medullary epitheliomas, medullary blastomas, neuroblastomas, tumors of origin, astrocytomas, astroblastomas, ependymomas, oligodendrogliomas, plexus tumor, neuroepithelioma, epiphysis tumor, ependyblastomas, malignant meningiomas, sarcomatosis, malignant melanomas, schwan cell cancers), disorders of the immune system (in particular autoimmune diseases including psoriasis, erythematous lupus), diseases of conjunctive or connective tissue, Sjogren's syndrome, spondylarthritis anchylosis, undifferentiated spondylarthritis undifferentiated, Behcet's disease, autoimmune hemolytic anaemias, multiple sclerosis, amyotrophic side sclerosis, amyloses, graft rejection, and illnesses affecting the blastocytes, allergic diseases (i.e., immediate or delayed hypersensitivity, allergic rhinitis or conjunctivitis, contact dermatitis), viral or bacterial parasitic infectious diseases (i.e. AIDS, meningitis), inflammatory diseases (in particular arthritic diseases: arthritis, rheumatoid arthritis osteoarthritis, spondylitis, gout, vascularitis, Crohn's disease, irritable bowel syndrome, osteoporosis, psoriasis, ocular infections and disorders (i.e. ocular hypertension, glaucoma, wet macular degeneration), lung diseases (i.e. diseases of the respiratory tracts, bronchyospasms, cough, asthma, chronic bronchitis, chronic obstruction of the respiratory tracts, emphysema), gastrointestinal disorders(i.e. irritable bowel syndrome, intestinal inflammatory disorders, ulcers, diarrheas, acid reflux), urinary incontinence, vesical inflammation, movement disorders, psychomotor disorders, hypertension, and AIDS-related complex. The compounds of the invention can be used as a sleep aid, to treat insomnia or to induce sleep. The compounds may be used to reduce or control body weight (or fat) or prevent and/or treat obesity or other appetite related disorders related to the excess consumption of food, ethanol and other appetizing substances. The compounds may be used to modulate lipid metabolism, reduce body fat (e.g., via increasing fat utilization) or reduce (or suppress) appetite (e.g., via inducing satiety). The compounds of the invention may be used to prevent, control or treat schizophrenia, paranoia or other related disorders, or other disorders of dopamine transmission.

The compounds of the invention can also be used to treat anxiety (including generalized anxiety disorder, panic disorder, and social anxiety Disorder) and depression.

CRTH2 Related Therapeutic Methods

The compounds of the invention that are CRTH2 antagonists can be used, for example, to prevent and/or treat conditions or disorders in which it is considered desirable to reduce or eliminate CRTH2 activity. The compounds of the invention that are CRTH2 agonists can be used, for example, to prevent and/or treat conditions in which it is considered desirable to: (1) downregulate CRTH2 activity via desensitization; (2) downregulate non-CRTH2 chemokine receptor activity via cross-desensitization or (3) shift the balance of Th1 and Th2 cells towards Th2 via agonism at CRTH2. CRTH2 agonists are expected to be especially useful in the prevention and/or treatment of disease and disorders characterized by an imbalance of Th1/Th2 that is shifted towards Th1 cells, e.g., rheumatoid arthritis, Type I diabetes, psoriasis, gastritis, irritable bowel disorder, multiple sclerosis, painless thyroiditis, lupus, and Crohn's Disease.

Compounds that are CRTH2 antagonists or agonists may be used to aid in preventing and/or treating a disease or disorder mediated, regulated or influenced by, for example, Th2 cells, eosinophils, basophils, platelets, Langerhans cells, dendritic cells or mast cells. They also may be used to aid in the prevention or treatment of a disease or disorder mediated, regulated or influenced by PGD₂ and metabolites thereof, such as 13,14-dihydro-15-keto-PGD₂ and 15-deoxy-Al 2,1′-PGD₂.

CRTH2 antagonists are expected to be useful in the prevention and/or treatment of disease and disorders characterized by undesirable activation of Th2 cells, eosinophils, and basophils e.g., asthma, atopic dermatitis, allergic rhinitis, allergies (e.g., food allergies, dust allergies, pollen allergies, mold allergies), and Grave's Disease. Compounds that are CRTH2 antagonists or agonists may be used to aid in preventing and/or treating the following types of diseases, conditions and disorders:
(1) respiratory tract/obstructive airways diseases and disorders including: acute-, allergic, hatrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis), antitussive activity, asthma (such as bronchial, allergic, intrinsic, extrinsic and dust asthma particularly chronic or inveterate asthma (e.g. late asthma and airways hyper-responsiveness)), bronchitis (including chronic and eosinophilic bronchitis), chronic inflammatory diseases of the lung which result in interstitial fibrosis, such as interstitial lung diseases (ILD) (e.g., idiopathic pulmonary fibrosis, or ILD associated with rheumatoid arthritis, or other autoimmune conditions), chronic obstructive pulmonary disease (COPD)(such as irreversible COPD), chronic sinusitis, conjunctivitis (e.g. allergic conjunctivitis), cystic fibrosis, fanner's lung and related diseases, fibroid lung, hypersensitivity lung diseases, hypersensitivity pneumonitis, idiopathic interstitial pneumonia, nasal congestion, nasal polyposis, otitis media, and chronic cough associated with inflammation or iatrogenic induced;
(2) systemic anaphylaxis or hypersensitivity responses, drug allergies (e.g., to penicillin, cephalosporins), insect sting allergies, and food related allergies which may have effects remote from the gut (such as migraine, rhinitis and eczema);
(3) bone and joint related diseases and disorders including: arthritis including rheumatic, infectious, autoimmune, seronegative, spondyloarthropathies (such as ankylosing spondylitis, psoriatic arthritis and Reiter's disease), osteoarthritis, and systemic sclerosis;

1(4) skin and eye related diseases and disorders including: psoriasis, atopical dermatitis, contact dermatitis, other eczmatous, dermitides, seborrheic dermatitis, cutaneous eosinophilias, chronic skin ulcers, cutaneous lupus erythematosus, contact hypersensitivity/allergic contact dermatits (including sensitivity to poison ivy, sumac, or oak), and eosinophilic folliculitis (Ofuji's disease);
(5) gastrointestinal tract related diseases and disorders including: Coeliac disease, cholecystitis, Crohn's disease, enteritis (including eosinophilic gastroenteritis), eosinophilic esophagitis, enteropathy associated with seronegative arthropathies, gastritis, inflammatory bowel disease and irritable bowel disease;
(6) transplant rejection related conditions including: acute and chronic allograft rejection following solid organ transplant, for example, transplantation of kidney, heart, liver, lung, and cornea, chronic graft versus host disease, skin graft rejection, and bone marrow transplant rejection;
(7) inflammation; and
(8) other diseases and disorders including: lupus erythematosus; systemic lupus, erythematosus; Hashimoto's thyroiditis, Grave's disease, type I diabetes, eosinophilia fasciitis, hyper IgE syndrome, idiopathic thrombocytopenia pupura; post-operative adhesions, ischemic/reperfusion injury in the heart, brain, peripheral limbs hepatitis (alcoholic, steatohepatitis and chronic viral), mastocytosis (cutaneous and systemic), mastitis (mammary gland), vaginitis, vasculitis (e.g., necrotizing, cutaneous, and hypersensitivity vasculitis), myositis (including polyinyositis, derinatomyositis), basophil related diseases including basophilic leukemia and basophilic leukocytosis, and eosinophil related diseases such as Churg-Strauss syndrome.
   Administration of Compounds

The compounds of the invention can be used alone or in combination with other compounds used to treat inflammatory disorders. Combination therapies are useful in a variety of situations, including where an effective dose of one or more of the agents used in the combination therapy is associated with undesirable toxicity or side effects when not used in combination. This is because a combination therapy can be used to reduce the required dosage or duration of administration of the individual agents.

Thus, the compounds of the invention can be used in a co-therapy with a second agent, e.g., an anti-inflammatory agent. Anti-inflammatory agents which can be used in co-therapy include: NSAIDs, 5-lipoxygenase (LO) inhibitors (e.g., masoprocol, tenidap, zileuton, pranlukast, tepoxalin, rilopirox, and flezelastine hydrochloride, enazadrem phosphate, and bunaprolast), p38 inhibitors (e.g., SB203580 and Vertex compound VX745), LTB₄ antagonists and LTA₄ hydrolase inhibitors, CRTH2 modulators (e.g., ramatroban), steroids or corticosteroids (e.g., beclomethasone, beclomethasone dipropionate, betamethasone, budesonide, bunedoside, butixocort, dexamethasone, flunisolide, fluocortin, fluticasone, hydrocortisone, methylprednisolone, mometasone, predonisolone, predonisone, tipredane, tixocortal, triamcinolone, and triamcinolone acetonide), and other compounds including: Bayer compound BAY1005 (CA registry 128253-31-6), Ciba Geigy compound CGS-25019C, Leo Denmark compound ETH-615, Lilly compound LY-293 111, Ono compound ONO-4057, Terumo compound TMK-688, Lilly compounds LY-213024, 264086 and 292728, ONO compound ONO-LB457, Searle compound SC-53228, calcitrol, Lilly compounds LY-210073, LY-223982, LY-233469, and LY-255283, ONO compound ONO-LB-448, Searle compounds SC-41930, SC-50605 and SC-51146, and SmithKline SKF-104493.

The compounds of the invention can be used in combination with selective COX-2 inhibitors, e.g., Celecoxib, Valdecoxib, Parecoxib, Rofecoxib, Etoricoxib, and Lumaricoxib.

The compounds of the invention can be used in a co-therapy with an agent used to treat an anxiety disorders, including: benzodiazepines (e.g., Xanax®, Librium®), SSRIs (e.g., Prozac®, Zoloft®), monoamine oxidase inhibitors (MAOIs) and tricyclic antidepressants (TCAs, e.g., amitryptilline).

The compounds of the invention can be used in a co-therapy with an agent used to treat rheumatoid arthritis including etanercept (Enbrel®) and infliximab (Remicade®).

The compounds of the invention can also be used in a co-therapy with a second agent that has analgesic activity. Analgesics which can be used in co-therapy include, but are not limited to: NSAIDs (e.g., acemetacin, acetaminophen, acetyl salicylic acid, alclofenac, alminoprofen, apazone, aspirin, benoxaprofen, bezpiperylon, bucloxic acid, carprofen, clidanac, diclofenac, diclofenac, diflunisal, diflusinal, etodolac, fenbufen, fenbufen, fenclofenac, fenclozic acid, fenoprofen, fentiazac, feprazone, flufenamic acid, flufenisal, flufenisal, fluprofen, flurbiprofen, flurbiprofen, furofenac, ibufenac, ibuprofen, indomethacin, indomethacin, indoprofen, isoxepac, isoxicam, ketoprofen, ketoprofen, ketorolac, meclofenamic acid, meclofenamic acid, mefenamic acid, mefenamic acid, miroprofen, mofebutazone, nabumetone oxaprozin, naproxen, naproxen, niflumic acid, oxaprozin, oxpinac, oxyphenbutazone, phenacetin, phenylbutazone, phenylbutazone, piroxicam, piroxicam, pirprofen, pranoprofen, sudoxicam,tenoxican, sulfasalazine, sulindac, sulindac, suprofen, tiaprofenic acid, tiopinac, tioxaprofen, tolfenamic acid, tolmetin, tolmetin, zidometacin, zomepirac, and zomepirac), a non-narcotic analgesic such as tramadol, an opioid or narcotic analgesic (e.g., APF112, beta funaltrexamine, buprenorphine, butorphanol, codeine, cypridime, dezocine, dihydrocodeine, diphenyloxylate, enkephalin pentapeptide, fedotozine, fentanyl, hydrocodone, hydromorphone, levorphanol, loperamide, meperidine, mepivacaine, methadone, methyl nalozone, morphine, nalbuphine, nalmefene, naloxonazine, naloxone, naltrexone, naltrindole, nor-binaltorphimine, oxycodone, oxymorphone, pentazocine, propoxyphene, and trimebutine), NK1 receptor antagonists (e.g., ezlopitant and SR-14033, SSR-241585), CCK receptor antagonists (e.g., loxiglumide), NK3 receptor antagonists (e.g., talnetant, osanetant SR-142801, SSR-241585), norepinephrine-serotonin reuptake inhibitors (NSRI; e.g., milnacipran), vanilloid receptor agonists and antagonists, cannabinoid receptor agonists (e.g., arvanil), sialorphin, compounds or peptides that are inhibitors of neprilysin, frakefamide (H-Tyr-D-Ala-Phe(F)-Phe-NH₂; WO 01/019849 Al), Tyr-Arg (kyotorphin), CCK receptor agonists (e.g., caerulein), conotoxin peptides, peptide analogs of thymulin, dexloxiglumide (the R-isomer of loxiglumide; WO 88/05774), and analgesic peptides (e.g., endomorphin-1, endomorphin-2, nocistatin, dalargin, lupron, and substance P).

In addition, certain antidepressants can be used in co-therapy either because they have analgesic activity or are otherwise beneficial to use in combination with an analgesic. Examples of such anti-depressants include: selective serotonin reuptake inhibitors (e.g., fluoxetine, paroxetine, sertraline), serotonin-norepinephrine dual uptake inhibitors, venlafaxine and nefazadone. Certain anti-convulsants have analgesic activity and are useful in co-therapy. Such anti-convulsants include: gabapentin, carbamazepine, phenytoin, valproate, clonazepam, topiramate and lamotrigine. Such agents are considered particularly useful for treatment of neuropathic pain, e.g., treatment of trigeminal neuralgia, postherpetic neuralgia, and painful diabetic neuropathy. Additional compounds useful in co-therapy include: alpha-2-adrenergic receptor agonists (e.g., tizanidine and clonidine), mexiletine, corticosteroids, compounds that block the NMDA (N-methyl-Daspartate) receptor (e.g, dextromethorphan, ketamine, and amantadine), glycine antagonists, carisoprodol, cyclobenzaprine, various opiates, nonopioid antitussive (e.g. dextromethorphan, carmiphen, caramiphen and carbetapentane), opioid antitussives (e.g. codeine, hydrocodone, metaxolone. The compounds of the invention can also be combined with inhalable gaseous nitric oxide (for treating pulmonary vasoconstriction or airway constriction), a thromboxane A2 receptor antagonist, a stimulant (i.e. caffeine), an H₂-antagonist (e.g. ranitidine), an antacid (.e.g. aluminum or magnesium hydroxide), an antiflatulent (e.g. simethicone), a decongestant (e.g. phenylephrine, phenylpropanolamine, pseudophedrine, oxymetazoline, ephinephrine, naphazoline, xylometazoline, propylhexedrine, or levodesoxyephedrine), a prostaglandin (e.g. misoprostol, enprostil, rioprostil, omoprostol or rosaprostol), a diuretic, a sedating or non-sedating histamine HI receptor antagonists/antihistamines (i.e. any compound that is capable of blocking, inhibiting, reducing or otherwise interrupting the interaction between histamine and its receptor) including but not limited to: -4 astemizole, acrivastine, antazoline, astemizole, azatadine, azelastine, astamizole, bromopheniramine, bromopheniramine maleate, carbinoxamine, carebastine, cetirizine, chlorpheniramine, chloropheniramine maleate, cimetidine, clemastine, cyclizine, cyproheptadine, descarboethoxyloratadine, dexchlorpheniramine, dimethindene, diphenhydramine, diphenylpyraline, doxylamine succinate, doxylamine, ebastine, efletirizine, epinastine, famotidine, fexofenadine, hydroxyzine, hydroxyzine, ketotifen, levocabastine, levocetirizine, levocetirizine, loratadine, meclizine, mepyramine, mequitazine, methdilazine, mianserin, mizolastine, noberastine, norastemizole, noraztemizole, phenindamine, pheniramine, picumast, promethazine, pynlamine, pyrilamine, ranitidine, temelastine, terfenadine, trimeprazine, tripelenamine, and triprolidine; a 5HT1 agonist, such as a triptan (e.g. sumatriptan or naratriptan), an adenosine A1 agonist, an EP ligand, a sodium channel blocker (e.g. lamotrigine), a substance P antagonist (e.g. an NK antagonist), a cannabinoid, a 5-lipoxygenase inhibitor, a leukotriene receptor antagonist/leukotriene antagonists/LTD4 antagonists (i.e., any compound that is capable of blocking, inhibiting, reducing or otherwise interrupting the interaction between leukotrienes and the Cys LTI receptor) including but not limited to: zafirlukast, montelukast, montelukast sodium (Singulair®), pranlukast, iralukast, pobilukast, SKB-106,203 and compounds described as having LTD4 antagonizing activity described in U.S. Pat. No. 5,565,473, a DMARD (e.g. methotrexate), a neurone stabilising antiepileptic drug, a mono-aminergic uptake inhibitor (e.g. venlafaxine), a matrix metalloproteinase inhibitor, a nitric oxide synthase (NOS) inhibitor, such as an iNOS or an nNOS inhibitor, an inhibitor of the release, or action, of tumor necrosis factor, an antibody therapy, such as a monoclonal antibody therapy, an antiviral agent, such as a nucleoside inhibitor (e.g. lamivudine) or an immune system modulator (e.g. interferon), a local anaesthetic, a known FAAH inhibitor (e.g., PMSF, URB532, URB597, or BMS-1, as well as those described in those described in WO04033652, U.S. Pat. No. 6,462,054, US20030092734, US20020188009, US20030195226, and WO04033422), an antidepressant (e.g., VPI-013), a fatty acid amide (e.g. anandamide, N-palmitoyl ethanolamine, N-oleoyl ethanolamide, 2-arachidonoylglycerol, or oleamide), arvanil, analogs of anadamide and arvanil as described in US 20040122089, and a proton pump inhibitor (e.g., omeprazole, esomeprazole, lansoprazole, pantorazole and rabeprazole).

The compound of the invention can also be used in a co-therapy with a second agent that is a cannabanoid receptor antagonist to prevent and/or treat obesity and other appetite related disorders.

Agents of the invention may also be coadministered with one or more of the following:
inactivating antibodies (e.g., monoclonal or polyclonal) to interleukins (e.g., IL-4 and IL-5 (for example see Leckie et al. 2000 Lancet 356:2144));
soluble chemokine receptors (e.g. recombinant soluble IL-4 receptor (Steinke and Borish 2001 Respiratory Research 2:66));
chemokine receptor modulators including but not limited to antagonists of CCR1 (e.g.,CP-481,715 (Gladue et al. J Biol Chem 278:40473)), CCR3 (e.g., UCB35625 (Sabroe et al. J Biol Chem 2000 275:25985), CCR5 and those described in: WO0039125A1, WO02070523A1, WO03035627A1, WO03084954A1, WO04011443A1, WO04014875A1, WO04018425A1, WO04018435A1, WO04026835A1, WO04026880A1, WO04039376A1, WO04039377A1, WO04039787A1, WO04056773A1, WO04056808A1, and WO04056809A1;

PGD₂ receptor antagonists including, but not limited to, compounds described as having PGD₂ antagonizing activity in United States Published Applications US20020022218, US20010051624, and US20030055077, PCT Published Applications W09700853, WO9825919, WO03066046, WO03066047, WO03101961, WO03101981, WO04007451, WO0178697, WO04032848, WO03097042, WO03097598, WO03022814, WO03022813, and WO04058164, European Patent Applications EP945450 and EP944614, and those listed in: Torisu et al. 2004 Bioorg Med Chem Lett 14:4557, Torisu et al. 2004 Bioorg Med Chem Lett 2004 14:4891, and Torisu et al. 2004 Bioorg & Med Chem 2004 12:4685;
VLA-4 antagonists;
immunosuppressants such as cyclosporine (cyclosporine A, Sandimmune® Neoral®), tacrolimus (FK-506, Prograf®), rapamycin (sirolimus, Rapamune®) and other FK-506 type immunosuppressants, and mycophenolate, e.g., mycophenolate mofetil (CellCept®);
β-agonists including but not limited to: albuterol (Porventil®, Salbutamol®, Ventolin®), bambuterol, bitoterol, clenbuterol, fenoterol, formoterol, isoetharine (Bronkosol®, Bronkometer®), metaproterenol (Alupent®, Metaprel®), pitbuterol (Maxair®), reproterol, rimiterol, salmeterol, terbutaline (Brethaire®, Brethine®, Bricanyl®), adrenalin, isoproterenol (Isuprel®), epinephrine bitartrate (Primatene®), ephedrine, orciprenlaine, fenoterol and isoetharine;
β2-agonist-corticosteroid combinations including but not limited to: salmeterol-fluticasone (Advair®), formoterol-budesonid (Symbicort®);
a bronchodilator including but not limited to theophylline and aminophylline
a mast cell stabilizer including but not limited to cromolyn, cromolyn sodium, nedocromil, and proxicromil
an anticholinergic including but not limited to: atropine, benztropine, biperiden, flutropium, hyoscyamine, ilutropium, ipratropium, ipratropium bromide, methscopolamine, oxybutinin, rispenzepine, scopolamine, and tiotropium;
an anti-tussive including but not limited to: dextromethorphan, codeine, and hydromorphone;
a decongestant including but not limited to: pseudoephedrine and phenylpropanolamine;
an expectorant including but not limited to: guafenesin, guaicolsulfate, terpin, ammonium chloride, glycerol guaicolate, and iodinated glycerol;
a PDE inhibitor including but not limited to filaminast, denbufyllene piclamilast, roflumilast, zardaverine, and rolipram;
a recombinant humanized monoclonal antibody including byt not limited to Omalizumab (xolair®) and talizumab (tnx-901);
a lung sufactant including but not limited to dsc-104
antithrombotic agents, such as thrombolytic agents (e.g., streptokinase, alteplase, anistreplase and reteplase), heparin, hirudin and warfarin derivatives, β-blockers (e.g., atenolol), β-adrenergic agonists (e.g., isoproterenol), ACE inhibitors and vasodilators (e.g., sodium nitroprusside, nicardipine hydrochloride, nitroglycerin and enaloprilat);
anti-diabetic agents such as insulin and insulin mimetics, sulfonylureas (e.g., glyburide, meglinatide), biguanides, e.g., metformin (Glucophage®), α-glucosidase inhibitors (acarbose), thiazolidinone compounds, e.g., rosiglitazone (Avandia®), troglitazone (Rezulin®), ciglitazone, pioglitazone (Actos®) and englitazone;
preparations of interferon beta (interferon β-I α, interferon β-I β);
gold compounds such as auranofin and aurothioglucose;
TNF inhibitors, e.g., etanercept (Enbrel®), antibody therapies such as orthoclone (OKT3), daclizumab (Zenapax®), basiliximab (Simulec®)), infliximab (Remicade®) and D2E6 TNF antibody;
lubricants or emollients such as petrolatum and lanolin, keratolytic agents, vitamin D₃ derivatives (e.g., calcipotriene and calcipotriol (Dovonex®)), PUVA, anthralin (Drithrocreme®), etretinate (Tegison®) and isotretinoin;
multiple sclerosis therapeutic agents such as interferon β-I β (Betaseron®), interferon β-I α (Avonex®), azathioprine (Imurek®, Imuran®), glatiramer acetate (Capoxone(®), a glucocorticoid (e.g., prednisolone) and cyclophosphamide; and
other compounds such as 5-aminosalicylic acid and prodrugs thereof DNA-alkylating agents (e.g., cyclophosphamide, ifosfamide), antimetabolites (e.g., azathioprine, 6-mercaptopurine, methotrexate, a folate antagonist, and 5-fluorouracil, a pyrimidine antagonist), microtubule disruptors (e.g., vincristine, vinblastine, paclitaxel, colchicine, nocodazole and vinorelbine), DNA intercalators (e.g., doxorubicin, daunomycin and cisplatin), DNA synthesis inhibitors such as hydroxyurea, DNA cross-linking agents, e.g., mitomycin C, hormone therapy (e.g., tamoxifen, and flutamide), and cytostatic agents, e.g., imatinib (ST1571, Gleevec®) and rituximab (Rituxan®).

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by min, h, days, or weeks. Thus, the two or more agents can be administered within min of each other or within 1, 2, 3, 6, 9, 12, 15, 18, or 24 h of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X—Y—X, X—X—Y, Y—X—Y, Y—Y—X, X—X—Y—Y, etc.

The agents, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

The agent can be in the form of a pharmaceutically acceptable salt. Such salts are prepared from pharmaceutically acceptable non-toxic bases including inorganic bases and organic bases. Examples of salts derived from inorganic bases include aluminum, ammonium, calcium, copper, ferric, ferrous, lithium, magnesium, manganic salts, manganous, potassium, sodium, zinc, and the like. In some embodiments, the salt can be an ammonium, calcium, magnesium, potassium, or sodium salt. Examples of salts derived from pharmaceutically acceptable organic non-toxic bases include salts of primary, secondary, and tertiary amines, benethamine, N,N′-dibenzylethylenediamine, diethylamine, 2-diethylaminoethanol, 2-dimethylaminoethanol, diethanolamine, ethanolamine, ethylenediamine, N-ethylmorpholine, N-ethylpiperidine, epolamine, glucamine, glucosamine, histidine, hydrabamine, isopropylamine, lysine, methylglucamine, meglumine, morpholine, piperazine, piperidine, polyamine resins, procaine, purines, theobromine, triethylamine, trimethylamine, tripropylamine, and trolamine, tromethamine. Examples of other salts include arecoline, arginine, barium, betaine, bismuth, chloroprocaine, choline, clemizole, deanol, imidazole,and morpholineethanol. In one embodiment are tris salts.

The agents of the invention can be administered orally, e.g., as a tablet or cachet containing a predetermined amount of the active ingredient, pellet, gel, paste, syrup, bolus, electuary, slurry, capsule; powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents of the invention can also be administered by captisol delivery technology, rectal suppository or parenterally.

Compositions of the present invention may also optionally include other therapeutic ingredients, anti-caking agents, preservatives, sweetening agents, colorants, flavors, desiccants, plasticizers, dyes, and the like. Any such optional ingredient must be compatible with the compound of the invention to insure the stability of the formulation.

The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as:
BINDERS: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and -105™, sold by FMC Corporation, Marcus Hook, Pa., USA), or mixtures thereof,
FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof,
DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof,
LUBRICANTS: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W. R. Grace Co., Baltimore, Md. USA), a coagulated aerosol of synthetic silica (Deaussa Co., Plano, Tex. USA), a pyrogenic silicon dioxide (CAB-O-SIL, Cabot Co., Boston, Mass. USA), or mixtures thereof,
ANTI-CAKING AGENTS: calcium silicate, magnesium silicate, silicon dioxide, colloidal silicon dioxide, talc, or mixtures thereof,
ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, and
COATING AGENTS: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnauba wax, microcrystalline wax, or mixtures thereof.

The agents either in their free form or as a salt can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(8-caprolactone) and poly(alkylene oxide) (U.S. 20030068384) to create a sustained release formulation. Such formulations can be used to implants that release a compound of the invention or another agent over a period of a few days, a few weeks or several months depending on the polymer, the particle size of the polymer, and the size of the implant (see, e.g., U.S. Pat. No. 6,620,422). Other sustained release formulations are described in EP 0 467 389 A2, WO 93/241150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, U.S. 20030171296, U.S. 20020176841, U.S. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO 02/058672, WO 9726015, WO 97/04744, and US20020019446. In such sustained release formulations microparticles of compound are combined with microparticles of polymer. U.S. Pat. No. 6,011,011 and WO 94/06452 describe a sustained release formulation providing either polyethylene glycols (where PEG 300 and PEG 400 are most preferred) or triacetin. WO 03/053401 describes a formulation which may both enhance bioavailability and provide controlled release of the agent within the GI tract. Additional controlled release formulations are described in WO 02/38129, EP 326 151, U.S. Pat. No. 5,236,704, WO 02/30398, WO 98/13029; U.S. 20030064105, U.S. 20030138488A1, U.S. 20030216307A1,U.S. Pat. No. 6,667,060, WO 01/49249, WO 01/49311, WO 01/49249, WO 01/49311, and U.S. Pat. No. 5,877,224.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g. WO 97/11682) via a liposomal formulation (see, e.g., EP 736299,WO 99/59550 and WO 97/13500), via formulations described in WO 03/094886 or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115)). The agents can be administered using high-velocity transdermal particle injection techniques using the hydrogel particle formulation described in U.S. 20020061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal administration. The agents can be administered in the form a suppository or by other vaginal or rectal means. The agents can be administered in a transmembrane formulation as described in WO 90/07923. The agents can be administered non-invasively via the dehydrated particles described in U.S. Pat. No. 6,485,706. The agent can be administered in an enteric-coated drug formulation as described in WO 02/49621. The agents can be administered intranasaly using the formulation described in U.S. Pat. No. 5,179,079. Formulations suitable for parenteral injection are described in WO 00/62759. The agents can be administered using the casein formulation described in U.S. 20030206939 and WO 00/06108. The agents can be administered using the particulate formulations described in U.S. 20020034536.

The agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications. Aerosol formulations are stable dispersions or suspensions of solid material and liquid droplets in a gaseous medium and can be placed into pressurized acceptable propellants, such as hydrofluroalkanes (HFAs, i.e. HFA-134a and HFA-227, or a mixture thereof), dichlorodifluoromethane (or other chlorofluocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion. Pulmonary formulations may also include surfactants which include but are not limited to bile salts and those described in U.S. Pat. No. 6,524,557 and references therein. The surfactants described in U.S. Pat. No. 6,524,557, e.g., a C8-C16 fatty acid salt, a bile salt, a phospholipid, or alkyl saccharide are advantageous in that some of them also reportedly enhance absorption of the compound in the formulation. Also suitable in the invention are dry powder formulations comprising a therapeutically effective amount of active compound blended with an appropriate carrier and adapted for use in connection with a dry-powder inhaler. Absorption enhancers which can be added to dry powder formulations of the present invention include those described in U.S. Pat. No. 6,632,456. WO 02/080884 describes new methods for the surface modification of powders. Aerosol formulations may include U.S. Pat. No. 5,230,884, U.S. Pat. No. 5,292,499, WO 017/8694, WO 01/78696, U.S. 2003019437, U. S. 20030165436, and WO 96/40089 (which includes vegetable oil). Sustained release formulations suitable for inhalation are described in U.S. 20010036481A1, 20030232019A1, and U.S. 20040018243A1 as well as in WO 01/13891, WO 02/067902, WO 03/072080, and WO 03/079885. Pulmonary formulations containing microparticles are described in WO 03/015750, U.S. 20030008013, and WO 00/00176. Pulmonary formulations containing stable glassy state powder are described in U.S. 20020141945 and U.S. Pat. No. 6,309,671. Other aerosol formulations are described in EP 1338272A1 WO 90/09781, U.S. Pat. No. 5,348,730, U.S. Pat. No. 6,436,367, WO 91/04011, and U.S. Pat. No. 6,294,153 and U.S. Pat. No. 6,290,987 describes a liposomal based formulation that can be administered via aerosol or other means. Powder formulations for inhalation are described in U.S. 20030053960 and WO 01/60341. The agents can be administered intranasally as described in U.S. 20010038824.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The agent can be fused to immunoglobulins or albumin, or incorporated into a liposome to improve half-life. The agent can also be conjugated to polyethylene glycol (PEG) chains. Methods for pegylation and additional formulations containing PEG-conjugates (i.e. PEG-based hydrogels, PEG modified liposomes) can be found in Harris and Chess, Nature Reviews Drug Discovery 2: 214-221 and the references therein. The agent can be administered via a nanocochleate or cochleate delivery vehicle (BioDelivery Sciences International). The agents can be delivered transmucosally (i.e. across a mucosal surface such as the vagina, eye or nose) using formulations such as that described in U.S. Pat. No. 5,204,108. The agents can be formulated in microcapsules as described in WO 88/01165. The agent can be administered intra-orally using the formulations described in U.S. 20020055496, WO 00/47203, and U.S. Pat. No. 6,495,120. The agent can be delivered using nanoemulsion formulations described in WO 01/91728A2.

The agents can be a free acid or base, or a pharmacologically acceptable salt thereof. Solids can be dissolved or dispersed inmmediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences, 18th Ed., Mack Publishing Company, 1995.

Methods to increase chemical and/or physical stability of the agents the described herein are found in WO 00/04880, and WO 97/04796 and the references cited therein.

Methods to increase bioavailability of the agents described herein are found in U.S. 20030198619, WO 01/49268, WO 00/32172, and WO 02/064166. Glycyrrhizinate can also be used as an absorption enhancer (see, e.g., EP397447). WO 03/004062 discusses Ulex europaeus I (UEAI) and UEAI mimetics which may be used to target the agents of the invention to the GI tract. The agents described herein and combination therapy agents can be packaged as a kit that includes single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation.

A number of embodiments of the invention have been described. Nevertheless, it will be understood that various modifications may be made without departing from the spirit and scope of the invention.

1. A compound having the formula: 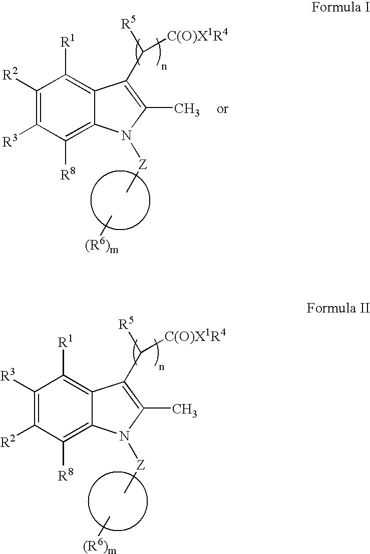
wherein:
R¹ is: H or a halogen;
R² is: H, a halogen, or R^{2B}O— wherein
R^{2B} is selected from:
(a) H;
(b) C₁ to C₆ alkyl or a C₂ to C₆ alkenyl that is optionally independently substituted with one or more halogen; —OH, —NH₂, —C(O)OH; 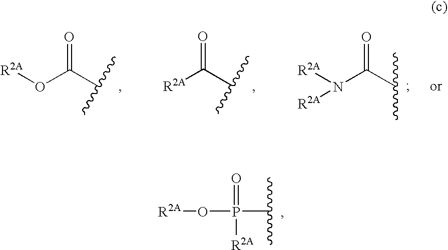
wherein each R^{2A} is independently: H, a C₁ to C₆ alkyl, a C₂ to C₆ alkenyl, a C₂ to C₆ alkynyl, a C₆ to C₁₀ aryl, a C₃ to C₁₀ cycloalkyl, or a C₇ to C₂₀ arylalkyl optionally independently substituted with one or more halogen, —OH, —C(O)OH, or —NH₂;
R³is H or a halogen;
X¹ is —O—, —S—, —N(H)— or —N(H)S(O₂)—;
Z is 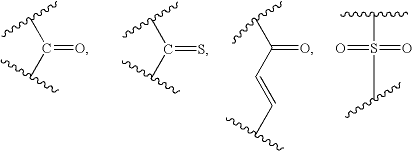
or C;
R⁴ is H; a C₁ to C₁₀ alkyl; a C₂-C₁₀ alkenyl; a C₂-C₁₀ alkynyl; a C₃ to C₈ cycloalkyl; a C₁ to C₆ hydroxyalkyl; a hydroxyl substituted C₆ to C₈ aryl; a primary, secondary or tertiary C₁ to C₆ alkylamino; primary, secondary or tertiary C₆ to C₈ arylamino; C₂ to C₆ alkylcarboxylic acid; a C₁ to C₆ alkylester; a C₆ to C₈ aryl; a C₆ to C₈ arylcarboxylic acid; a C₆ to C₈ arylester; a C₆ to C₈ aryl substituted C, to C₆ alkyl; a 4 to 8 membered heterocyclic alkyl or heteroaryl wherein the heteroatoms are selected from O, S, S(O)₂, N, and S(O); an alkyl-substituted or aryl-substituted a 4 to 8 membered heterocyclic alkyl or heteroaryl wherein the heteroatoms are selected from O, S, S(O)₂, N, and S(O), wherein one or more H within R⁴ can be substituted by a halogen, —OH, or —C(O)OH, —NH₂;
n is 1, 2, 3, 4 or 5;
Each R⁵ is independently: H, an optionally substituted C₁-C₄ alkyl, wherein the substituents are independently selected from a halogen and —OH; 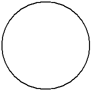
represents a C₃-C₆ saturated carbocycle, a C₆ aryl, C₃-C₆ non-saturated, non-aromatic carbocycle, a 6-membered heteroaryl having 1, 2, 3, 4 or 5 heteroatoms independently selected from O, S, S(O)₂, N, S(O) and N(R⁷) or a 3- to 7-membered saturated or non-saturated heterocycle having 1, 2, 3, 4 or 5 heteroatoms independently selected from O, S, S(O)₂, N, S(O) and N(R⁷);
each R⁶ is independently H, a halogen, —CH₃, —CN, —OCH₃, —SCH₃, —SCF₃, —OCH₂CF₃ or —CH₂CH₃ wherein one or more H can be replaced by a halogen;
m=1, 2, 3, 4, or 5;
R⁷ is: H, a halogen, —CH₃, —CN, —OCH₃, —SCH₃, or —CH₂CH₃ wherein one or more H can be replaced by a halogen; and
R⁸ is: H, a halogen or —CH₃, wherein one or more H can be replaced by a halogen.

2. The compound of claim 1 wherein 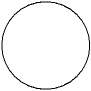
represents a C₃-C₆ saturated carbocycle.

3. The compound of claim 2 wherein the C₃-C₆ saturated carbocycle is selected from cyclohexyl, cyclopentyl, cyclobutyl and cyclopropyl.

4. The compound of claim 1 wherein 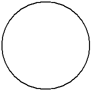
represents a C₃-C₆ non-saturated, non-aromatic carbocycle.

5. The compound of claim 4 wherein the C₃-C₆ non-saturated, non-aromatic carbocycle is selected from a cyclohexenyl, a cyclopentenyl, a cyclobutenyl.

6. The compound of claim 1 wherein 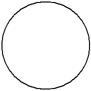
represents a 6-membered heteroaryl.

7. The compound of claim 6 wherein the 6-membered heteroaryl is selected from pyrazine, pyridazine, triazine, tetrazine, and pentazine.

8. The compound of claim 1 wherein 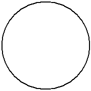
represents a 3- to 7-membered saturated heterocycle.

9. The compound of claim 8 wherein the 6-membered saturated heterocycle is selected from piperidine, piperazine, morpholine, thiomorpholine, thiomorpholine sulfoxide, thiomorpholine sulfone, tetrahydropyran, tetrahydrothiopyran, and dioxane.

10. The compound of claim 1 wherein 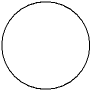
represents a 3- to 7-membered non-saturated heterocycle.

11. The compound of claim 10 wherein the 3- to 7-membered non-saturated heterocycle is selected from: thiphene, furan, pyrrole, thaizole, oxazole, imidizole, isothazole, isoxazole pyrazole, triazole, tetrazole, oxadiazole, oxatriazole and thiadiazole.

12. The compound of claim 1 wherein R¹ is H.

13. The compound of claim 1 wherein R¹ is a halogen.

14. The compound of claim 13 wherein R¹ is F or Cl.

15. The compound of claim 1 wherein R^{2B} is a substituted C₁ to C₆ alkyl or a substituted C₂ to C₆ alkenyl.

16. The compound of claim 1 wherein R^{2B} is not substituted.

17. The compound of claim 1 wherein R^{2B} is a C₁ to C₆ alkyl or a C₂ to C₆ alkenyl optionally substituted with one or more halogen.

18. The compound of claim 1 wherein R^{2B} is a C₁ to C₃ alkyl or alkenyl.

19. The compound of claim 18 wherein R^{2B} is a C₁ to C₃ alkyl.

20. The compound of claim 19 wherein R^{2B} is a methyl group or an ethyl group.

21. The compound of claim 1 wherein R^{2B} is substituted only with a halogen.

22. The compound of claim 1 wherein R² is H.

23. The compound of claim 1 wherein R³is a halogen.

24. The compound of claim 23 wherein R³ is Cl.

25. The compound of claim 23 wherein R³is F.

26. The compound of claim 1 wherein X¹ is —O—.

27. The compound of claim 1 wherein X¹ is —S—.

28. The compound of claim 1 wherein X¹ is —N(H)—

29. The compound of claim 1 wherein X¹ is —N(H)S(O)₂—.

30. The compound of claim 1 wherein Z is

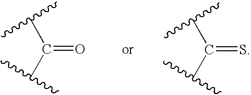

31. The compound of claim 1 wherein Z is 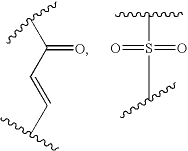
or C.

32. The compound of claim 1 wherein R⁶ is selected from: —CH₃, —CF₂H, —CH₂F, —CF₃, —CN, —OCF₂H, —OCH₃, —SCF₃, —SCF₂H, —SCH₃, —CH₂CH₃ and —OCF₃.

33. The compound of claim 1 wherein R⁷ is selected from: —CH₃, —CF₂H, —CH₂F, —CF₃, —CN, —OCF₂H, —OCH₃, —SCF₃, —SCF₂H, —SCH₃, —CH₂CH₃ and —OCF₃.

34. The compound of claim 1 wherein n is 1 or 2.

35. The compound of claim 1 wherein m is 1 or 2.

36. The compound of claim 1 wherein R⁵ is H.

37. The compound of claim 1 wherein R⁵ is a methyl group or an ethyl group.

38. The compound of claim 1 wherein R⁵ is an unsubstituted methyl group or an unsubstituted ethyl group.

39. The compound of claim 1 wherein R⁴ is H.

40. The compound of claim 1 wherein X¹ is O and R⁴ is H.

41. The compound of claim 1 wherein X¹ is O and R⁴ is other than H.

42. The compound of claim 1 wherein R⁴ is an optionally independently substituted C₃ to C₁₀ branched alkyl.

43. The compound of claim 1 wherein R⁴ is a C₁ to C₁₀ alkyl.

44. The compound of claim 43 wherein R⁴ is a C₄ to C₈ cycloalkyl.

45. The compound of claim 1 wherein R⁴is a C₁ to C₆ hydroxy substituted alkyl.

46. The compound of claim 45 wherein R⁴ is a hydroxyl substituted C₄ to C₈ aryl.

47. The compound of claim 1 wherein R⁴is a primary, secondary or tertiary C₁ to C₆ alkylamino.

48. The compound of claim 1 wherein R⁴ is a primary, secondary or tertiary C₄ to C₈ arylamino.

49. The compound of claim 1 wherein R⁴ is a C₂ to C₆ alkylcarboxylic acid.

50. The compound of claim 1 wherein R⁴ is a C₁ to C₆ alkylester.

51. The compound of claim 50 wherein R⁴ is a branched C₁ to C₆ alkylester.

52. The compound of claim 1 wherein R⁴ is a C₄ to C₈ aryl.

53. The compound of claim 1 wherein R⁴ is a C₄ to C₈ arylcarboxylic acid.

54. The compound of claim 1 wherein R⁴ is a C₄ to C₈ arylester.

55. The compound claim 1 wherein R⁴ is C₄ to C₈ aryl substituted C₁ to C₆ alkyl.

56. The compound of claim 1 wherein R⁴ is a C₄ to C8 heterocyclic alkyl or aryl.

57. The compound of claim 1 wherein R⁴ is an alkyl-substituted or aryl-substituted C₄ to C₈ heterocyclic alkyl or aryl.

58. The compound of claim 1 wherein R⁴ is substituted.

59. The compound of claim 1 wherein R⁴ is unsubstituted.

60. A pharmaceutical composition comprising the compound of claim 1 and a pharmaceutically acceptable carrier.

61. A method for treating inflammation comprising administering a composition comprising the compound of claim 1.

62. A method for treating anxiety comprising administering the compound of claim 1

63. A method for treating a sleep disorder comprising administering the compound of claim 1.

64. A method for treating a respiratory disorder comprising administering the compound of claim 1.

65. The method of claim 65 wherein the respiratory disorder is asthma.

66. A method for inhibiting COX-2 activity in a patient, the method comprising administering the compound of claim 1.

66. A method for inhibiting FAAH activity in a patient, the method comprising administering the compound of claim 1.

67. The method of claim 65 wherein X¹ is O and R⁴ is H.

68. The method of claim 66 wherein X¹ is O and R⁴ is other than H.

69. A method for modulating CRTH2 activity on a patient, the method comprising administering the compound of claim 1.

70. The pharmaceutical composition of claim 60 further comprising an analgesic agent.

71. The pharmaceutical composition of claim 60 further comprising an anti-inflammatory agent.

72. The compound of claim 1 having Formula I.

73. The compound of claim 1 having Formula II.

74. The compound of claim 1 wherein R⁸ is H.

75. The compound of claim 1 having Formula I wherein R⁸ is H.

76. The compound of claim 1 having Formula I wherein Z is

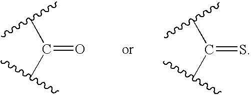

77. The compound of claim 1 having Formula II wherein Z is [chemical structure]
